SEQUENCE LISTING (11 sequences)

SEQ ID NO: 1 - RNA, 21 nt, synthetic construct
  source 1..21: mol_type = other RNA
gtatgacaacagcctcaagtt

SEQ ID NO: 2 - DNA, 5256 bp, synthetic construct
  source 1..5256: mol_type = other DNA
ggtaccgagctcttacgcgtgctagcccgggctcgagatctgcgatctgcatctcaattagtcagcaaccatagtcccgcccctaactccgcccatcccgcccctaactccgcccagttccgcccattctccgccccatcgctgactaattttttttatttatgcagaggccgaggccgcctcggcctctgagctattccagaagtagtgaggaggcttttttggaggcctaggcttttgcaaaaagcttggcattccggtactgttggtaaagccaccatggaagacgccaaaaacataaagaaaggcccggcgccattctatccgctggaagatggaaccgctggagagcaactgcataaggctatgaagagatacgccctggttcctggaacaattgcttttacagatgcacatatcgaggtggacatcacttacgctgagtacttcgaaatgtccgttcggttggcagaagctatgaaacgatatgggctgaatacaaatcacagaatcgtcgtatgcagtgaaaactctcttcaattctttatgccggtgttgggcgcgttatttatcggagttgcagttgcgcccgcgaacgacatttataatgaacgtgaattgctcaacagtatgggcatttcgcagcctaccgtggtgttcgtttccaaaaaggggttgcaaaaaattttgaacgtgcaaaaaaagctcccaatcatccaaaaaattattatcatggattctaaaacggattaccagggatttcagtcgatgtacacgttcgtcacatctcatctacctcccggttttaatgaatacgattttgtgccagagtccttcgatagggacaagacaattgcactgatcatgaactcctctggatctactggtctgcctaaaggtgtcgctctgcctcatagaactgcctgcgtgagattctcgcatgccagagatcctatttttggcaatcaaatcattccggatactgcgattttaagtgttgttccattccatcacggttttggaatgtttactacactcggatatttgatatgtggatttcgagtcgtcttaatgtatagatttgaagaagagctgtttctgaggagccttcaggattacaagattcaaagtgcgctgctggtgccaaccctattctccttcttcgccaaaagcactctgattgacaaatacgatttatctaatttacacgaaattgcttctggtggcgctcccctctctaaggaagtcggggaagcggttgccaagaggttccatctgccaggtatcaggcaaggatatgggctcactgagactacatcagctattctgattacacccgagggggatgataaaccgggcgcggtcggtaaagttgttccattttttgaagcgaaggttgtggatctggataccgggaaaacgctgggcgttaatcaaagaggcgaactgtgtgtgagaggtcctatgattatgtccggttatgtaaacaatccggaagcgaccaacgccttgattgacaaggatggatggctacattctggagacatagcttactgggacgaagacgaacacttcttcatcgttgaccgcctgaagtctctgattaagtacaaaggctatcaggtggctcccgctgaattggaatccatcttgctccaacaccccaacatcttcgacgcaggtgtcgcaggtcttcccgacgatgacgccggtgaacttcccgccgccgttgttgttttggagcacggaaagacgatgacggaaaaagagatcgtggattacgtcgccagtcaagtaacaaccgcgaaaaagttgcgcggaggagttgtgtttgtggacgaagtaccgaaaggtcttaccggaaaactcgacgcaagaaaaatcagagagatcctcataaaggccaagaagggcggaaagatcgccgtgtaattctagagtcggggcggccggccgcttcgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtaaaatcgataaggatctgaacgatggagcggagaatgggcggaactgggcggagttaggggcgggatgggcggagttaggggcgggactatggttgctgactaattgagatgcatgctttgcatacttctgcctgctggggagcctggggactttccacacctggttgctgactaattgagatgcatgctttgcatacttctgcctgctggggagcctggggactttccacaccctaactgacacacattccacagcggatccgtcgaccgatgcccttgagagccttcaacccagtcagctccttccggtgggcgcggggcatgactatcgtcgccgcacttatgactgtcttctttatcatgcaactcgtaggacaggtgccggcagcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttcccattcgccattcaggctgcgcaactgttgggaagggcgatcggtgcgggcctcttcgctattacgccagcccaagctaccatgataagtaagtaatattaaggtacgggaggtacttggagcggccgcaataaaatatctttattttcattacatctgtgtgttggttttttgtgtgaatcgatagtactaacatacgctctccatcaaaacaaaacgaaacaaaacaaactagcaaaataggctgtccccagtgcaagtgcaggtgccagaacatttctctatcgata

SEQ ID NO: 3 - RNA, 21 nt, synthetic construct
  source 1..21: mol_type = other RNA
cttgaggctgttgtcatactt

SEQ ID NO: 4 - DNA, 20 bp, synthetic construct
  source 1..20: mol_type = other DNA
cttcttttgcgtcgccagcc

SEQ ID NO: 5 - DNA, 20 bp, synthetic construct
  source 1..20: mol_type = other DNA
gttctcagccttgacggtgc

SEQ ID NO: 6 - DNA, 18 bp, synthetic construct
  source 1..18: mol_type = other DNA
cctggcacccagcacaat

SEQ ID NO: 7 - DNA, 18 bp, synthetic construct
  source 1..18: mol_type = other DNA
gggccggactcgtcatac

SEQ ID NO: 8 - RNA, 25 nt, synthetic construct
  source 1..25: mol_type = other RNA
gaatgagcagctgatctgtgcaaat

SEQ ID NO: 9 - RNA, 25 nt, synthetic construct
  source 1..25: mol_type = other RNA
atttgcacagatcagctgctcattc

SEQ ID NO: 10 - RNA, 21 nt, synthetic construct
  source 1..21: mol_type = other RNA
  modified_base 2: mod_base = OTHER; note = 2'-O-methyl modification
  modified_base 4: mod_base = OTHER; note = 2'-O-methyl modification
  modified_base 6: mod_base = um
  modified_base 8: mod_base = um
  modified_base 10: mod_base = cm
  modified_base 12: mod_base = um
  modified_base 14: mod_base = cm
  modified_base 16: mod_base = cm
  modified_base 18: mod_base = OTHER; note = 2'-O-methyl modification
  modified_base 20: mod_base = OTHER; note = 2'-O-methyl modification
  modified_base 1: mod_base = OTHER; note = 2'-Deoxy-2'-fluoro modification
  modified_base 3: mod_base = OTHER; note = 2'-Deoxy-2'-fluoro modification
  modified_base 5: mod_base = OTHER; note = 2'-Deoxy-2'-fluoro modification
  modified_base 7: mod_base = OTHER; note = 2'-Deoxy-2'-fluoro modification
  modified_base 9: mod_base = OTHER; note = 2'-Deoxy-2'-fluoro modification
  modified_base 11: mod_base = OTHER; note = 2'-Deoxy-2'-fluoro modification
  modified_base 13: mod_base = OTHER; note = 2'-Deoxy-2'-fluoro modification
  modified_base 15: mod_base = OTHER; note = 2'-Deoxy-2'-fluoro modification
  modified_base 17: mod_base = OTHER; note = 2'-Deoxy-2'-fluoro modification
  modified_base 19: mod_base = OTHER; note = 2'-Deoxy-2'-fluoro modification
  modified_base 21: mod_base = OTHER; note = 2'-Deoxy-2'-fluoro modification
aacagtgttcttgctctataa

SEQ ID NO: 11 - RNA, 23 nt, synthetic construct
  source 1..23: mol_type = other RNA
  modified_base 1: mod_base = um
  modified_base 3: mod_base = OTHER; note = 2'-O-methyl modification
  modified_base 5: mod_base = OTHER; note = 2'-O-methyl modification
  modified_base 7: mod_base = OTHER; note = 2'-O-methyl modification
  modified_base 9: mod_base = cm
  modified_base 11: mod_base = OTHER; note = 2'-O-methyl modification
  modified_base 13: mod_base = OTHER; note = 2'-O-methyl modification
  modified_base 15: mod_base = cm
  modified_base 17: mod_base = cm
  modified_base 19: mod_base = gm
  modified_base 21: mod_base = um
  modified_base 23: mod_base = um
  modified_base 2: mod_base = OTHER; note = 2'-Deoxy-2'-fluoro modification
  modified_base 4: mod_base = OTHER; note = 2'-Deoxy-2'-fluoro modification
  modified_base 6: mod_base = OTHER; note = 2'-Deoxy-2'-fluoro modification
  modified_base 8: mod_base = OTHER; note = 2'-Deoxy-2'-fluoro modification
  modified_base 10: mod_base = OTHER; note = 2'-Deoxy-2'-fluoro modification
  modified_base 12: mod_base = OTHER; note = 2'-Deoxy-2'-fluoro modification
  modified_base 14: mod_base = OTHER; note = 2'-Deoxy-2'-fluoro modification
  modified_base 16: mod_base = OTHER; note = 2'-Deoxy-2'-fluoro modification
  modified_base 18: mod_base = OTHER; note = 2'-Deoxy-2'-fluoro modification
  modified_base 20: mod_base = OTHER; note = 2'-Deoxy-2'-fluoro modification
  modified_base 22: mod_base = OTHER; note = 2'-Deoxy-2'-fluoro modification
  modified_base 21^22: mod_base = OTHER; note = thiophosphate bond
  modified_base 22^23: mod_base = OTHER; note = thiophosphate bond
ttatagagcaagaacactgtttt

(19) United States
(12) Patent Application Publication
(10) Pub. No.: US 2025/0319035 A1
(43) Pub. Date: Oct. 16, 2025

(54) CATIONIC LIPID NANOPARTICLE HAVING HIGH TRANSFECTION EFFICIENCY AND PREPARATION METHOD THEREFOR

(71) Applicants: Shuwen TONG, Changping District, Beijing (CN); BEIJING D-NANO PHARMA CO., LTD, Changping District, Beijing (CN)

(72) Inventor: Shuwen TONG, Beijing (CN)

(73) Assignees: Shuwen TONG, Changping District, Beijing (CN); BEIJING D-NANO PHARMA CO., LTD, Changping District, Beijing (CN)

(21) Appl. No.: 18/861,755

(22) PCT Filed: Apr. 26, 2023
(86) PCT No.: PCT/CN2023/090998

§ 371 Date: Oct. 30, 2024

(30) Foreign Application Priority Data
Apr. 30, 2022 (WO) PCT/CN2022/090833

(51) Int. Cl. A61K 9/51; A61K 31/713

(52) U.S. Cl. CPC A61K 9/5115; A61K 9/5123; A61K 9/5146; A61K 31/713

(57) ABSTRACT

A nucleic acid-loaded calcium-containing cationic lipid nanoparticle, comprising a cationic lipid, a neutral lipid, a PEGylated lipid, and cholesterol and/or a cholesterol ester. The cationic lipid nanoparticle can be used for preparing a gene-based drug for local injection into the body or a nucleic acid vaccine for local or systemic injection into the body.

### TECHNICAL FIELD

The present invention relates to a calcium-containing cationic lipid nanoparticle and a preparation method therefor, in particular to a calcium-containing cationic lipid nanoparticle containing calcium ions in an inner core and used for loading a nucleic acid. The calcium-containing cationic lipid nanoparticle for loading a nucleic acid as disclosed by the present invention has the properties of high transfection efficiency and specific targeting to a liver organ.

### BACKGROUND ART

Gene therapy refers to introducing foreign genes (DNAs or RNAs) into target cells to correct or compensate for diseases caused by defect and abnormal genes in order to achieve therapeutic purposes. Gene therapy can be divided into in vivo therapy and in vitro therapy. In vivo therapy refers to direct application of a gene in vivo for gene delivery to target cells in vivo, and in vitro therapy refers to a method in which a modified gene is transferred to cells in vitro to endow the cells with new characteristics, and the modified cells are then introduced into the body. Unlike chemical drugs and protein drugs, gene therapy has made significant progress in the past several decades in the treatment of genetic diseases that can not be treated before, including tumor cell therapy, gene therapy for genetic diseases, prevention and treatment of infectious diseases, and other areas of treating critical diseases, due to clear and controllable targets and long-term effectiveness upon single administration.

However, due to ubiquitous nucleases throughout the body, the stability of the gene itself is extremely poor in vivo, and since the gene is a negatively charged macromolecule, it is challenging for the gene to enter a cell and escape from an endosome, thus limiting the wide application of gene therapy drugs. It is critically effective to develop an appropriate gene delivery means for the successful application of gene therapies. At present, gene delivery mainly includes physical methods, chemical methods, and viral vector delivery. Among them, physical methods mainly include electrotransfection, gene gun, and other tools, which are suitable for transfection in vitro or gene delivery in a small amount at specific sites in vivo, and the large-scale application thereof is limited. Viral delivery vectors are one of the most commonly used gene delivery tools in vivo and in vitro. Several viral vector gene therapy drugs have been launched on the market or applied in clinical research and include adeno-associated viruses, lentiviruses, etc. However, extensive application of viral vectors to gene therapy is challenging due to the fact that the delivery of genes by viral vectors has disadvantages, such as the risk of causing cancer by random insertion into genomes in vivo, limited size of the gene to be delivered (adeno-associated virus is generally less than 5 kb, and lentivirus is 8 kb), immunogenicity, high cost of quality control, etc. Chemical delivery methods for gene therapy are mainly realized by using non-viral vectors or chemical modification of nucleic acids. Generally, by subjecting siRNAs to GalNac-ESC modification, liver-targeted delivery of siRNAs can be achieved. However, it is difficult to achieve targeted and stable delivery of long-chain mRNAs, DNAs, etc., simply by chemical modification, so developing non-viral vectors for gene therapy is currently in a key research field.

Non-viral vectors for gene delivery mainly include lipid nanovectors, polymer vectors, polypeptide delivery vectors, and inorganic nanoparticle vectors. Since the last few vectors have immunogenicity, poor delivery efficiency, toxicity, etc, no gene therapy products in which such vectors are successfully applied are available on the market up to now. Non-viral vectors used in gene therapy products that are currently available on the market all involve lipid nanoparticle (LNP) technology and include an siRNA drug (brand name Onpattro) approved by the FDA in 2018 for the treatment of polyneuropathy and COVID-19 mRNA vaccines (brand names COMIRNATY and Spikevax) that have been marketed over the past two years. The gene delivery efficiency and safety thereof have been fully validated in clinical trials. LNPs available on the market are composed of four components: an ionizable cationic lipid, a neutral phospholipid, cholesterol, and a PEGylated lipid. The ionizable cationic lipid is used for interaction with a negatively charged gene under acidic conditions to achieve a high gene encapsulation effect. In a neutral environment, it mainly exists in a non-ionized form in an inner core of the LNP, making the LNP present a near neutral surface to avoid positive charge-mediated toxicity and rapid clearance. In addition, it interacts with the membrane of an endosome to mediate endosomal escape during endosomal acidification. The neutral lipid and cholesterol mainly exist in the outer layer of the LNP, and the PEGylated lipid avoids LNP aggregation. However, the current research shows that after LNPs have entered cells, the proportion of genes that achieve endosomal escape is less than 5% relative to the total genes, which leads to a very low gene transfection efficiency, hindering the application of LNPs as a gene therapy vector.

Ca²⁺ is reported to have the function of destabilizing the membrane of endosomes, so adding a large amount of Ca²⁺ to a cell culture environment is reported to increase the gene transfection efficiency of lipid nanoparticles. Calcium phosphate precipitation method proposed in 1973 as a classic method for gene transfection in vitro is still currently one of the most commonly used methods for gene transfection in vitro. However, the calcium phosphate precipitation method cannot control the size and degree of precipitation, the formed precipitate easily aggregates, the transfection effect is greatly influenced by experimental conditions, the reproducibility is poor, and it is impossible to apply same to gene delivery in vivo. On this basis, developing various calcium-containing nanoparticles is an important direction of the development of inorganic nanoparticles. By modifying the surface of the formed coprecipitate of calcium phosphate or calcium carbonate and gene with a polymer or lipid, the precipitate can be stabilized and aggregation can be avoided; however, such vectors still have significant problems in stability and safety. In addition, there are also studies in which negatively charged phospholipids, such as phosphatidylserine, phosphatidylglycerol or phosphatidic acid, instead of phosphate radicals are used to form precipitates with calcium; however, such calcium precipitates also have the problem of difficult control of particle size and stability, and stable gene transfection cannot be achieved.

In the present study, lipid nanovectors are used to encapsulate Ca²⁺ and gene, by which stable and efficient encapsulation can be achieved, the particle size is controllable, the transfection efficiency in vivo and in vitro is significantly higher than the effect of LNPs on gene delivery, and there is no obvious toxic effect.

### SUMMARY OF THE INVENTION

The present invention relates to a calcium-containing cationic lipid nanoparticle and a preparation method therefor, in particular to a calcium-containing cationic lipid nanoparticle containing calcium ions in an inner core and used for loading a nucleic acid. The calcium-containing cationic lipid nanoparticle for loading a nucleic acid as disclosed by the present invention has the properties of high transfection efficiency and specific targeting to a liver organ. The present invention is implemented by the technical solutions of the following aspects.

Aspect 1. A nucleic acid-loaded calcium-containing cationic lipid nanoparticle, wherein the inner core of the cationic lipid nanoparticle contains calcium ions in a non-precipitated state.

Aspect 2. The calcium-containing cationic lipid nanoparticle according to aspect 1, wherein the concentration of calcium in the whole formulation is 0.1-150 mmol/L; preferably is 1-10 mmol/L, 10-100 mmol/L, or 100-150 mmol/L; more preferably is 1-10 mmol/L, 10-30 mmol/L, 30-50 mmol/L, 50-70 mmol/L, 70-90 mmol/L, 90-110 mmol/L, 110-130 mmol/L, or 130-150 mmol/L.

Aspect 3. The calcium-containing cationic lipid nanoparticle according to aspect 1, wherein the local concentration of calcium in the inner core is 50-800 mmol/L;
preferably is 50-100 mmol/L, 100-200 mmol/L, 200-400 mmol/L, or 400-800 mmol/L;
more preferably is 50-100 mmol/L, 100-150 mmol/L, 150-200 mmol/L, 200-250 mmol/L, 250-300 mmol/L, 350-400 mmol/L, 400-450 mmol/L, 450-500 mmol/L, 500-550 mmol/L, 550-600 mmol/L, 600-650 mmol/L, 650-700 mmol/L, 700-750 mmol/L, or 750-800 mmol/L.

Aspect 4. The calcium-containing cationic lipid nanoparticle according to aspect 1 or 2, wherein the molar ratio of the calcium ions in the non-precipitated state to the lipids in the inner core of the lipid particle is 1:(0.01-20), preferably is 1:(0.1-10), preferably is 1:(1-10), or preferably is 1:(0.1-1).

Aspect 5. The calcium-containing cationic lipid nanoparticle according to any one of the preceding aspects, wherein the calcium ions are derived from calcium salts, preferably are derived soluble calcium salts, more preferably are derived calcium acetate, calcium chloride, calcium sodium EDTA, calcium gluconate, calcium dihydrogen phosphate, calcium nitrate, calcium bicarbonate, calcium bisulfate, calcium bisulfite, calcium bromide, calcium iodide, calcium citrate, calcium lactate, and calcium gluconate, further more preferably are derived calcium acetate, calcium sodium EDTA, calcium gluconate, calcium citrate, calcium lactate, and calcium gluconate, further more preferably are derived calcium acetate.

Aspect 6. The calcium-containing cationic lipid nanoparticle according to any one of the preceding aspects, wherein the calcium ions are derived from a calcium salt solution with a concentration of 50-1000 mmol/L; preferably, the calcium ions are derived from a calcium salt solution with a concentration of 50-150 mmol/L, a calcium salt solution with a concentration of 150-300 mmol/L, a calcium salt solution with a concentration of 300-500 mmol/L, or a calcium salt solution with a concentration of 500-800 mmol/L; more preferably, the calcium ions are derived from a calcium salt solution with a concentration of 100±50 mmol/L, a calcium salt solution with a concentration of 200±50 mmol/L, a calcium salt solution with a concentration of 300±50 mmol/L, a calcium salt solution with a concentration of 400±50 mmol/L, a calcium salt solution with a concentration of 500±50 mmol/L, a calcium salt solution with a concentration of 600±50 mmol/L, a calcium salt solution with a concentration of 700±50 mmol/L, a calcium salt solution with a concentration of 800±50 mmol/L, or a calcium salt solution with a concentration of 900-50 mmol/L.

Aspect 7. The calcium-containing cationic lipid nanoparticle according to any one of the preceding aspects, wherein the calcium ions exist in the form of calcium ions in an inner core aqueous phase solution.

Aspect 8. The calcium-containing cationic lipid nanoparticle according to any one of the preceding aspects, wherein a substance to be delivered by the calcium-containing cationic lipid nanoparticle is nucleic acid, preferably is plasmid DNA, single-stranded DNA, double-stranded DNA, siRNA, shRNA, aiRNA, miRNA, mRNA, circular RNA, tRNA, rRNA, vRNA, gRNA, an aptamer, a ribozyme, an oligonucleotide, or any combination thereof.

Aspect 9. The calcium-containing cationic lipid nanoparticle according to any one of the preceding aspects, wherein the molar number of phosphate radicals in the nucleic acid:the molar number of positive charges in the cationic lipid is 1:(0.5-20), preferably is 1:(1-10), more preferably is 1:(1.5-6), more preferably 1:(1.5-3) or 1:(3-6).

Aspect 10. The calcium-containing cationic lipid nanoparticle according to any one of the preceding aspects, wherein the mass ratio of the nucleic acid to the lipid is 1:(1-100), preferably 1:(5-90), more preferably 1:(10-70), further preferably 1:(10-30).

Aspect 11. The calcium-containing cationic lipid nanoparticle according to any one of the preceding aspects, wherein the length of the substance to be delivered by the calcium-containing cationic lipid nanoparticle is about 15-30000 bases (base pairs); preferably 15-60, 60-120, 120-250, 250-500, 500-1000, 1000-2000, 2000-4000, 4000-8000, 8000-15000, 15000-20000, 20000-25000, or 25000-30000 bases (base pairs); more preferably 15-60, 15-50, 15-40, 15-30, 15-25, 19-25, 20-30, 20-50, 20-80, 30-50, 30-80, 30-120, 50-100, 50-150, 50-250, 100-200, 100-300, 100-500, 200-500, 200-1000, 300-800, 300-1500, 1000-3000, 1000-5000, 1000-8000, 5000-10000, 5000-15000, 5000-20000, 10000-25000, or 10000-30000 bases (base pairs).

Aspect 12. The calcium-containing cationic lipid nanoparticle according to any one of the preceding aspects, wherein the amount of the loaded nucleic acid is 5 μg/ml-10 mg/ml;
preferably, the amount of the loaded nucleic acid is 5-10 μg/ml, 10-20 μg/ml, 20-40 μg/ml, 40-80 μg/ml, 80-150 μg/ml, 150-300 μg/ml, 300-400 μg/ml, 400-800 μg/ml, 800 μg/ml-1 mg/ml, 1-1.5 mg/ml, 1.5-2 mg/ml, 2-4 mg/ml, 4-6 mg/ml, 6-8 mg/ml, or 8-10 mg/ml;
more preferably, the amount of the loaded nucleic acid is 50±50 μg/ml, 100±50 μg/ml, 200±50 μg/ml, 300±50 μg/ml, 400±50 μg/ml, 500±50 μg/ml, 600±50 μg/ml, 700±50 μg/ml, 800±50 μg/ml, 900±50 μg/ml, 1000±50 μg/ml, 1500±50 μg/ml, 2000±50 μg/ml, 2500±50 μg/ml, 3000±50 μg/ml, 4000±50 μg/ml, 5000±50 μg/ml, 6000±50 μg/ml, 7000±50 μg/ml, 8000±50 μg/ml, 9000±50 μg/ml, or 10000±50 μg/ml.

Aspect 13. The calcium-containing cationic lipid nanoparticle according to any one of the preceding aspects, wherein the lipids constituting the cationic lipid nanoparticle comprise one of or a combination of an ionizable cationic lipid, cholesterol and/or cholesterol ester, neutral lipid, and PEGylated lipid;
preferably, the lipids constituting the cationic lipid nanoparticle comprise the following lipids:
(1) cationic lipid selected from ionizable cationic lipids;
(2) cholesterol lipid selected from cholesterol and/or cholesterol esters;
(3) neutral lipid selected from phospholipids, fatty acid glycerides, or glycolipids, or any combination thereof; and
optionally, (4) PEGylated lipid;
more preferably, the lipids constituting the cationic lipid nanoparticle comprise:
(1) cationic lipid selected from ionizable cationic lipids;
(2) cholesterol lipid selected from cholesterol;
(3) neutral lipid selected from phospholipids; and
optionally, (4) PEGylated lipid.

Aspect 14. The calcium-containing cationic lipid nanoparticle according to any one of the preceding aspects, wherein the lipids constituting the cationic lipid nanoparticle include a combination of the following components in % mole:
(1) cationic lipid: 1-90%,
(2) cholesterol lipid: 1-90%,
(3) neutral lipid: 1-90%, and
(4) PEGylated lipid: 0.1-20%.

Aspect 15. The calcium-containing cationic lipid nanoparticle according to any one of the preceding aspects, wherein the cationic lipid nanoparticle is free of non-PEG-group-modified negatively charged lipid.

Aspect 16. The calcium-containing cationic lipid nanoparticle according to any one of the preceding aspects, wherein the cationic lipid is selected from ionizable cationic lipids; preferably, the ionizable cationic lipid is selected from DSDMA, DLinDMA, DLenDMA, DODMA, A6, OF-02, A18-Iso5-2DC18, 98N₁₂-5, 9A1P9, C12-200, cKK-E12, 7C1, G0-C14, L319, 304O₁₃, OF-Deg-Lin, 306-O12B, 306Oᵢ₁₀, FTT5, SM102, ALC-0315, A9, Lipid 2,2(8,8)4CCH3, CL1, LP01, DLin-MC3-DMA, or analogs thereof.

Aspect 17. The calcium-containing cationic lipid nanoparticle according to any one of the preceding aspects, wherein the neutral phospholipid is selected from one or more of egg yolk lecithin, soybean lecithin, hydrogenated soybean lecithin, phosphatidylcholine, phosphatidylethanolamine, phosphatidylinositol, distearoylphosphatidylcholine, dimyristoylphosphatidylcholine, dipalmitoylphosphatidylcholine, dioleoylphosphatidylcholine, distearoylphosphatidylethanolamine, dimyristoylphosphatidylethanolamine, dipalmitoylphosphatidylethanolamine, dioleoylphosphatidylethanolamine, distearoylphosphatidylinositol, dimyristoylphosphatidylinositol, dipalmitoylphosphatidylinositol, dioleoylphosphatidylinositol, 9A1P9, and 10A1P10; preferably one or more of phosphatidylcholine, egg yolk lecithin, soybean lecithin, hydrogenated soybean lecithin, and phosphatidylethanolamine.

Aspect 18. The calcium-containing cationic lipid nanoparticle according to any one of the preceding aspects, wherein the PEGylated lipid is selected from methoxy polyethylene (mPEG-DSPE), methoxy polyethylene glycol-distearoylphosphatidylethanolamine glycol-dioleoylphosphatidylethanolamine (mPEG-DOPE), methoxy polyethylene glycol-dipalmitoylphosphatidylethanolamine (mPEG-DPPE), polyethylene glycol-dilauroylglycerol (PEG-DAG), polyethylene glycol-dimyristoylglycerol (PEG-DMG), polyethylene glycol-dipalmitoylglycerol (PEG-DPG), polyethylene glycol-distearoylglycerol (PEG-DSG), polyethylene glycol-dioleoylglycerol (PEG-DOG), polyethylene glycol-dilinoleylglycerol (PEG-DLinG), polyethylene glycol-dilauroylpropylamine (PEG-DAA), polyethylene glycol-dimyristoylpropylamine (PEG-DMA), polyethylene glycol-dipalmitoylpropylamine (PEG-DPA), polyethylene glycol-dioleoylpropylamine (PEG-DOA), polyethylene glycol-dilinoleylpropylamine (PEG-DLinA), polyethylene glycol-ceramide (PEG-ceramide), stearoyl polyethylene glycol ester, vitamin E polyethylene glycol succinate (TPGS), and any combination thereof;
wherein the PEG is a PEG group with a degree of polymerization selected from 3-100; preferably, the PEG is a PEG group with a degree of polymerization selected from 3-50 or 50-100; more preferably, the PEG is a PEG group with a degree of polymerization selected from 3-10, 10-20, 20-30, 30-40, 40-50, 50-60, 60-70, 70-80, 80-90, or 90-100; more preferably, the PEG is a PEG group with a degree of polymerization selected from about 5, about 10, about 15, about 20, about 25, about 30, about 35, about 40, about 45, about 50, about 55, about 60, about 65, about 70, about 75, about 80, about 85, about 90, about 95, or about 100.

Aspect 19. The calcium-containing cationic lipid nanoparticle according to any one of the preceding aspects, wherein the particle size of the calcium-containing cationic lipid nanoparticle is 25-1000 nm; preferably, the particle size of the lipid nanoparticle is 25-500 nm or 500-1000 nm; more preferably, the particle size of the lipid nanoparticle is 25-75 nm, 75-125 nm, 125-175 nm, 175-225 nm, 225-275 nm, 275-350 nm, 350-500 nm, 500-800 nm, or 800-1000 nm; more preferably, the particle size of the lipid nanoparticle is 40±10 nm, 50±10 nm, 60=10 nm, 70±10 nm, 80±10 nm, 90±10 nm, 100±10 nm, 110±10 nm, 120=10 nm, 125±10 nm, 130±10 nm, 140±10 nm, 150=10 nm, 160±10 nm, 170±10 nm, 180±10 nm, 190±10 nm, 200±10 nm, 210±10 nm, 220±10 nm, or 250=10 nm.

Aspect 20. The calcium-containing cationic lipid nanoparticle according to any one of the preceding aspects, wherein the nucleic acid encapsulation efficiency of the calcium-containing cationic lipid nanoparticle is >30%; preferably >40%; preferably >50%; preferably >60%; preferably >70%; preferably >80%; preferably >90%; more preferably >95%; more preferably >97%; more preferably >98%.

Aspect 21. The calcium-containing cationic lipid nanoparticle according to any one of the preceding aspects, wherein the calcium-containing cationic lipid nanoparticle is selected from the following nanoparticle formulations:
siRNA-loaded calcium-containing cationic lipid nanoparticle, with cationic lipid (preferably DLin-MC3-DMA), cholesterol, neutral phospholipid (preferably DSPC), and PEGylated lipid (preferably PEG2000-DMG) as lipids and calcium acetate as calcium ion source;
mRNA-loaded calcium-containing cationic lipid nanoparticle, with cationic lipid (preferably DLin-MC3-DMA), cholesterol, neutral phospholipid (preferably DSPC), and PEGylated lipid (preferably PEG2000-DMG) as lipids and calcium acetate as calcium ion source; and DNA-loaded calcium-containing cationic lipid nanoparticle, with cationic lipid (preferably DLin-MC3-DMA), cholesterol, neutral phospholipid (preferably DSPC), and PEGylated lipid (preferably PEG2000-DMG) as lipids and calcium acetate as calcium ion source.

Aspect 22. The calcium-containing cationic lipid nanoparticle according to any one of the preceding aspects, wherein the calcium-containing cationic lipid nanoparticle can enhance the transfection efficiency of the loaded nucleic acid.

Aspect 23. The calcium-containing cationic lipid nanoparticle according to any one of the preceding aspects, wherein the calcium-containing cationic lipid nanoparticle targets liver, lung, or spleen.

Aspect 24. A calcium-containing cationic lipid nanoparticle composition, wherein comprises lipid nanoparticles containing non-precipitated state calcium ions in the inner core, nucleic acid-loaded cationic lipid nanoparticles, and/or optionally nucleic acid-loaded cationic lipid nanoparticles containing non-precipitated state calcium ions in the inner core.

Aspect 25. The calcium-containing cationic lipid nanoparticle composition according to aspect 24, wherein the concentration of calcium in the whole formulation is 0.1-150 mmol/L; preferably 1-10 mmol/L, 10-100 mmol/L, or 100-150 mmol/L; more preferably 1-10 mmol/L, 10-30 mmol/L, 30-50 mmol/L, 50-70 mmol/L, 70-90 mmol/L, 90-110 mmol/L, 110-130 mmol/L, or 130-150 mmol/L.

Aspect 26. The calcium-containing cationic lipid nanoparticle composition according to either aspect 24 or 25, wherein the local concentration of calcium in the inner core is 50-800 mmol/L;
preferably 50-100 mmol/L, 100-200 mmol/L, 200-400 mmol/L, or 400-800 mmol/L;
more preferably 50-100 mmol/L, 100-150 mmol/L, 150-200 mmol/L, 200-250 mmol/L, 250-300 mmol/L, 350-400 mmol/L, 400-450 mmol/L, 450-500 mmol/L, 500-550 mmol/L, 550-600 mmol/L, 600-650 mmol/L, 650-700 mmol/L, 700-750 mmol/L, or 750-800 mmol/L.

Aspect 27. The calcium-containing cationic lipid nanoparticle composition according to any one of aspects 24-26, wherein the molar ratio of the calcium ions in the non-precipitated state in the inner core of the lipid particles to all the lipids contained in the composition is 1:(0.01-20), preferably 1:(0.1-10), preferably 1:(1-10), or preferably 1:(0.1-1).

Aspect 28. The calcium-containing cationic lipid nanoparticle composition according to any one of aspects 24-27, wherein the calcium ions are derived from calcium salts, preferably soluble calcium salts, more preferably calcium acetate, calcium chloride, calcium sodium EDTA, calcium gluconate, calcium dihydrogen phosphate, calcium nitrate, calcium bicarbonate, calcium bisulfate, calcium bisulfite, calcium bromide, calcium iodide, calcium citrate, calcium lactate, and calcium gluconate, further more preferably calcium acetate, calcium sodium EDTA, calcium gluconate, calcium citrate, calcium lactate, and calcium gluconate, further more preferably calcium acetate.

Aspect 29. The calcium-containing cationic lipid nanoparticle composition according to any one of aspects 24-28, wherein the calcium ions are derived from a calcium salt solution with a concentration of 50-1000 mmol/L; preferably, the calcium ions are derived from a calcium salt solution with a concentration of 50-150 mmol/L, a calcium salt solution with a concentration of 150-300 mmol/L, a calcium salt solution with a concentration of 300-500 mmol/L, or a calcium salt solution with a concentration of 500-800 mmol/L; more preferably, the calcium ions are derived from a calcium salt solution with a concentration of 100±50 mmol/L, a calcium salt solution with a concentration of 200-50 mmol/L, a calcium salt solution with a concentration of 300±50 mmol/L, a calcium salt solution with a concentration of 400±50 mmol/L, a calcium salt solution with a concentration of 500±50 mmol/L, a calcium salt solution with a concentration of 600±50 mmol/L, a calcium salt solution with a concentration of 700±50 mmol/L, a calcium salt solution with a concentration of 800±50 mmol/L, or a calcium salt solution with a concentration of 900±50 mmol/L.

Aspect 30. The calcium-containing cationic lipid nanoparticle composition according to any one of aspects 24-29, wherein the substance to be delivered by the calcium-containing cationic lipid nanoparticle composition is nucleic acid, preferably plasmid DNA, single-stranded DNA, double-stranded DNA, siRNA, shRNA, aiRNA, miRNA, mRNA, circular RNA, tRNA, rRNA, vRNA, gRNA, aptamer, ribozyme, oligonucleotide, or any combination thereof;
preferably, the molar number of phosphate radicals in the nucleic acid:the molar number of positive charges of all the cationic lipids contained in the composition is 1:(0.5-20), preferably 1:(1-10), more preferably 1:(1.5-6), more preferably 1:(1.5-3), or 1:(3-6);
more preferably, the mass ratio of the nucleic acid to the lipids is 1:(1-100), preferably 1:(5-90), more preferably 1:(10-70), further preferably 1:(10-30).

Aspect 31. The calcium-containing cationic lipid nanoparticle composition according to any one of aspects 24-30, wherein the length of the substance to be delivered by the calcium-containing cationic lipid nanoparticle is about 15-30000 bases (base pairs); preferably 15-60, 60-120, 120-250, 250-500, 500-1000, 1000-2000, 2000-4000, 4000-8000, 8000-15000, 15000-20000, 20000-25000, or 25000-30000 bases (base pairs); more preferably 15-60, 15-50, 15-40, 15-30, 15-25, 19-25, 20-30, 20-50, 20-80, 30-50, 30-80, 30-120, 50-100, 50-150, 50-250, 100-200, 100-300, 100-500, 200-500, 200-1000, 300-800, 300-1500, 1000-3000, 1000-5000, 1000-8000, 5000-10000, 5000-15000, 5000-20000, 10000-25000, or 10000-30000 bases (base pairs);
preferably, the amount of the loaded nucleic acid is 5 μg/ml-10 mg/ml; preferably, the amount of the loaded nucleic acid is 5-10 μg/ml, 10-20 μg/ml, 20-40 μg/ml, 40-80 μg/ml, 80-150 μg/ml, 150-300 μg/ml, 300-400 μg/ml, 400-800 μg/ml, 800 μg/ml-1 mg/ml, 1-1.5 mg/ml, 1.5-2 mg/ml, 2-4 mg/ml, 4-6 mg/ml, 6-8 mg/ml, or 8-10 mg/ml; more preferably, the amount of the loaded nucleic acid is 50±50 μg/ml, 100±50 μg/ml, 200±50 μg/ml, 300±50 μg/ml, 400±50 μg/ml, 500±50 μg/ml, 600±50 μg/ml, 700±50 μg/ml, 800±50 μg/ml, 900±50 μg/ml, 1000±50 μg/ml, 1500±50 μg/ml, 2000±50 μg/ml, 2500±50 μg/ml, 3000±50 μg/ml, 4000±50 μg/ml, 5000±50 μg/ml, 6000±50 μg/ml, 7000±50 μg/ml, 8000±50 μg/ml, 9000±50 μg/ml, or 10000±50 μg/ml.

Aspect 32. The calcium-containing cationic lipid nanoparticle composition according to any one of aspects 24-31, wherein the lipids constituting the nucleic acid-loaded cationic lipid nanoparticles and/or the optional nucleic acid-loaded cationic lipid nanoparticles containing calcium ions in a non-precipitated state in the inner core include one of or a combination of the following components: ionizable cationic lipid, cholesterol and/or cholesterol ester, neutral lipid, and PEGylated lipid;
preferably, the lipids constituting the nucleic acid-loaded cationic lipid nanoparticles and/or the optional nucleic acid-loaded cationic lipid nanoparticles containing calcium ions in a non-precipitated state in the inner core include the following lipids:
(1) cationic lipid selected from ionizable cationic lipids;
(2) cholesterol lipid selected from cholesterol and/or cholesterol esters;
(3) neutral lipid selected from phospholipids, fatty acid glycerides, or glycolipids, or any combination thereof; and
optionally, (4) PEGylated lipid;
more preferably, the lipids constituting the nucleic acid-loaded cationic lipid nanoparticles and/or optionally the nucleic acid-loaded cationic lipid nanoparticles containing calcium ions in a non-precipitated state in the inner core include:
(1) cationic lipid selected from ionizable cationic lipids;
(2) cholesterol lipid selected from cholesterol;
(3) neutral lipid selected from phospholipids; and
optionally, (4) PEGylated lipid;
the lipids constituting the lipid nanoparticles containing calcium ions in a non-precipitated state include one of or a combination of the following components: ionizable cationic lipid, cholesterol and/or cholesterol ester, neutral lipid, and PEGylated lipid;
preferably, the lipids constituting the lipid nanoparticles containing calcium ions in non-precipitated state include the following lipids:
(1) cholesterol lipid selected from cholesterol and/or cholesterol esters;
(2) neutral lipid selected from phospholipids, fatty acid glycerides, or glycolipids, or any combination thereof; and
optionally, (3) PEGylated lipid, and (4) cationic lipid selected from ionizable cationic lipids.

Aspect 33. The calcium-containing cationic lipid nanoparticle composition according to any one of aspects 24-32, wherein the lipids constituting the whole composition include a combination of the following components in % mole:
(1) cationic lipid: 1-90%,
(2) cholesterol lipid: 1-90%,
(3) neutral lipid: 1-90%, and
(4) PEGylated lipid: 0.1-20%.

In some embodiments, (1) the molar fraction of the cationic lipid is 1-20%, 20-40%, 40-60%, 60-75%, or 75-90%;
in some embodiments, (2) the molar fraction of the cholesterol lipid is 1-20%, 20-40%, 40-60%, 60-75%, or 75-90%;
in some embodiments, (3) the molar fraction of the neutral lipid is 1-20%, 20-40%, 40-60%, 60-75%, or 75-90%; and
in some embodiments, (4) the molar fraction of the PEGylated lipid is 1-5%, 5-10%, 10-15%, or 15-20%.

The precondition is that the sum of the percentages of the substances that make up the composition is equal to 100%.

Aspect 34. The calcium-containing cationic lipid nanoparticle composition according to any one of aspects 24-33, wherein the cationic lipid is selected from ionizable cationic lipids; preferably, the ionizable cationic lipid is selected from DSDMA, DLinDMA, DLenDMA, DODMA, A6, OF-02, A18-Iso5-2DC18, 98N₁₂-5, 9A1P9, C12-200, cKK-E12, 7C1, G0-C14, L319, 304O₁₃, OF-Deg-Lin, 306-O12B, 306Oᵢ₁₀, FTT5, SM102, ALC-0315, A9, Lipid 2,2(8,8)4CCH3, CL1, LP01, DLin-MC3-DMA, or analogs thereof;
the neutral phospholipid is selected from one or more of egg yolk lecithin, soybean lecithin, hydrogenated soybean lecithin, phosphatidylcholine, phosphatidylethanolamine, phosphatidylinositol, distearoylphosphatidylcholine, dimyristoylphosphatidylcholine, dipalmitoylphosphatidylcholine, dioleoylphosphatidylcholine, distearoylphosphatidylethanolamine, dimyristoylphosphatidylethanolamine, dipalmitoylphosphatidylethanolamine, dioleoylphosphatidylethanolamine, distearoylphosphatidylinositol, dimyristoylphosphatidylinositol, dipalmitoylphosphatidylinositol, dioleoylphosphatidylinositol, 9A1P9, and 10A1P10; preferably one or more of phosphatidylcholine, egg yolk lecithin, soybean lecithin, hydrogenated soybean lecithin, and phosphatidylethanolamine;
the PEGylated lipid is selected from methoxy polyethylene glycol-distearoylphosphatidylethanolamine (mPEG-DSPE), methoxy polyethylene glycol-dioleoylphosphatidylethanolamine (mPEG-DOPE), methoxy polyethylene glycol-dipalmitoylphosphatidylethanolamine (mPEG-DPPE), polyethylene glycol-dilauroylglycerol (PEG-DAG), polyethylene glycol-dimyristoylglycerol (PEG-DMG), polyethylene glycol-dipalmitoylglycerol (PEG-DPG), polyethylene glycol-distearoylglycerol (PEG-DSG), polyethylene glycol-dioleoylglycerol (PEG-DOG), polyethylene glycol-dilinoleylglycerol (PEG-DLinG), polyethylene glycol-dilauroylpropylamine (PEG-DAA), polyethylene glycol-dimyristoylpropylamine (PEG-DMA), glycol-dipalmitoylpropylamine (PEG-DPA), polyethylene glycol-dioleoylpropylamine polyethylene (PEG-DOA), polyethylene glycol-dilinoleylpropylamine (PEG-DLinA), polyethylene glycol-ceramide (PEG-ceramide), stearoyl polyethylene glycol ester, vitamin E polyethylene glycol succinate (TPGS), and any combination thereof;
wherein the PEG is a PEG group with a degree of polymerization selected from 3-100; preferably, the PEG is a PEG group with a degree of polymerization selected from 3-50 or 50-100; more preferably, the PEG is a PEG group with a degree of polymerization selected from 3-10, 10-20, 20-30, 30-40, 40-50, 50-60, 60-70, 70-80, 80-90, or 90-100; more preferably, the PEG is a PEG group with a degree of polymerization selected from about 5, about 10, about 15, about 20, about 25, about 30, about 35, about 40, about 45, about 50, about 55, about 60, about 65, about 70, about 75, about 80, about 85, about 90, about 95, or about 100.

Aspect 35. The calcium-containing cationic lipid nanoparticle composition according to any one of aspects 24-34, wherein the particle size of the lipid nanoparticle is 25-1000 nm; preferably, the particle size of the lipid nanoparticle is 25-500 nm or 500-1000 nm; more preferably, the particle size of the lipid nanoparticle is 25-75 nm, 75-125 nm, 125-175 nm, 175-225 nm, 225-275 nm, 275-350 nm, 350-500 nm, 500-800 nm, or 800-1000 nm; more preferably, the particle size of the lipid nanoparticle is 40±10 nm, 50±10 nm, 60±10 nm, 70±10 nm, 80±10 nm, 90=10 nm, 100±10 nm, 110±10 nm, 120±10 nm, 125±10 nm, 130±10 nm, 140±10 nm, 150=10 nm, 160±10 nm, 170±10 nm, 180±10 nm, 190±10 nm, 200±10 nm, 210±10 nm, 220±10 nm, or 250±10 nm.

Aspect 36. The calcium-containing cationic lipid nanoparticle composition according to any one of aspects 24-35, wherein the nucleic acid encapsulation efficiency of the lipid nanoparticle is >30%; preferably >40%; preferably >50%; preferably >60%; preferably >70%; preferably >80%; preferably >90%; more preferably >95%; more preferably >97%; more preferably >98%.

Aspect 37. A method for preparing the nucleic acid-loaded calcium-containing cationic lipid nanoparticle according to aspects 1-23 or the calcium-containing cationic lipid nanoparticle composition according to any one of aspects 24-35, wherein the method comprises the following step:
mixing aqueous phase containing a water-soluble calcium salt with organic phase containing a lipid to produce calcium-containing lipid nanoparticles, wherein
preferably, the solvent of the organic phase is selected from solvents that are miscible with water; more preferably, the solvent of the organic phase is selected from methanol, ethanol, n-propanol, isopropanol, n-butanol, isobutanol, tert-butanol, sec-butanol, DMSO, DMF, acetic acid, and any combination thereof.

Aspect 38. The preparation method according to aspect 37, wherein the methods for mixing aqueous phase with organic phase include:
(A1) directly adding the organic phase and the aqueous phase to the same container for mixing;
(A2) allowing the organic phase and the aqueous phase to flow through the same pipeline for mixing; preferably, a mixer of the same pipeline is a tee pipe, a microfluidic chip, or any variant thereof; more preferably, the mixer in the same pipeline is a Y-tube, a T-tube, a microfluidic chip, or any variant thereof; or
a combination of the two mixing methods A1 and A2 defined previously.

Aspect 39. The preparation method according to aspect 37 or 38, wherein the method comprises the following step: the aqueous phase also comprises the nucleic acid to be loaded.

Aspect 40. A method for preparing the nucleic acid-loaded calcium-containing cationic lipid nanoparticle according to aspects 1-23 or the calcium-containing cationic lipid nanoparticle composition according to any one of aspects 24-35, wherein the method comprises the following steps:
(1) mixing lipid organic solution I containing a cationic lipid with aqueous phase I to form intermediate al; and
(2) mixing intermediate al with aqueous phase II containing soluble calcium ions to form calcium-containing cationic lipid nanoparticles.

Aspect 41. The preparation method according to aspect 40, wherein the mixing method in step (1) or step (2) is:
(B1) directly adding the two liquids to the same container for mixing;
(B2) allowing the two liquids to flow through the same pipeline for mixing; preferably, a mixer of the same pipeline is a tee pipe, a microfluidic chip, or any variant thereof; more preferably, the mixer in the same pipeline is a Y-tube, a T-tube, a microfluidic chip, or any variant thereof; or
a combination of the two mixing methods B1 and B2 defined previously; and
preferably, method B2 is used for mixing in step (2).

Aspect 42. The preparation method according to aspect 40 or 41, wherein the method comprises the following step: comprising the nucleic acid to be loaded in aqueous phase I or aqueous phase II.

Aspect 43. A preparation method for preparing the nucleic acid-loaded calcium-containing cationic lipid nanoparticle according to aspects 1-23 or the calcium-containing cationic lipid nanoparticle composition according to any one of aspects 24-35, wherein the preparation method comprises the following steps:
(1) mixing lipid organic solution I containing cationic lipid with aqueous phase I to form intermediate b1;
(2) forming liposome intermediate b2 containing calcium ions in an internal aqueous phase by means of active encapsulation or passive encapsulation; and
(3) mixing intermediate b1 with intermediate b2 to form calcium-containing cationic lipid nanoparticles.

Aspect 44. The preparation method according to aspect 43, wherein the mixing method in step (1), step (2), or step (3) is:
(C1) directly adding the two liquids to the same container for mixing;
(C2) allowing the two liquids to flow through the same pipeline for mixing; preferably, a mixer of the same pipeline is a tee pipe, a microfluidic chip, or any variant thereof; more preferably, the mixer in the same pipeline is a Y-tube, a T-tube, a microfluidic chip, or any variant thereof; or
a combination of the two mixing methods C1 and C2.

Aspect 45. The preparation method according to aspect 43 or 44, wherein the preparation method comprises the following step: comprising the nucleic acid to be loaded in aqueous phase I or aqueous phase II.

Aspect 46. The preparation method according to any one of aspects 37-45, wherein the preparation method further comprises the following step: dialyzing the obtained calcium-containing cationic lipid nanoparticles in a buffer to remove part or all of calcium ions outside the cationic lipid nanoparticles.

Aspect 47. The preparation method according to any one of aspects 37-45, wherein the calcium ions are derived from calcium salts, preferably soluble calcium salts, more preferably calcium acetate, calcium chloride, calcium sodium EDTA, calcium gluconate, calcium dihydrogen phosphate, calcium nitrate, calcium bicarbonate, calcium bisulfate, calcium bisulfite, calcium bromide, calcium iodide, calcium citrate, calcium lactate, and calcium gluconate, further more preferably calcium acetate, calcium sodium EDTA, calcium gluconate, calcium citrate, calcium lactate, and calcium gluconate, further more preferably calcium acetate.

Aspect 48. The preparation method according to any one of aspects 37-45, wherein the calcium ions are derived from a calcium salt solution with a concentration of 50-1000 mmol/L; preferably, the calcium ions are derived from a calcium salt solution with a concentration of 50-150 mmol/L, a calcium salt solution with a concentration of 150-300 mmol/L, a calcium salt solution with a concentration of 300-500 mmol/L, or a calcium salt solution with a concentration of 500-800 mmol/L;
more preferably, the calcium ions are derived from a calcium salt solution with a concentration of 100±50 mmol/L, a calcium salt solution with a concentration of 200±50 mmol/L, a calcium salt solution with a concentration of 300±50 mmol/L, a calcium salt solution with a concentration of 400±50 mmol/L, a calcium salt solution with a concentration of 500±50 mmol/L, a calcium salt solution with a concentration of 600±50 mmol/L, a calcium salt solution with a concentration of 700±50 mmol/L, a calcium salt solution with a concentration of 800±50 mmol/L, or a calcium salt solution with a concentration of 900±50 mmol/L.

Aspect 49. A transfection kit, wherein the kit comprises the nucleic acid-loaded calcium-containing cationic lipid nanoparticles according to any one of aspects 1-23 or the calcium-containing cationic lipid nanoparticle composition according to any one of aspects 24-35.

Aspect 50. Use of the nucleic acid-loaded calcium-containing cationic lipid nanoparticle according to any one of aspects 1-23 or the calcium-containing cationic lipid nanoparticle composition according to any one of aspects 24-35 for gene transfection to cultured cells in vitro.

Aspect 51. The use according to aspect 50, wherein the use is for non-therapeutic purpose, and preferably, the use is for cell modification in vitro.

Aspect 52. Use of the nucleic acid-loaded calcium-containing cationic lipid nanoparticle according to any one of aspects 1-23 or the calcium-containing cationic lipid nanoparticle composition according to any one of aspects 24-35 for local injection into the body to achieve gene transfection.

Aspect 53. Use of the nucleic acid-loaded calcium-containing cationic lipid nanoparticle according to any one of aspects 1-23 or the calcium-containing cationic lipid nanoparticle composition according to any one of aspects 24-35 for the preparation of a gene-based drug for local injection into the body.

Aspect 54. Use of the nucleic acid-loaded calcium-containing cationic lipid nanoparticle according to any one of aspects 1-23 or the calcium-containing cationic lipid nanoparticle composition according to any one of aspects 24-35 for local or systemic injection into the body to achieve vaccine immunization.

Aspect 55. Use of the nucleic acid-loaded calcium-containing cationic lipid nanoparticle according to any one of aspects 1-23 or the calcium-containing cationic lipid nanoparticle composition according to any one of aspects 24-35 for the preparation of a nucleic acid vaccine for local or systemic injection into the body.

In one embodiment, the present invention relates to a calcium-containing cationic lipid nanoparticle for loading with a nucleic acid, wherein
the cationic lipid nanoparticle comprises cationic lipid, neutral lipid, PEGylated lipid, and cholesterol and/or cholesterol ester;
the cationic lipid nanoparticle contains calcium ions, and anions corresponding to the calcium ions are not phosphate radicals, hydrogen phosphate radicals, and dihydrogen phosphate radicals; and the cationic lipid nanoparticle is free of a non-PEG-group-modified negatively charged lipid.

In one embodiment, the calcium-containing cationic lipid nanoparticle for loading with a nucleic acid according to the present invention contains calcium ions in a non-precipitated state.

The calcium-containing cationic lipid nanoparticle according to any one of the preceding embodiments, wherein the concentration of calcium in the whole formulation is 0.01-150 mmol/L; preferably 0.01-0.1 mmol/L, or 0.1-150 mmol/L; preferably 0.01-0.1 mmol/L, 0.1-1 mmol/L, 1-10 mmol/L, 10-100 mmol/L, or 100-150 mmol/L; more preferably 0.01-0.1 mmol/L, 0.1-1 mmol/L, 1-10 mmol/L, 10-30 mmol/L, 30-50 mmol/L, 50-70 mmol/L, 70-90 mmol/L, 90-110 mmol/L, 110-130 mmol/L, or 130-150 mmol/L; more preferably 0.01-1 mmol/L; more preferably 0.02-0.8 mmol/L; more preferably 0.03-0.5 mmol/L; more preferably 0.1-0.5 mmol/L.

The calcium-containing cationic lipid nanoparticle according to any one of the preceding embodiments, wherein the local concentration of calcium in the inner core of the cationic lipid nanoparticle relative to the total volume is 10-300 μM, preferably 15-250 μM, more preferably 20-180 μM, more preferably 20-180 μM, more preferably 60-150 μM.

Preferably, the term “calcium in the cationic lipid nanoparticle” refers to the calcium which is in the whole formulation minus the calcium which is not in the cationic lipid nanoparticles in the formulation.

The calcium-containing cationic lipid nanoparticle according to any one of the preceding embodiments, wherein the molar ratio of calcium to lipid in the cationic lipid nanoparticle is 1:(0.01-20), preferably 1:(0.1-10), preferably 1:(1-10), preferably 1:(0.1-1); more preferably 1:(2-18), more preferably 1:(5-15), more preferably 1:(7-13).

The calcium-containing cationic lipid nanoparticle according to aspect 1 or 2, wherein the molar ratio of the calcium in the cationic lipid nanoparticle to the total amount of the cholesterol and cholesterol ester in a formulation is (0.01:1)-(0.8:1); preferably (0.02:1)-(0.6:1); more preferably (0.03:1)-(0.4:1); more preferably (0.08:1)-(0.3:1).

The calcium-containing cationic lipid nanoparticle according to any one of the preceding embodiments, wherein the calcium ions are derived from calcium salts, preferably soluble calcium salts, more preferably calcium acetate, calcium chloride, calcium sodium EDTA, calcium gluconate, calcium dihydrogen phosphate, calcium nitrate, calcium bicarbonate, calcium bisulfate, calcium bisulfite, calcium bromide, calcium iodide, calcium citrate, calcium lactate, and calcium gluconate, further more preferably calcium acetate, calcium sodium EDTA, calcium gluconate, calcium citrate, calcium lactate, and calcium gluconate, further more preferably calcium acetate;
preferably, the calcium ions are derived from a calcium salt solution with a concentration of 50-1000 mmol/L; preferably, the calcium ions are derived from a calcium salt solution with a concentration of 50-150 mmol/L, a calcium salt solution with a concentration of 150-300 mmol/L, a calcium salt solution with a concentration of 300-500 mmol/L, or a calcium salt solution with a concentration of 500-800 mmol/L;
more preferably, the calcium ions are derived from a calcium salt solution with a concentration of 100±50 mmol/L, a calcium salt solution with a concentration of 200±50 mmol/L, a calcium salt solution with a concentration of 300±50 mmol/L, a calcium salt solution with a concentration of 400±50 mmol/L, a calcium salt solution with a concentration of 500±50 mmol/L, a calcium salt solution with a concentration of 600±50 mmol/L, a calcium salt solution with a concentration of 700±50 mmol/L, a calcium salt solution with a concentration of 800±50 mmol/L, or a calcium salt solution with a concentration of 900±50 mmol/L.

The calcium-containing cationic lipid nanoparticle according to any one of the preceding embodiments, wherein a substance to be delivered by the calcium-containing cationic lipid nanoparticle is a nucleic acid, preferably plasmid DNA, single-stranded DNA, double-stranded DNA, siRNA, shRNA, aiRNA, miRNA, mRNA, circular RNA, tRNA, rRNA, vRNA, gRNA, aptamer, ribozyme, oligonucleotide, or any combination thereof.

The calcium-containing cationic lipid nanoparticle according to any one of the preceding embodiments, wherein the molar number of phosphate radicals in the nucleic acid:the molar number of positive charges in the cationic lipid is 1:(0.5-20), preferably 1:(1-10), more preferably 1:(1.5-6), more preferably 1:(1.5-3) or 1:(3-6).

The calcium-containing cationic lipid nanoparticle according to any one of the preceding embodiments, wherein the mass ratio of the nucleic acid to the lipid is 1:(1-100), preferably 1:(5-90), more preferably 1:(10-70), further preferably 1:(10-30).

The calcium-containing cationic lipid nanoparticle according to any one of the preceding embodiments, wherein the length of the substance to be delivered by the calcium-containing cationic lipid nanoparticle is about 15-30000 bases (base pairs); preferably 15-60, 60-120, 120-250, 250-500, 500-1000, 1000-2000, 2000-4000, 4000-8000, 8000-15000, 15000-20000, 20000-25000, or 25000-30000 bases (base pairs); more preferably 15-60, 15-50, 15-40, 15-30, 15-25, 19-25, 20-30, 20-50, 20-80, 30-50, 30-80, 30-120, 50-100, 50-150, 50-250, 100-200, 100-300, 100-500, 200-500, 200-1000, 300-800, 300-1500, 1000-3000, 1000-5000, 1000-8000, 5000-10000, 5000-15000, 5000-20000, 10000-25000, or 10000-30000 bases (base pairs).

The calcium-containing cationic lipid nanoparticle according to any one of the preceding embodiments, wherein the amount of the loaded nucleic acid is 5 μg/ml-10 mg/ml;
preferably, the amount of the loaded nucleic acid is 5-10 μg/ml, 10-20 μg/ml, 20-40 μg/ml, 40-80 μg/ml, 80-150 μg/ml, 150-300 μg/ml, 300-400 μg/ml, 400-800 μg/ml, 800 μg/ml-1 mg/ml, 1-1.5 mg/ml, 1.5-2 mg/ml, 2-4 mg/ml, 4-6 mg/ml, 6-8 mg/ml, or 8-10 mg/ml;
more preferably, the amount of the loaded nucleic acid is 50±50 μg/ml, 100±50 μg/ml, 200±50 μg/ml, 300±50 μg/ml, 400±50 μg/ml, 500±50 μg/ml, 600±50 μg/ml, 700±50 μg/ml, 800±50 μg/ml, 900±50 μg/ml, 1000±50 μg/ml, 1500±50 μg/ml, 2000±50 μg/ml, 2500±50 μg/ml, 3000±50 μg/ml, 4000±50 μg/ml, 5000±50 μg/ml, 6000±50 μg/ml, 7000±50 μg/ml, 8000±50 μg/ml, 9000±50 μg/ml, or 10000±50 μg/ml.

The calcium-containing cationic lipid nanoparticle according to any one of the preceding embodiments, wherein the lipids constituting the cationic lipid nanoparticle comprise any one of cationic lipid, cholesterol and/or cholesterol ester, neutral lipid, and PEGylated lipid, or the combination thereof;
preferably, the lipids constituting the cationic lipid nanoparticle comprise the following lipids:
(1) cationic lipid selected from ionizable cationic lipids;
(2) cholesterol lipid selected from cholesterol and/or cholesterol esters;
(3) neutral lipid selected from phospholipids, fatty acid glycerides, or glycolipids, or any combination thereof;
and
optionally, (4) PEGylated lipid;
more preferably, the lipids constituting the cationic lipid nanoparticle comprise:
(1) cationic lipid selected from ionizable cationic lipids;
(2) cholesterol lipid selected from cholesterol;
(3) neutral lipid selected from phospholipids; and
optionally, (4) PEGylated lipid.

The calcium-containing cationic lipid nanoparticle according to any one of the preceding embodiments, wherein
the lipids constituting the cationic lipid nanoparticle include 1-90% by mole of cationic lipid and/or 1-90% by mole of cholesterol lipid;
preferably, the lipids constituting the cationic lipid nanoparticle include 10-60% by mole of cationic lipid and/or 25-75% by mole of cholesterol lipid;
more preferably, the lipids constituting the cationic lipid nanoparticle include 20-40% by mole of cationic lipid and/or 40-60% by mole of cholesterol lipid.

The calcium-containing cationic lipid nanoparticle according to any one of the preceding embodiments, wherein
the lipids constituting the cationic lipid nanoparticle include a combination of the following components in % mole:
(1) cationic lipid: 1-90%,
(2) cholesterol lipid: 1-90%,
(3) neutral lipid: 1-90%, and
(4) PEGylated lipid: 0.1-20%;
preferably, the lipids constituting the cationic lipid nanoparticle include a combination of the following components in % mole:
(1) cationic lipid: 10-50%,
(2) cholesterol lipid: 25-75%,
(3) neutral lipid: 1-30%, and
(4) PEGylated lipid: 0.5-10%;
more preferably, the lipids constituting the cationic lipid nanoparticle include a combination of the following components in % mole:
(1) cationic lipid: 20-40%,
(2) cholesterol lipid: 40-60%,
(3) neutral lipid: 5-25%, and
(4) PEGylated lipid: 1-3%.

The calcium-containing cationic lipid nanoparticle according to any one of the preceding embodiments, wherein the cationic lipid nanoparticle is free of a non-PEG-group-modified negatively charged lipid.

The calcium-containing cationic lipid nanoparticle according to any one of the preceding examples, wherein the cationic lipid is selected from ionizable cationic lipids; preferably, the ionizable cationic lipid is selected from DSDMA, DLinDMA, DLenDMA, DODMA, A6, OF-02, A18-Iso5-2DC18, 98N₁₂-5, 9A1P9, C12-200, cKK-E12, 7C1, G0-C14, L319, 304O₁₃, OF-Deg-Lin, 306-O12B, 306Oᵢ₁₀, FTT5, SM102, ALC-0315, A9, Lipid 2,2(8,8)4CCH3, CL1, LP01, DLin-MC3-DMA, analogs thereof or combinations thereof; and/or
the neutral phospholipid is selected from one or more of egg yolk lecithin, soybean lecithin, hydrogenated soybean lecithin, phosphatidylcholine, phosphatidylethanolamine, phosphatidylinositol, distearoylphosphatidylcholine, dimyristoylphosphatidylcholine, dipalmitoylphosphatidylcholine, dioleoylphosphatidylcholine, distearoylphosphatidylethanolamine, dimyristoylphosphatidylethanolamine, dipalmitoylphosphatidylethanolamine, dioleoylphosphatidylethanolamine, distearoylphosphatidylinositol, dimyristoylphosphatidylinositol, dipalmitoylphosphatidylinositol, dioleoylphosphatidylinositol, 9A1P9, and 10A1P10; preferably one or more of phosphatidylcholine, egg yolk lecithin, soybean lecithin, hydrogenated soybean lecithin, and phosphatidylethanolamine; and/or
the PEGylated lipid is selected from methoxy polyethylene glycol-distearoylphosphatidylethanolamine (mPEG-DSPE), methoxy polyethylene glycol-dioleoylphosphatidylethanolamine (mPEG-DOPE), methoxy polyethylene glycol-dipalmitoylphosphatidylethanolamine (mPEG-DPPE), polyethylene glycol-dilauroylglycerol (PEG-DAG), polyethylene glycol-dimyristoylglycerol (PEG-DMG), polyethylene glycol-dipalmitoylglycerol (PEG-DPG), polyethylene glycol-distearoylglycerol (PEG-DSG), polyethylene glycol-dioleoylglycerol (PEG-DOG), polyethylene glycol-dilinoleylglycerol (PEG-DLinG), polyethylene glycol-dilauroylpropylamine (PEG-DAA), polyethylene glycol-dimyristoylpropylamine (PEG-DMA), polyethylene glycol-dipalmitoylpropylamine (PEG-DPA), polyethylene glycol-dioleoylpropylamine (PEG-DOA), polyethylene glycol-dilinoleylpropylamine (PEG-DLinA), polyethylene glycol-ceramide (PEG-ceramide), stearoyl polyethylene glycol ester, vitamin E polyethylene glycol succinate (TPGS), and any combination thereof;
wherein the PEG is a PEG group with a degree of polymerization selected from 3-100; preferably, the PEG is a PEG group with a degree of polymerization selected from 3-50 or 50-100; more preferably, the PEG is a PEG group with a degree of polymerization selected from 3-10, 10-20, 20-30, 30-40, 40-50, 50-60, 60-70, 70-80, 80-90, or 90-100; more preferably, the PEG is a PEG group with a degree of polymerization selected from about 5, about 10, about 15, about 20, about 25, about 30, about 35, about 40, about 45, about 50, about 55, about 60, about 65, about 70, about 75, about 80, about 85, about 90, about 95, or about 100.

The calcium-containing cationic lipid nanoparticle according to any one of the preceding embodiments, wherein the particle size of the calcium-containing cationic lipid nanoparticle is 25-1000 nm; preferably, the particle size of the lipid nanoparticle is 25-500 nm or 500-1000 nm; more preferably, the particle size of the lipid nanoparticle is 25-75 nm, 75-125 nm, 125-175 nm, 175-225 nm, 225-275 nm, 275-350 nm, 350-500 nm, 500-800 nm, or 800-1000 nm; more preferably, the particle size of the lipid nanoparticle is 40±10 nm, 50±10 nm, 60±10 nm, 70±10 nm, 80±10 nm, 90±10 nm, 100±10 nm, 110±10 nm, 120±10 nm, 125±10 nm, 130±10 nm, 140±10 nm, 150±10 nm, 160±10 nm, 170±10 nm, 180±10 nm, 190=10 nm, 200±10 nm, 210±10 nm, 220±10 nm, or 250±10 nm.

The calcium-containing cationic lipid nanoparticle according to any one of the preceding embodiments, wherein the calcium-containing cationic lipid nanoparticle is selected from the following nanoparticle formulations:
an siRNA-loaded calcium-containing cationic lipid nanoparticle, with a cationic lipid (preferably DLin-MC3-DMA), cholesterol, a neutral phospholipid (preferably DSPC), and a PEGylated lipid (preferably PEG2000-DMG) as lipids and calcium acetate as a calcium ion source;
an mRNA-loaded calcium-containing cationic lipid nanoparticle, with a cationic lipid (preferably DLin-MC3-DMA), cholesterol, a neutral phospholipid (preferably DSPC), and a PEGylated lipid (preferably PEG2000-DMG) as lipids and calcium acetate as a calcium ion source; and
a DNA-loaded calcium-containing cationic lipid nanoparticle, with a cationic lipid (preferably DLin-MC3-DMA), cholesterol, a neutral phospholipid (preferably DSPC), and a PEGylated lipid (preferably PEG2000-DMG) as lipids and calcium acetate as a calcium ion source.

The calcium-containing cationic lipid nanoparticle according to any one of the proceeding embodiments, wherein the calcium-containing cationic lipid nanoparticle has targeting effects on liver, lung, or spleen.

In one embodiment, the present invention relates to a calcium-containing cationic lipid nanoparticle composition, wherein the calcium-containing cationic lipid nanoparticle composition is prepared by mixing calcium-containing cationic lipid nanoparticles with nucleic acid-loaded cationic lipid nanoparticles, and preferably after mixing, adjusting the pH value to neutrality.

In one embodiment, the present invention relates to the use of the nucleic acid-loaded calcium-containing cationic lipid nanoparticle or the calcium-containing cationic lipid nanoparticle composition for gene transfection to cultured cells in vitro. In one embodiment, the use is for non-therapeutic purpose, and preferably, the use is for cell modification in vitro.

In one embodiment, the present invention relates to the use of the nucleic acid-loaded calcium-containing cationic lipid nanoparticle or the calcium-containing cationic lipid nanoparticle composition for local injection into the body to achieve gene transfection.

In one embodiment, the present invention relates to the use of the nucleic acid-loaded calcium-containing cationic lipid nanoparticle or the calcium-containing cationic lipid nanoparticle composition for the formulation of a gene-based drug for local injection into the body.

In one embodiment, the present invention relates to the use of the nucleic acid-loaded calcium-containing cationic lipid nanoparticle or the calcium-containing cationic lipid nanoparticle composition for local or systemic injection into the body to achieve vaccine immunization.

In one embodiment, the present invention relates to the use of the nucleic acid-loaded calcium-containing cationic lipid nanoparticle or the calcium-containing cationic lipid nanoparticle composition for the formulation of a nucleic acid vaccine for local or systemic injection into the body.

A method for preparing the calcium-containing cationic lipid nanoparticle composition according to any one of the preceding embodiments, characterized by comprising the following step:
mixing an aqueous phase containing a water-soluble calcium salt with an organic phase containing a lipid to produce calcium-containing lipid nanoparticles;
preferably, the solvent in the organic phase is selected from solvents that are miscible with water; more preferably, the solvent in the organic phase is selected from methanol, ethanol, n-propanol, isopropanol, n-butanol, isobutanol, tert-butanol, sec-butanol, DMSO, DMF, acetic acid, and any combination thereof.

Preferably, in the preparation method, methods for mixing the aqueous phase with the organic phase include:
(A1) directly adding the organic phase and the aqueous phase to the same container for mixing;
(A2) allowing the organic phase and the aqueous phase to flow through the same pipeline for mixing; preferably, a mixer of the same pipeline is a tee pipe, a microfluidic chip, or any variant thereof; more preferably, the mixer in the same pipeline is a Y-tube, a T-tube, a microfluidic chip, or any variant thereof; or a combination of the two mixing methods A1 and A2 defined previously.

Preferably, the method comprises the following step: also comprising the nucleic acid to be loaded in the aqueous phase.

The preparation method according to any one of the preceding examples, wherein the method comprises the following steps:
(1) mixing lipid organic solution I containing a cationic lipid with aqueous phase I to form intermediate al; and
(2) mixing intermediate al with aqueous phase II containing soluble calcium ions to form calcium-containing cationic lipid nanoparticles.

Preferably, in the preparation method, the mixing method in step (1) or step (2) is:
(B1) directly adding the two liquids to the same container for mixing;
(B2) allowing the two liquids to flow through the same pipeline for mixing; preferably, a mixer of the same pipeline is a tee pipe, a microfluidic chip, or any variant thereof; more preferably, the mixer in the same pipeline is a Y-tube, a T-tube, a microfluidic chip, or any variant thereof; or a combination of the two mixing methods B1 and B2 defined previously; and
preferably, in step (2), method B2 is used for mixing.

The preparation method according to any one of the preceding embodiments, wherein the preparation method comprises the following step: comprising the nucleic acid to be loaded in aqueous phase I or aqueous phase II.

A method for preparing the calcium-containing cationic lipid nanoparticle composition according to any one of the preceding embodiments, wherein the method comprises the following step:
(1) mixing lipid organic solution I containing a cationic lipid with aqueous phase I to form intermediate b1;
(2) forming liposome intermediate b2 containing calcium ions in an internal aqueous phase by means of active encapsulation or passive encapsulation; and
(3) mixing intermediate b1 with intermediate b2 to form calcium-containing cationic lipid nanoparticles.

Preferably, in the preparation method, the mixing method in step (1), step (2), or step (3) is:
(C1) directly adding the two liquids to the same container for mixing;
(C2) allowing the two liquids to flow through the same pipeline for mixing; preferably, a mixer of the same pipeline is a tee pipe, a microfluidic chip, or any variant thereof; more preferably, the mixer in the same pipeline is a Y-tube, a T-tube, a microfluidic chip, or any variant thereof; or
a combination of the two mixing methods C1 and C2.

Preferably, the preparation method comprises the following step: comprising the nucleic acid to be loaded in aqueous phase I or aqueous phase II.

The preparation method according to any one of the preceding embodiments, wherein the method further comprises the following step: dialyzing the obtained calcium-containing cationic lipid nanoparticles in a buffer to remove part or all of calcium ions outside the cationic lipid nanoparticles.

The preparation method according to any one of the preceding embodiments, wherein the calcium ions are derived from calcium salts, preferably soluble calcium salts, more preferably calcium acetate, calcium chloride, calcium sodium EDTA, calcium gluconate, calcium dihydrogen phosphate, calcium nitrate, calcium bicarbonate, calcium bisulfate, calcium bisulfite, calcium bromide, calcium iodide, calcium citrate, calcium lactate, and calcium gluconate, further more preferably calcium acetate, calcium sodium EDTA, calcium gluconate, calcium citrate, calcium lactate, and calcium gluconate, further more preferably calcium acetate.

The preparation method according to any one of the preceding embodiments, wherein the calcium ions are derived from a calcium salt solution with a concentration of 50-1000 mmol/L; preferably, the calcium ions are derived from a calcium salt solution with a concentration of 50-150 mmol/L, a calcium salt solution with a concentration of 150-300 mmol/L, a calcium salt solution with a concentration of 300-500 mmol/L, or a calcium salt solution with a concentration of 500-800 mmol/L;

more preferably, the calcium ions are derived from a calcium salt solution with a concentration of 100±50 mmol/L, a calcium salt solution with a concentration of 200±50 mmol/L, a calcium salt solution with a concentration of 300±50 mmol/L, a calcium salt solution with a concentration of 400±50 mmol/L, a calcium salt solution with a concentration of 500±50 mmol/L, a calcium salt solution with a concentration of 600±50 mmol/L, a calcium salt solution with a concentration of 700±50 mmol/L, a calcium salt solution with a concentration of 800±50 mmol/L, or a calcium salt solution with a concentration of 900±50 mmol/L.

A transfection kit, wherein the kid comprises the nucleic acid-loaded calcium-containing cationic lipid nanoparticle or the calcium-containing cationic lipid nanoparticle composition according to any one of the preceding embodiments.

An organic phase in which a lipid is dissolved is mixed with a calcium-containing aqueous phase to form calcium-containing cationic lipid nanoparticles. Based on the research, the inventors have surprisingly found that when the inner core of the cationic lipid nanoparticle contains a soluble calcium salt, using the lipid nanoparticle to load a nucleic acid drug results in the properties of high transfection efficiency, stable encapsulation, uniform and controllable particle size, and specific targeting to liver organs. Through an experiment in which the nucleic acid was detected effective, it has been found that the interfering RNA loaded by the calcium-containing cationic lipid nanoparticle of the present invention can significantly knock down the content of the related mRNA, and the loaded mRNA can significantly increase the amount of the expressed related protein. For the most challenging DNA in the field of gene transfection, it can also be transfected into the nucleus to achieve the expression of a large amount of the related protein.

Unless otherwise indicated, the following terms have their assigned meanings.

Unless the context requires otherwise, the word “include” and its variants in the description and claims, such as “including” and “includes”, are interpreted in an open and inclusive sense, i.e., “including but not limited to”.

The term “calcium ions in a precipitated state” refers to a calcium salt, the pure substance of which is hardly soluble in the conventional state, e.g., calcium phosphate, calcium carbonate, or calcium fluoride. The term “calcium ions in a non-precipitated state” means that its pure substance is not hardly soluble in the normal state, that is, they include soluble, easily soluble and slightly soluble calcium ions, including but not limited to calcium acetate, calcium chloride, calcium sodium EDTA, calcium gluconate, calcium dihydrogen phosphate, calcium nitrate, calcium bicarbonate, calcium bisulfate, calcium bisulfite, calcium bromide, calcium iodide, calcium citrate, calcium lactate, calcium gluconate, etc.

The term “DNA” refers to use a non-replicating gene or replicating gene vector as an expression vector.

The terms “interfering RNA” or “RNAi” or “interfering RNA sequence” refer to a single-stranded RNA (e.g., mature miRNA) or a double-stranded RNA (i.e., double-stranded RNA, such as siRNA, aiRNA, or pre-miRNA) which can reduce or inhibit the expression of a target gene or sequence (e.g., by mediating degradation and inhibiting the translation of mRNA complementary to the sequence of the interfering RNA) when the interfering RNA is in the same cell as the target gene or sequence. Therefore, interfering RNA refers to a single-stranded RNA complementary to the sequence of the target mRNA or a double-stranded RNA formed by two complementary strands or a single self-complementary strand. The interfering RNA may have a substantial or complete identity to the target gene or sequence, or may include mismatch regions (i.e., mismatch motifs). The sequence of the interfering RNA can correspond to the full-length target gene or a subsequence thereof.

Interfering RNAs include “small interfering RNAs” or “siRNAs”, for example, interfering RNAs with about 15-60, 15-50, or 15-40 (duplex) nucleotides in length, more typically with about 15-30, 15-25, or 19-25 (duplex) nucleotides in length, and preferably with about 20-24, 21-22, or 21-23 (duplex) nucleotides in length (for example, each complementary strand sequence of the double-stranded siRNA has 15-60, 15-50, 15-40, 15-30, 15-25, or 19-25 nucleotides in length, preferably about 20-24, 21-22, or 21-23 nucleotides in length, and the double-stranded siRNA has about 15-60, 15-50, 15-40, 15-30, 15-25, or 19-25 base pairs in length, preferably about 18-22, 19-20, or 19-21 base pairs in length). The siRNA duplex may comprise a 3′ overhang and a 5′ phosphate terminal with about 1 to about 4 nucleotides or about 2 to about 3 nucleotides. Examples of siRNAs include, but are not limited to, a double-stranded polynucleotide molecule assembled by two separate chain molecules, one of which is a sense chain and the other of which is a complementary antisense strand; a double-stranded polynucleotide molecule assembled by a single-stranded molecule, wherein a sense region and an antisense region are connected via a linker based on a nucleic acid or a non-nucleic acid; a double-stranded polynucleotide molecule containing a hairpin secondary structure with a self-complementary sense region and antisense region; and a ring single-stranded polynucleotide molecule containing two or more ring structures and a stem with a self-complementary sense region and antisense region, wherein the ring polynucleotide can be processed in vivo or in vitro to produce an active double-stranded siRNA molecule.

Preferably, siRNA is chemically synthesized. siRNA may also be produced by cleaving a longer dsRNA (for example, a dsRNA with more than about 25 nucleotides in length) using E. coli RNase III or Dicer. These enzymes process dsRNAs into bioactive siRNAs (see, for example, Yang et al., Proc. Natl. Acad. Sci. USA, 99:9942-9947 (2002); Calegari et al., Proc. Natl. Acad. Sci. USA, 99:14236 (2002); Byrom et al., Ambion TechNotes, 10 (1): 4-6 (2003); Kawasaki et al., Nucleic Acids Res., 31:981-987 (2003); Knight et al., Science, 293:2269-2271 (2001); and Robertson et al., J. Biol. Chem., 243:82 (1968)). Preferably, the dsRNA has at least 50 nucleotides to about 100, 200, 300, 400, or 500 nucleotides in length. The dsRNA may have up to 1000, 1500, 2000, or 5000 nucleotides or more in length. The dsRNA can encode a complete gene transcript or a partial gene transcript. In some examples, the siRNA may be encoded by a plasmid (e.g., transcribed into a sequence that automatically folds into a duplex with a hairpin loop).

As used herein, the term “effector cell” refers to a cell, preferably a mammalian cell, that produces a detectable immune response when contacted with immunostimulatory interfering RNA such as unmodified siRNA. Exemplary effector cells include, for example, dendritic cells, macrophages, peripheral blood mononuclear cells (PBMCs), spleen cells, and the like.

The term “substantially the same” or “substantially identical” in the context of two or more nucleic acids means two or more sequences or subsequences that are identical or have a specific percentage of identical nucleotides (i.e., at least about 60%, preferably at least about 65%, 70%, 75%, 80%, 85%, 90%, or 95% identity within a specific region range) during comparison and alignment is made for the maximum correspondence within a comparison window or within a specified region by using one of the following sequence comparison algorithms or by manual alignment and visual measurement. This definition, when indicated in this context, also similarly means a complement to a sequence. Preferably, substantial identity exists within a region range with at least about 5, 10, 15, 20, 25, 30, 35, 40, 45, 50, 55, or 60 nucleotides in length.

As used herein, the term “nucleic acid” refers to a polymer containing at least two deoxyribonucleotides or ribonucleotides in a single-stranded or double-stranded form and includes DNAs and RNAs. The DNAs may be in the following forms: for example, antisense molecules, plasmid DNAs, preconcentrated DNAs, PCR products, vectors (P1, PAC, BAC, YAC, and artificial chromosomes), expression cassettes, chimeric sequences, chromosomal DNAs, or derivatives and combinations of these components. The RNAs may be in the following forms: siRNAs, asymmetric interfering RNAs (aiRNAs), microRNAs (miRNAs), mRNAs, tRNAs, rRNAs, tRNAs, viral RNAs (vRNAs), and combinations thereof. Nucleic acids include nucleic acids comprising known nucleotide analogs or modified backbone residues or bonds, which are synthetic, naturally occurring, and non-naturally occurring, and which have binding properties similar to those of reference nucleic acids. Examples of the analogs include, but are not limited to, thiophosphates, chiral-methylphosphates, and aminophosphates, methylphosphates, 2′-O-methylribonucleotides, peptide-nucleic acids (PNAs). Unless otherwise defined, the term includes nucleic acids comprising known analogs of natural nucleotides that have similar binding properties to reference nucleic acids. Unless otherwise indicated, specific nucleic acid sequences also inherently include conservatively modified variants thereof (e.g., degenerate codon substitutions), alleles, orthologs, SNPs, and complementary sequences, as well as explicitly indicated sequences. In particular, degenerate codon substitution can be realized by generating a sequence in which the third position of one or more selected (or all) codons is replaced by mixed basic and/or deoxyinosine residues (Batzer et al., Nucleic Acid Res., 19: 5081 (1991); Ohtsuka et al., J. Biol. Chem., 260:2605-2608 (1985); Rossolini et al., Mol. Cell. Probes, 8:91-98 (1994)). The term “nucleotide” includes a sugar deoxyribose (DNA) or ribose (RNA), a base, and a phosphate group. Nucleotides are linked together via phosphate groups. The term “base” includes purines and pyrimidines, which further include natural compounds, i.e., adenine, thymine, guanine, cytosine, uracil, inosine, and natural analogues, and synthetic derivatives of purines and pyrimidines, including but not limited to modification by placing new reactive groups such as but not limited to amines, alcohols, thiols, carboxylates, and alkyl halides.

The term “gene” refers to a nucleic acid (e.g., DNA or RNA) sequence that comprises a partial or full-length coding sequence necessary for producing a polypeptide or precursor polypeptide.

The term “lipid” refers to a group of organic compounds, including but not limited to esters of fatty acids, which are characterized by being insoluble in water but soluble in many organic solvents. Generally, they are divided into at least three categories: (1) “simple lipids”, including fats and oils and waxes; (2) “compound lipids”, including phospholipids and glycolipids; and (3) “derived lipids” such as steroids.

As used herein, “lipid particles” refer to a lipid formulation that can be used for delivering an active agent or therapeutic agent, such as a nucleic acid (e.g., interfering RNA), to a target site of interest. In the lipid particles of the present invention, which are typically formed from cationic lipids, non-cationic lipids, and conjugated lipids that prevent particle aggregation, the active agent or therapeutic agent can be encapsulated in the lipid, thereby protecting the agent from enzymatic degradation.

The term “amphiphilic lipid” refers in part to any suitable material, in which a hydrophobic part of the lipid material is directed to a hydrophobic phase and a hydrophilic part is directed to an aqueous phase. The hydrophilicity is derived from the existence of polar or charged groups such as sugar, phosphate, carboxyl, sulfate, amino, sulfhydryl, nitro, hydroxyl and other similar groups. The hydrophobicity may be imparted by comprising nonpolar groups, including, but not limited to, long-chain saturated and unsaturated aliphatic hydrocarbon groups and groups substituted with one or more aromatic, alicyclic or heterocyclic groups. Examples of amphiphilic compounds include, but are not limited to, phospholipids, amino lipids, and sphingolipids.

Representative examples of phospholipids include, but are not limited to, phosphatidylcholine, phosphatidylethanolamine, phosphatidylserine, phosphatidylinositol, phosphatidic acid, palmitoyloleoylphosphatidylcholine, lysophosphatidylcholine, lysophosphatidylethanolamine, dipalmitoylphosphatidylcholine, dioleoylphosphatidylcholine, distearoylphosphatidylcholine, and dilinoleylphosphatidylcholine. Other compounds lacking phosphorus, such as sphingolipids, the glycosphingolipid family, diacylglycerol, and β-acyloxy acids, are also included in the group called amphiphilic lipids. In addition, the above amphipathic lipids can be mixed with other lipids, including triglycerides and sterols.

The term “neutral lipid”, when present in lipid particles, can be any of a number of lipid species that exist in an uncharged or neutral zwitterionic form at a physiological pH. Such lipids include, for example, diacylphosphatidylcholine, diacylphosphatidylethanolamine, ceramide, sphingomyelin, dihydrosphingomyelin, cholesterol, cephalin, and cerebroside. The selection of neutral lipids for the particles described herein is usually guided based on considerations such as the size of liposomes and the stability of liposomes in the blood stream. Preferably, the neutral lipid component is a lipid having two acyl groups (i.e., diacylphosphatidylcholine and diacylphosphatidylethanolamine). Liposomes with various acyl chain groups having different chain lengths and degrees of saturation are available or can be isolated or synthesized by well-known techniques. In one group of embodiments, lipids containing saturated fatty acids having a carbon chain length ranging from C10 to C20 are preferred. In another group of embodiments, lipids with mono- or di-unsaturated fatty acids having a carbon chain length ranging from C10 to C20 are used. In addition, lipids having a mixture of saturated and unsaturated fatty acid chains can be used. Preferably, the neutral lipid used in the present invention is DOPE, DSPC, POPC, DPPC, or any related phosphatidylcholine. The neutral lipid used in the present invention may also be composed of sphingomyelin, dihydrosphingomyelin, or phospholipids with other head groups, such as serine and inositol.

The term “negatively charged lipid” refers to a lipid containing free phosphate hydroxyl groups in the molecule. Due to the existence of the phosphate hydroxyl group, such lipids can be negatively charged by ionizing positively charged hydrogen ions in a solution, including but not limited to acidic phospholipids such as phosphatidylserine.

The term “negatively charged lipid with a PEG group” refers to a PEG-modified negatively charged lipid, including but not limited to distearoylphosphatidylethanolamine-methoxy polyethylene glycol 2000 (DSPE-MPEG2000).

The term “non-PEG-group-modified negatively charged lipid” refers to a negatively charged lipid free of PEG modification, e.g., phosphatidylserine, phosphatidic acid, and phosphatidylglycerol. The term “non-cationic lipid” refers to any amphiphilic lipid and any other neutral or anionic lipid.

The term “anionic lipid” refers to any lipid that is negatively charged at a physiological pH value. Such lipids include, but are not limited to, phosphatidylglycerol, cardiolipin, diacylphosphatidylserine, diacylphosphatidic acid, N-lauroylphosphatidylethanolamine, N-succinylphosphatidylethanolamine, N-glutarylphosphatidylethanolamine, lysylphosphatidylglycerol, palmitoyloleoylphosphatidylglycerol (POPG), and other anionic modification groups linked to neutral lipids.

The term “cationic lipid” refers to any one of a number of lipid species that carry a net positive charge at a selected pH value, such as at a physiological pH value (for example, a pH of about 7). It has been unexpectedly found that cationic lipids comprising alkyl chains with multiple sites of unsaturation, for example, at least 2 or 3 sites of unsaturation, are particularly effective for forming lipid particles with increased membrane fluidity. Many cationic lipids and related analogs that are also effective in the present invention have been described in U.S. Patent Publication Nos. 20060083780 and 20060240554; U.S. Pat. Nos. 5,208,036; 5,264,618; 5,279,833; 5,283,185; 5,753,613; and 5,785,992; and PCT Publication No. WO 96/10390, the disclosures of which are incorporated herein by reference in their entirety for all purposes. Non-limiting examples of cationic lipids are described in detail herein. In some cases, the cationic lipid comprises a protonatable tertiary amine (e.g., pH titratable) head group, a C18 alkyl chain, an ether bond between the head group and the alkyl chain, and 0 to 3 double bonds. Such lipids include, for example, DSDMA, DLinDMA, DLenDMA, DODMA, A6, OF-02, A18-Iso5-2DC18, 98N₁₂-5, 9A1P9, C12-200, cKK-E12, 7C1, G0-C14, L319, 304O₁₃, OF-Deg-Lin, 306-O12B, 306Oᵢ₁₀, FTT5, SM102, ALC-0315, A9, Lipid 2,2(8,8)4CCH3, CL1, LP01, MC3, or analogs thereof.

The term “analog of cationic lipid” refers to a cationic lipid with a similar structure produced by changing the number of carbon atoms in an optional carbon chain of a cationic lipid.

In some embodiments, the cationic lipid is selected from a cationic lipid of formula (Ia):

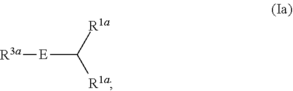

wherein R^{1a} and R^{2a} are each independently at each occurrence C₁₀-C₃₀ alkyl, C₁₀-C₃₀ alkenyl, or C₁₀-C₃₀ alkynyl; preferably, wherein R^{1a} and R^{2a} each comprise at least one site of unsaturation; more preferably, wherein R^{1a} and R^{2a} each comprise at least two sites of unsaturation; more preferably, wherein R^{1a} and R^{2a} each comprise at least two double bonds;
R^{3a} is NH₂—C₁₋₈ alkyl-, NH(C₁₋₈ alkyl)-C₁₋₈ alkyl-, or NH(C₁₋₈ alkyl)₂-C₁₋₈ alkyl-; preferably, R^{3a} is NH₂—(CH₂)—, NH₂—(CH₂)₂—, NH₂—(CH₂)₃—, NH₂—(CH₂)₄—, NH₂—(CH₂)₅—, NH₂—(CH₂)₆—, NH₂—(CH₂)₇—, NH₂—(CH₂)₈—, NH(CH₃)—(CH₂)—, NH(CH₃)—(CH₂)₂—, NH(CH₃)—(CH₂)₃—, NH(CH₃)—(CH₂)₄—, NH(CH₃)—(CH₂)₅—, NH(CH₃)—(CH₂)₆—, NH(CH₃)—(CH₂)₇—, NH(CH₃)—(CH₂)₈—, N(CH₃)₂—(CH₂)—, N(CH₃)₂—(CH₂)₂—, N(CH₃)₂—(CH₂)₃—, N(CH₃)₂—(CH₂)₄—, N(CH₃)₂—(CH₂)₅—, N(CH₃)₂—(CH₂)₆—, N(CH₃)₂—(CH₂)₇—, or N(CH₃)₂—(CH₂)₈—;
E is O, N(Q)C(O), C(O)N(Q), (Q)NC(O)O, OC(O)N(Q), SS, or O═N, and Q is hydrogen or methyl, ethyl, n-propyl, isopropyl, n-butyl, isobutyl, sec-butyl, or tert-butyl; preferably, E is O, NHC(O), N(CH₃)C(O), C(O)NH, C(O)N(CH₃), NHC(O)O, N(CH₃)C(O)O, C(O)ONH, or C(O)ON(CH₃).

In some embodiments, the cationic lipid is selected from a cationic lipid of formula (Ib):

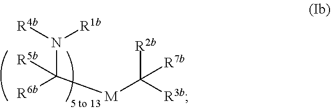

R^{1b} is selected from the group consisting of C₅₋₃₀ alkyl, C₅₋₂₀ alkenyl, —R^{b}*Y^{c}R^{b}″, —Y^{b}R^{b}″, and —R^{b}″M^{b}′R^{b}′;
R^{2b} and R^{3b} are independently selected from the group consisting of H, C₁₋₁₄ alkyl, C₂₋₁₄ alkenyl, —R^{b}*Y^{b}R^{b}″, —Y^{b}R^{b}″, and —R^{b}*OR^{b}″, or R^{2b} and R^{3b} together with the atoms to which they are attached form heterocyclyl or carbocyclyl;
R^{4b} is selected from the group consisting of C₃₋₆ carbocyclyl, —(CH₂)₁₋₅Q, —(CH₂)₁₋₅CHQ^{b}R^{b}, —CHQ^{b}R^{b}, —CQ^{b}(R^{b})₂, and unsubstituted C₁₋₆ alkyl, wherein Q^{b} is selected from carbocyclyl, heterocyclyl, —OR^{b}, —O(CH₂)₁₋₅N(R^{b})₂, —C(O)OR^{b}, —OC(O)R^{b}, —CX₃, —CX₂H, —CXH₂, —CN, —N(R^{b})₂, —C(O)N(R^{b})₂, —N(R^{b})C(O)R^{b}, —N(R^{b})S(O)₂R^{b}, —N(R^{b})C(O)N(R^{b})₂, —N(R^{b})C(S)N(R^{b})₂, —N(R^{b})R^{8b}, —O(CH₂)₁₋₅OR^{b}, —N(R^{b})C(═NR^{9b})N(R^{b})₂, —N(R^{b})C(═CHR^{9b})N(R^{b})₂, —OC(O)N(R^{b})₂, —N(R^{b})C(O)OR^{b}, —N(OR^{b})C(O)R^{b}, —N(OR^{b}) S(O)₂R^{b}, —N(OR^{b})C(O)OR^{b}, —N(OR^{b})C(O)N(R^{b})₂, —N(OR^{b})C(S)N(R^{b})₂, —N(OR^{b})C(═NR^{9b})N(R^{b})₂, —N(OR^{b})C(═CHR^{9b})N(R^{b})₂, —C(═NR^{9b})N(R^{b})₂, —C(═NR^{9b})R^{b}, —C(O)N(R^{b}) OR^{b}, and —C(R^{b})N(R^{b})₂C(O)OR^{b}; each R^{5b} is independently selected from the group consisting of C₁₋₃ alkyl, C₂₋₃ alkenyl, and H; each R^{6b} is independently selected from the group consisting of C₁₋₃ alkyl, C₂₋₃ alkenyl, and H; M and M^{b}′ are independently selected from —C(O)O—, —OC(O)—, —C(O)N(R^{b}′)—, —N(R^{b}′)C(O)—, —C(O)—, —C(S)—, —C(S)S—, —SC(S)—, —CH(OH)—, —P(O)(OR^{b}′)O—, —S(O)₂—, —S—S—, aryl, and heteroaryl; R^{7b} is selected from the group consisting of C₁₋₃ alkyl, C₂₋₃ alkenyl, and H; R^{8b} is selected from the group consisting of C₃₋₆ carbocyclyl and heterocyclyl; R^{9b} is selected from the group consisting of H, CN, NO₂, C₁₋₆ alkyl, —OR^{b}, —S(O)₂R^{b}, —S(O)₂N(R^{b})₂, C₂₋₆ alkenyl, C₃₋₆ carbocyclyl and heterocyclyl; each R^{b} is independently selected from the group consisting of C₁₋₃ alkyl, C₂₋₃ alkenyl, and H; each R^{b}′ is independently selected from the group consisting of C₁₋₁₈ alkyl, C₂₋₁₈ alkenyl, —R^{b}*YR^{b}″, —YR^{b}″, and H; each R^{b}″ is independently selected from the group consisting of C₃₋₁₄ alkyl and C₃₋₁₄ alkenyl; each R^{b}* is independently selected from the group consisting of C₁₋₁₂ alkyl and C₂₋₁₂ alkenyl; each Y^{b} is independently C₃₋₆ carbocyclyl; and each X^{b} is independently selected from the group consisting of F, Cl, Br, and I.

In some embodiments, the cationic lipid is selected from a cationic lipid of formula (Ic):

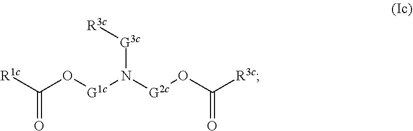

wherein G^{1c} and G^{2c} are each independently unsubstituted —(CH₂)₆—, —(CH₂)₇—, —(CH₂)₈—, —(CH₂)₉—, or —(CH₂)₁₀—;
G^{3c} is unsubstituted —(CH₂)—, —(CH₂)₂—, —(CH₂)₃—, —(CH₂)₄—, —(CH₂)₅—, —(CH₂)₆—, —(CH₂)₇—, —(CH₂)₈—, —(CH₂)₉—, (CH₂)₁₀—, —(CH₂)₁₁—, or —(CH₂)₁₂—;
R^{1c} and R^{2c} are each independently C₆₋₂₄ alkyl or C₆₋₂₄ alkenyl;
R^{3c} is OR^{5c}, CN, —C(═O)OR⁴, —OC(═O)R⁴, or —NR^{5c}C(═O)R^{4c}, R^{4c} is C₁₋₁₂ hydrocarbonyl; and
R^{5c} is H, methyl, ethyl, n-propyl, isopropyl, n-butyl, isobutyl, sec-butyl, or tert-butyl.

In some examples, the cationic lipid is selected from a cationic lipid of formula (Ida) or (Idb):

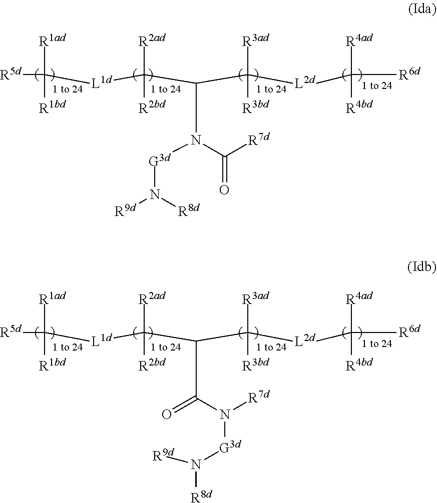

L^{1d} and L^{2d} are each independently —O(C═O)—, —(C═O)O—, —C(═O)—, —O—, —NR^{ad}C(═O)—, —C(═O)NR^{ad}—, —NR^{ad}C(═O)NR^{ad}—, —OC(═O)NR^{ad}—, —NR^{ad}C(═O)O—, or a bond;
G^{3d} is —(CH₂)—, —(CH₂)₂—, —(CH₂)₃—, —(CH₂)₄—, —(CH₂)₅—, or —(CH₂)₆—; R^{ad} is H or C₁₋₁₂ hydrocarbonyl;
R^{1ad} and R^{1bd} are independently at each occurrence (a) H or C₁₋₁₂ hydrocarbonyl; or (b) R^{1ad} is H or C₁₋₁₂ hydrocarbonyl, and R^{1bd}, together with the carbon atom to which it is attached, and the adjacent R^{1bd}, together with the carbon atom to which it is attached, form a carbon-carbon double bond;
R^{2ad} and R^{2bd} are independently at each occurrence (a) H or C₁₋₁₂ hydrocarbonyl; or (b) R^{2ad} is H or C₁₋₁₂ hydrocarbonyl, and R^{2bd}, together with the carbon atom to which it is attached, and the adjacent R^{2bd}, together with the carbon atom to which it is attached, form a carbon-carbon double bond;
R^{3ad} and R^{3bd} are independently at each occurrence (a) H or C₁₋₁₂ hydrocarbonyl; or (b) R^{3ad} is H or C₁₋₁₂ hydrocarbonyl, and R^{3bd}, together with the carbon atom to which it is attached, and the adjacent R^{3bd}, together with the carbon atom to which it is attached, form a carbon-carbon double bond;
R^{4ad} and R^{4bd} are independently at each occurrence (a) H or C₁₋₁₂ hydrocarbonyl; or (b) R^{4ad} is H or C₁₋₁₂ hydrocarbonyl, and R^{4bd}, together with the carbon atom to which it is attached, and the adjacent R^{4bd}, together with the carbon atom to which it is attached, form a carbon-carbon double bond;
R^{5d} and R^{6d} are each independently H or methyl;
R^{7d} is C₆₋₁₆ hydrocarbonyl optionally substituted with —(C═O)OR^{bd}, —O(C═O)R^{bd}, —C(═O)R^{bd}, —OR^{bd}, —C(═O) SR^{bd}, —NR^{ad}R^{bd}, —NR^{ad}C(═O)R^{bd}, —C(═O)NR^{ad}R^{bd}, —NR^{ad}C(═O)NR^{ad}R^{bd}, —OC(═O)NR^{ad}R^{bd}, or —NR^{ad}C(═O)OR^{bd}, wherein R^{ad} is H or C₁₋₁₂ hydrocarbonyl; R^{bd} is C₁₋₁₅ hydrocarbonyl;
R^{8d} and R^{9d} are each independently C₁₋₁₂ hydrocarbonyl; or R^{8d} and R^{9d}, together with the nitrogen atom to which they are attached, form a 5-, 6-, or 7-membered heterocyclic ring.

The term “DLin-MC3-DMA” refers to a substance with CAS No. 1224606-06-7. The structure of the substance is

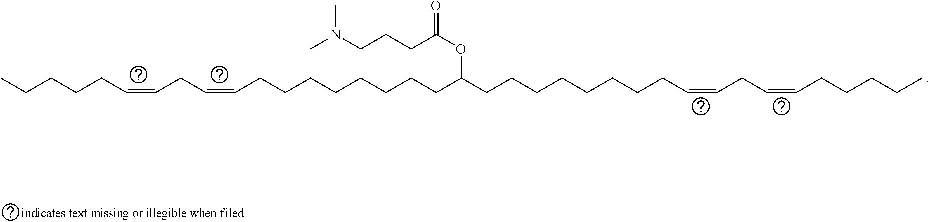 [SMILES: CCCCC/C=C\C/C=C\CCCCCCCCC(CCCCCCCC/C=C\C/C=C\CCCCC)OC(=O)CCCN(C)C]

The term “DSDMA” refers to a substance with CAS No. 871258-14-9, and the structure of the substance is

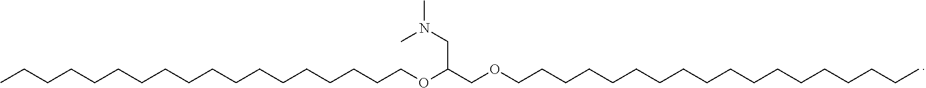 [SMILES: CCCCCCCCCCCCCCCCCCOCC(CN(C)C)OCCCCCCCCCCCCCCCCCC]

The term “DLenDMA” refers to a substance with CAS No. 874291-25-5, and the structure of the substance is

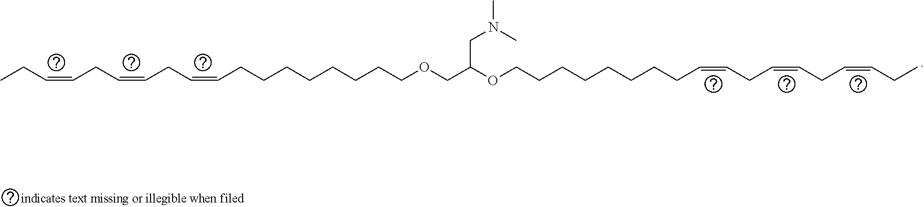 [SMILES: CC/C=C\C/C=C\C/C=C\CCCCCCCCOCC(CN(C)C)OCCCCCCCC/C=C\C/C=C\C/C=C\CC]

The term “DODMA” refers to a substance with CAS No. 104162-47-2, and the structure of the substance is

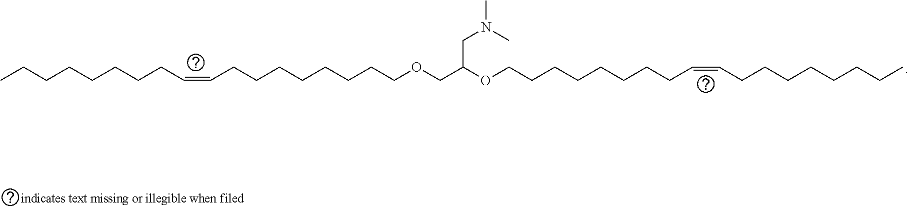 [SMILES: CCCCCCCC/C=C\CCCCCCCCOCC(CN(C)C)OCCCCCCCC/C=C\CCCCCCCC]

The term “A6” refers to a substance with the structure

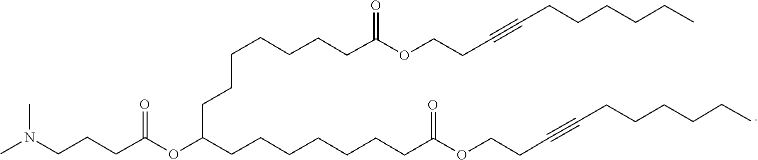 [SMILES: CCCCCCC#CCCOC(=O)CCCCCCCC(CCCCCCCC(=O)OCCC#CCCCCCC)OC(=O)CCCN(C)C]

The term “OF-02” refers to a substance with the structure

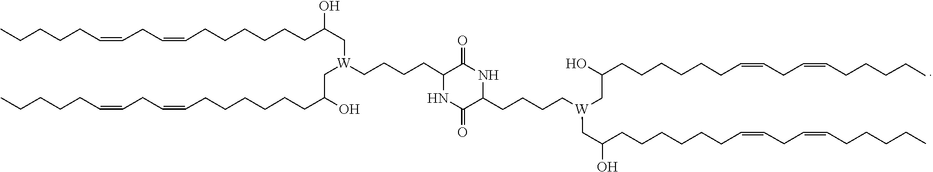

The term “A18-Iso5-2DC18” refers to a substance with the structure

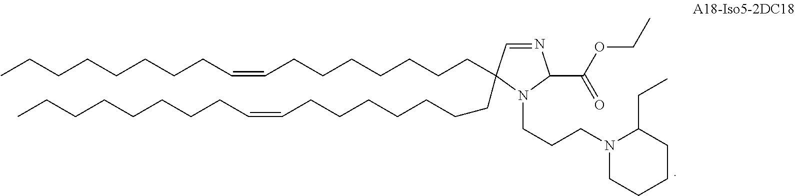 [SMILES: CCCCCCCC/C=C\CCCCCCCC1(CCCCCCC/C=C\CCCCCCCC)C=NC(C(=O)OCC)N1CCCN1CCCCC1CC]

The term “98N₁₂-5” refers to a substance with the structure

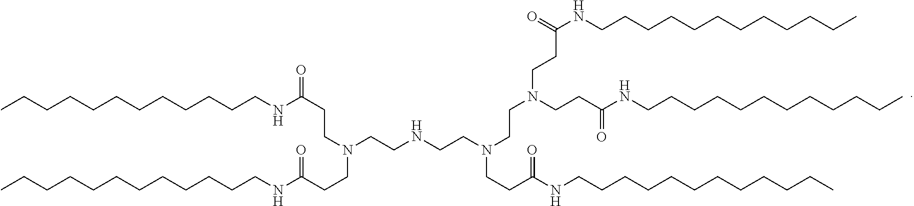 [SMILES: CCCCCCCCCCCCNC(=O)CCN(CCNCCN(CCC(=O)NCCCCCCCCCCCC)CCN(CCC(=O)NCCCCCCCCCCCC)CCC(=O)NCCCCCCCCCCCC)CCC(=O)NCCCCCCCCCCCC]

The term “9A1P9” refers to a substance with the structure

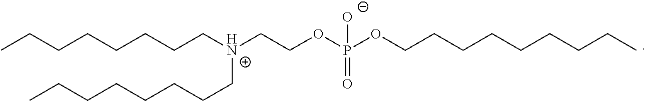

The term “C12-200” refers to a substance with the structure

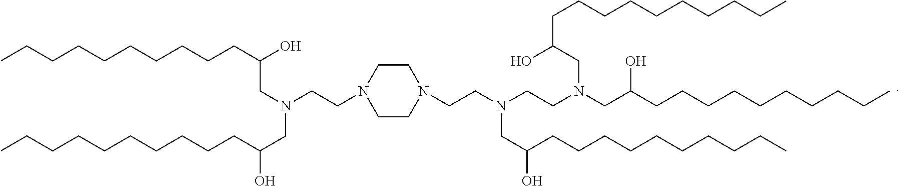 [SMILES: CCCCCCCCCCC(O)CN(CCN1CCN(CCN(CC(O)CCCCCCCCCC)CC(O)CCCCCCCCCC)CC1)CCN(CC(O)CCCCCCCCCC)CC(O)CCCCCCCCCC]

The term “cKK-E12” refers to a substance with the structure

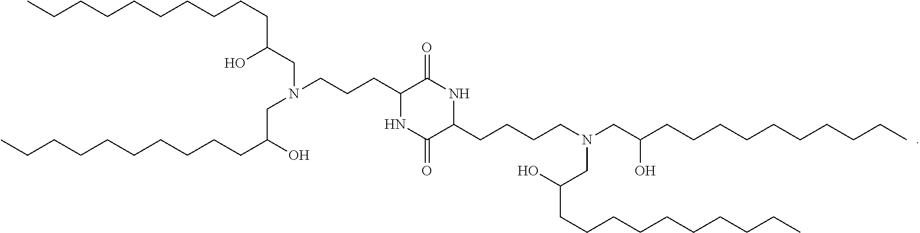 [SMILES: CCCCCCCCCCC(O)CN(CCCCC1NC(=O)C(CCCN(CC(O)CCCCCCCCCC)CC(O)CCCCCCCCCC)NC1=O)CC(O)CCCCCCCCCC]

The term “G0-C14” refers to a substance with the structure

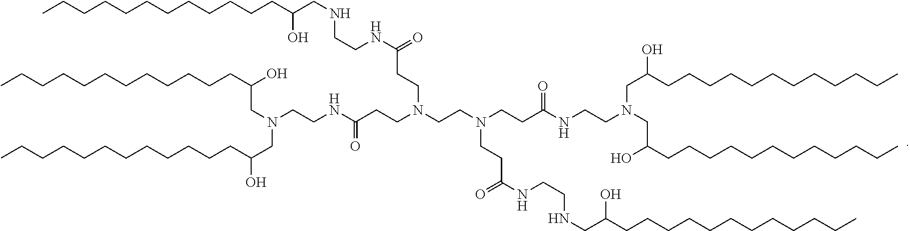 [SMILES: CCCCCCCCCCCCC(O)CNCCNC(=O)CCN(CCC(=O)NCCN(CC(O)CCCCCCCCCCCC)CC(O)CCCCCCCCCCCC)CCN(CCC(=O)NCCNCC(O)CCCCCCCCCCCC)CCC(=O)NCCN(CC(O)CCCCCCCCCCCC)CC(O)CCCCCCCCCCCC]

The term “L319” refers to a substance with the structure

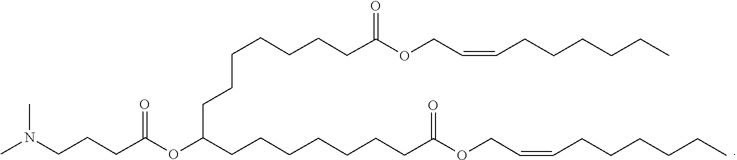 [SMILES: CCCCCC/C=C\COC(=O)CCCCCCCC(CCCCCCCC(=O)OC/C=C\CCCCCC)OC(=O)CCCN(C)C]

The term “SM-102” refers to a substance with CAS No. 2089251-47-6, the structure of which is

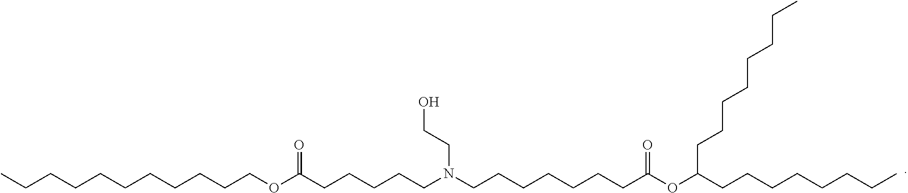 [SMILES: CCCCCCCCCCCOC(=O)CCCCCN(CCO)CCCCCCCC(=O)OC(CCCCCCCC)CCCCCCCC]

The term “ALC-0315” refers to a substance with CAS No. 2036272-55-4, the structure of which is

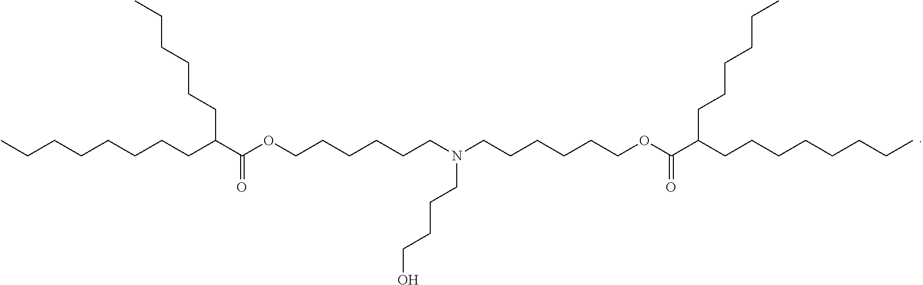 [SMILES: CCCCCCCCC(CCCCCC)C(=O)OCCCCCCN(CCCCO)CCCCCCOC(=O)C(CCCCCC)CCCCCCCC]

The term “A9” refers to a substance with the structure

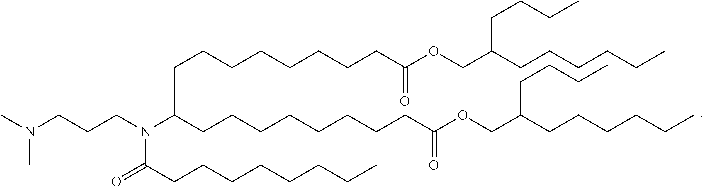 [SMILES: CCCCCCCCC(=O)N(CCCN(C)C)C(CCCCCCCCC(=O)OCC(CCCC)CCCCCC)CCCCCCCCC(=O)OCC(CCCC)CCCCCC]

The term “Lipid 2,2(8,8)4CCH3” refers to a substance with the structure

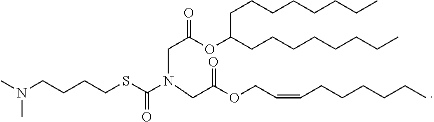 [SMILES: CCCCCC/C=C\COC(=O)CN(CC(=O)OC(CCCCCCCC)CCCCCCCC)C(=O)SCCCCN(C)C]

The term “CL1” refers to a substance with the structure

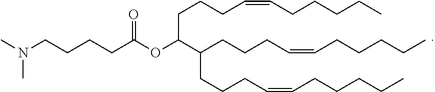 [SMILES: CCCCC/C=C\CCCC(CCC/C=C\CCCCC)C(CCC/C=C\CCCCC)OC(=O)CCCCN(C)C]

The term “LP01” refers to a substance with the structure

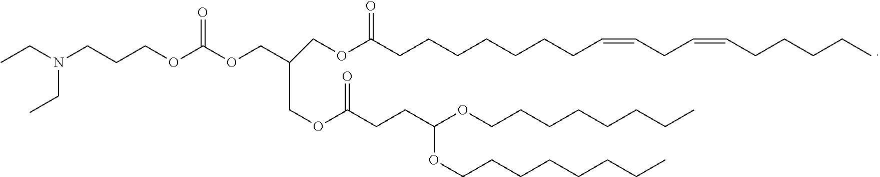 [SMILES: CCCCC/C=C\C/C=C\CCCCCCCC(=O)OCC(COC(=O)CCC(OCCCCCCCC)OCCCCCCCC)COC(=O)OCCCN(CC)CC]

The term “MC3” or “DLin-MC3-DMA” refers to a substance with the structure

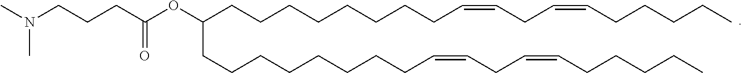 [SMILES: CCCCC/C=C\C/C=C\CCCCCCCCC(CCCCCCCC/C=C\C/C=C\CCCCC)OC(=O)CCCN(C)C]

The term “PEG2000-DMG” refers to 1,2-dimyristoyl-rac-glycero-3-methoxypolyethylene glycol-2000, with the Chinese name being dimyristoylglycerol-polyethylene glycol 2000. It has a molecular formula of C₁₂₂H₂₄₂O₅₀, a molecular weight of 2509.2, and a structural formula of

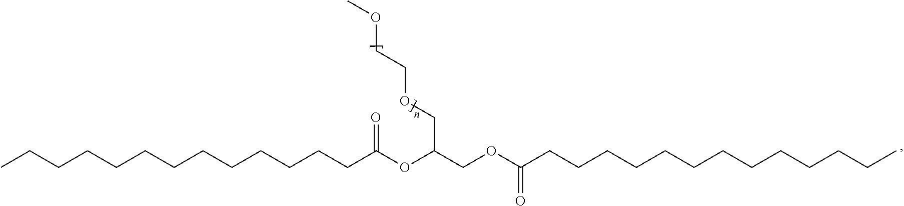 [SMILES: CCCCCCCCCCCCCC(=O)OCC(COCCOC)OC(=O)CCCCCCCCCCCCC]

with n=45.

In addition to the cationic lipids explicitly described above, other cationic lipids with a net positive charge at approximately the physiological pH can be included in the lipid particles of the present invention. Such cationic lipids include, but are not limited to, N,N-dioleyl-N,N-dimethylammonium chloride (“DODAC”); N-(2,3-dioleyloxy) propyl-N,N—N-triethylammonium chloride (“DOTMA”); N,N-distearoyl-N,N-diethylammonium bromide (“DDAB”); N-(2,3-dioleoyloxy) propyl)-N,N,N-triethylammonium chloride (“DOTAP”); 1,2-dioleyloxy-3-trimethylaminopropane hydrochloride (“DOTAP.Cl”); 3-(N—(N′,N′-dimethylaminoethane)-carbamoyl) cholesterol (“DC-Chol”), N-(1-(2,3-dioleyloxy) propyl)-N-2-(sperminecarboxamido)ethyl)-N,N-dimethylammonium trifluoroacetate (“DOSPA”), di-octadecyl-amido-glycyl-spermine (“DOGS”), 1,2-dioleyl-sn-3-phosphorylethanolamine (“DOPE”), 1,2-dioleoyl-3-dimethylpropylamine (“DODAP”), N,N-dimethyl-2,3-dioleyloxy) propylamine (“DODMA”), and N-(1,2-dimyristyloxyprop-3-yl)-N,N-dimethyl-N-hydroxyethylammonium bromide (“DMRIE”). In addition, many commercial formulations of cationic lipids can be used, such as, for example, LIPOFECTIN (including DOTMA and DOPE, available from GIBCO/BRL) and LIPOFECTAMINE (including DOSPA and DOPE, available from GIBCO/BRL). In a particular embodiment, the cationic lipid is an amino lipid.

The abbreviation “Chol” refers to cholesterol.

The ratio of “N/P” refers to the number of moles of the positive charges of all cationic lipids contained in the composition: the number of moles of phosphate radicals.

The term “CaLNP” refers to the calcium-containing cationic lipid nanoparticles according to any of the preceding aspects.

The term “mammal” refers to any mammal species such as human, mouse, rat, dog, cat, hamster, guinea pig, rabbit, and livestock.

### BRIEF DESCRIPTION OF THE DRAWINGS

FIG. 1 shows an investigation on CaLNP formulations with different calcium acetate concentrations for gene knock-down efficiency by siRNA.

FIG. 2 shows an investigation on CaLNP formulations prepared by different ways of adding calcium acetate for gene knock-down efficiency by siRNA.

FIG. 3 shows that CaLNPs prepared from different ionizable cationic materials have increased gene knock-down efficiency by siRNA as compared with LNPs.

FIG. 4 shows that CaLNPs prepared from different neutral lipid materials have enhanced gene knock-down efficiency by siRNA as compared with LNPs.

FIG. 5 shows a comparison in siRNA transfection efficiency between LNPs mixed with calcium acetate or EPC-liposome-encapsulated calcium acetate and CaLNPs.

FIG. 6 shows a comparison in mRNA transfection efficiency.

FIG. 7 shows a comparison in mRNA transfection efficiency.

FIG. 8 shows a comparison in DNA transfection efficiency.

FIG. 9 shows a comparison of living imaging of mice administered with CaLNPs encapsulating pGL3-control plasmid (left, Example 3-1, with a DNA dose of 0.1 mg/kg) and those administered with LNPs (right, Example 8-1, with a DNA dose of 0.1 mg/kg).

FIG. 10 shows a comparison of living imaging of mice administered with LNPs encapsulating Fluc-mRNA (left, Example 7-1, with an mRNA dose of 0.3 mg/kg; right, Example 7-1, with an mRNA dose of 0.1 mg/kg) and CaLNPs (middle, Example 4-2, with an mRNA dose of 0.06 mg/kg).

FIG. 11 shows the in vitro silencing efficiency of CaLNPs with different Ca²⁺ concentrations in Experimental Example 8.

FIG. 12 shows the in vitro silencing efficiency of different cationic lipid CaLNPs and LNPs in Experimental Example 8.

FIG. 13 shows the FVII gene silencing efficiency of the formulations of Experimental Example 9 when administered intravenously to mice.

FIG. 14 shows the TTR gene silencing efficiency of the formulations of Experimental Example 9 when administered intravenously to mice.

### DETAILED DESCRIPTION OF EMBODIMENTS

Specific embodiments of the present invention will be described in detail below. It should be noted that the embodiments described here are for illustration only and are not used to limit the present invention. In the following description, in order to provide a thorough understanding of the present invention, numerous specific details are set forth; however, it would be obvious to those skilled in the art that these specific details are not necessary to practice the present invention.

The detection methods used in the present invention are all conventional or general detection methods in the art. Where a detection method involves the use of a kit, it shall be operated according to the instructions in the commercial kit. If it is necessary to use instruments and apparatuses for detection, a usual operation method is to observe a conventional operation method in the art for operation and detection. Unless otherwise specified, the following detection methods are carried out according to the description of the present invention.

List of abbreviations

| | |
|---|---|
| DODMA | N,N-dimethyl-(2,3-dioleyloxy)propylamine |
| DOPE | 1,2-Dioley1-sn-3-phosphorylethanolamine |
| DSPC | Distearoylphosphatidylcholine |
| PEG-DMG | Polyethylene glycol-dimyristoylglycerol |
| siRNA | Small interfering RNA |
| GAPDH | Glyceraldehyde-3-phosphate dehydrogenase |
| Tris | Tris(hydroxymethyl)aminoethane |
| Triton | Triton |
| std | Standard |
| Fluc | Firefly Luciferase |

(1) Determination Method for siRNA Content:

Method I: Qubit microRNA detection kit is used with Qubit3 Fluorometer to quantify microRNA. Initially, a sample is diluted 20 times with 1% Triton-100, and then, the diluted sample is detected with Qubit microRNA detection kit on Qubit3 Fluorometer.

Method II: RiboGreen is an ultra-sensitive fluorescent nucleic acid dye for the quantitative detection of the content of RNA in a solution. RiboGreen kit is used for detection on a fluorescence microplate reader. The sample is diluted with 0.5% Triton-100 to a suitable concentration for detection.

(2) Determination Method for mRNA Content:

Method I: The sample is diluted 20 times with 1% Triton-100, and mRNA is then quantified by Qubit3 Fluorometer according to the instructions of Qubit mRNA HS detection kit.

Method II: RiboGreen is an ultra-sensitive fluorescent nucleic acid dye for the quantitative detection of the content of RNA in a solution. RiboGreen kit is used for detection on a fluorescence microplate reader. The sample is diluted with 0.5% Triton-100 to a suitable concentration for detection.

### (3) Determination Method for DNA Content:

The sample is diluted 20 times with 1% Triton-100, and DNA is then quantified by Qubit3 Fluorometer according to the instructions of Qubit DNA HS detection kit.

### (4) Determination Method for Hydrate Particle Size:

The average particle sizes of the CaLNPs prepared by the present invention are all tested by Malvern particle size analyzer, model Nano-ZS. During operation, the solution to be tested is diluted 10 times with a Tris-containing buffer with pH 7.4 or water for determination.

### (5) Determination Method for Nucleic Acid Content and Encapsulation Efficiency

Encapsulation efficiency is a very critical indicator for CaLNP-RNA and represents the ratio of RNA encapsulated in lipid nanoparticles to the total RNA. Initially, CaLNP-RNA is directly mixed with a fluorescent dye working solution to detect the amount of RNA free from CaLNPs in the CaLNP-RNA solution; next, Triton-100 is used to destroy the structure of the CaLNPs so that RNA is released into the external solution, which is then mixed with a fluorescent dye working solution to detect the total amount of RNA in the solution. The difference between the two is the amount of RNA encapsulated in LNP particles, so as to derive the encapsulation efficiency by calculation. The method is the same as the content detection method.

Specifically, Qubit™ microRNA quantitative kit (Thermofisher) is used for detection.

### Nucleic Acid Content:

Initially, a formulation is diluted by an appropriate multiple with 1% Triton-100, and then, the diluted formulation is detected with Qubit microRNA detection kit on Qubit3 Fluorometer.

Encapsulation efficiency: Encapsulation efficiency is a very critical indicator for CaLNP-RNA and represents the ratio of RNA encapsulated in lipid nanoparticles to the total RNA. Initially, CaLNP-RNA is directly mixed with a fluorescent dye working solution, and the amount of RNA free from the CaLNPs in the CaLNP-RNA solution is detected. Next, Triton-100 is used to destroy the structure of the CaLNPs so that RNA is released into the external solution, which is then mixed with a fluorescent dye working solution to detect the total amount of RNA in the solution. The difference between the two is the amount of RNA encapsulated in LNP particles, so as to derive the encapsulation efficiency by calculation.

(6) Detection methods for cholesterol and DSPC contents in different formulations Determination is carried out according to high performance liquid chromatography (Chinese Pharmacopoeia, 2020 Edition, Volume IV, General Rule 0512).

Chromatographic conditions:

| | |
|---|---|
| Instrument | High Performance Liquid Chromatograph (Agilent) |
| Detector | Differential detector |
| Chromatographic | Chromatographic column with octadecylsilane-bonded silica |
| column | gel as a filler or an equivalent chromatographic column |
| Mobile phase | Methanol - tetrahydrofuran - 0.17 mol/L ammonium acetate |
| | solution (94:5:1) |
| Column temperature | 35° C. |
| Injection volume | 20 μl |
| Diluent | Methanol:chloroform (3:1) |

.

### (1) Preparation of Mobile Phase

About 0.65518 g of ammonium acetate is weighed, 50 mL of water is added, the materials are mixed until uniform and suction-filtered to prepare a 0.17 mol/L ammonium acetate solution; and a mobile phase is prepared according to a ratio, mixed well, and ultrasonicated for 10 minutes.

### (2) Preparation of Control Solution

19.36 mg of cholesterol and 12.36 mg of DSPC are weighed, put into a 10 mL volumetric flask, dissolved with an appropriate amount of a diluent, made up to the marked volume, and mixed until uniform, denoted as std-1. std-1 is then diluted 100 times as a control solution.

### (3) Preparation of Test Solution

0.1 mL of the formulation is taken, 0.6 ml of a diluent is added, and the materials are mixed until uniform, as a test sample. It is introduced into a liquid phase for detection.

### (4) Detection Method for Cholesterol Concentration

Determination is carried out according to a free cholesterol (FC) content detection kit (Beijing Boxbio, article number: AKFA001C).

Blank solution: anhydrous ethanol

Control solution: Cholesterol is taken and dissolved to 1 μmol/ml with anhydrous ethanol.

Test solution: 100 μl of the formulation is taken, 20 μl of 1% Triton is added, and the materials are mixed until uniform.

Determination method: 50 μl of the blank solution, 50 μl of the control solution, and 50 μl of the test solution are respectively taken, and 950 μl of an FC working solution (from a detection kit) is added and mixed until uniform; after 15 minutes of color development at 37° C., the mixture is put into an ultraviolet-visible spectrophotometer for detection, with the detection wavelength being 500 nm and the optical path being 1 cm. The absorbance values are recorded as A_{blank}, A_{control}, and Aₜₑₛₜ, respectively.

$${C}_{test} = 1.2 * ( {A}_{test} - {A}_{blank} ) / ( {A}_{control} - {A}_{blank} ) * {C}_{control}$$

### (5) Detection Method for Ca²⁺ Concentration

Determination is carried out according to a calcium ion (Ca) determination kit (arsenazo III method) (Beijing Leadman Biochemistry Co., Ltd.).

Blank solution: water

Control solution: 100 μl of a Ca²⁺ ion standard solution (2.46 mmol/L) is taken from a kit, 900 μl of water is added, and these materials are mixed until uniform.

A free Ca²⁺ sample solution is a formulation sample which is directly taken.

Total Ca²⁺ test solution: 100 μl of the formulation sample is taken, and 20 μl of 1% Triton is added.

Determination method: The blank solution, the control solution, the free Ca²⁺ test solution, and the total Ca²⁺ test solution (100 μl) are respectively taken, 900 μl of a calcium ion (Ca) determination reagent is added and mixed until uniform, and the mixture is put into an ultraviolet-visible spectrophotometer for detection, with the detection wavelength being 600 nm and the optical path being 1 cm. The absorbance values are recorded as A_{blank}, A_{control}, A_{free Ca test}, and A_{total Ca test}, respectively. AC represents the concentration of calcium ions in lipid nanoparticles relative to the total volume of the test sample.

$$\begin{matrix}{C}_{free Ca test} = ( {A}_{free Ca test} - {A}_{blank} ) / ( {A}_{control} - {A}_{blank} ) * {C}_{control} \\ {C}_{total Ca test} = 1.2 * ( {A}_{total Ca test} - {A}_{blank} ) / ( {A}_{control} - {A}_{blank} ) * {C}_{control} \\ Δ C = {C}_{total ca test} - {C}_{free Ca test}\end{matrix}$$

### Example 1. Preparation of siRNA-CaLNP Formulations with Different Concentrations of Calcium Acetate

65.0 mg of DLin-MC3-DMA, 31.0 mg of cholesterol, 16.5 mg of DSPC, and 8.0 mg of PEG2000-DMG were weighed and dissolved by adding 10 ml of ethanol to form an organic phase; 1 mg of siRNA (targeting human GAPDH mRNA, Seq. No. 1) was taken and mixed with 0.9 ml of water until uniform to form an siRNA mother liquid; and 90 μl of the siRNA mother liquid was taken and diluted with 810 μl of calcium acetate solutions with different concentrations to form aqueous phases, wherein the pH value of the calcium acetate solutions with different concentrations was adjusted to 5 with acetic acid; and 0.9 ml of the aqueous phase and 0.2 ml of the organic phase were taken and mixed manually until uniform to obtain intermediate I, which was dialyzed in a Tris-containing buffer with pH 7.4 by a dialysis bag with a molecular weight cut-off of 8000D. The internal phase dialysate was collected, filtered, and sterilized to finally obtain siRNA-CaLNPs. The different concentrations of the calcium acetate solutions in Table 1 were 25 mmol/1, 50 mmol/1, 100 mmol/1, 200 mmol/l, and 400 mmol/l.

Seq. No. 1:
Sense:
GUAUG ACAAC AGCCU CAAGT T
Anti-sense:
CUUGA GGCUG UUGUC AUACT T

TABLE 1
Scheme of dosing siRNA-LNP with different calcium acetate concentrations

| formulation code | 1-1 | | 1-2 | | 1-3 | | 1-4 | | 1-5 | |
|---|---|---|---|---|---|---|---|---|---|---|
| Concentration of calcium acetate | 25 | mmol/l | 50 | mmol/l | 100 | mmol/l | 200 | mmol/l | 400 | mmol/l |
| Dosage of calcium acetate | 0.81 | mL | 0.81 | mL | 0.81 | mL | 0.81 | mL | 0.81 | mL |
| Dosage of siRNA mother liquid | 0.09 | mL | 0.09 | mL | 0.09 | mL | 0.09 | mL | 0.09 | mL |
| Dosage of organic phase | 0.2 | mL | 0.2 | mL | 0.2 | mL | 0.2 | mL | 0.2 | mL |
| 0.2 mol/l Tris buffer | 0.073 | mL | 0.146 | mL | 0.293 | mL | 0.585 | mL | 1.17 | mL |

TABLE 2
Determination data of each formulation in Example 1

| formulation code | 1-1 | 1-2 | 1-3 | 1-4 | 1-5 |
|---|---|---|---|---|---|
| Dosage concentration of | 25 mmol/l | 50 mmol/l | 100 mmol/l | 200 mmol/l | 400 mmol/l |
| calcium acetate | | | | | |
| siRNA content (μg/mL) | 35 | 36 | 36 | 29 | 14 |
| Encapsulation efficiency | 96.47 | 95.90 | 96.74 | 93.38 | 89.92 |
| (%) | | | | | |
| Average particle size | 134.2 | 112.0 | 98.15 | 130.4 | 149.2 |
| (nm) | | | | | |

TABLE 3
Detection data of cholesterol and DSPC in each formulation in Example 1

| formulation code | Cholesterol concentration, μg/ml | DSPC concentration, μg/ml |
|---|---|---|
| 1-1 | 239.13 | 130.68 |
| 1-2 | 219.44 | 110.25 |
| 1-3 | 232.99 | 125.10 |
| 1-5 | 116.21 | 58.25 |

### Example 2. Preparation of 200 Mmol/l Calcium Acetate mRNA-CaLNP Formulation

65.0 mg of DLin-MC3-DMA, 31.0 mg of cholesterol, 16.5 mg of DSPC, and 8.0 mg of PEG2000-DMG were weighed and dissolved by adding 10 ml of ethanol to form an organic phase; 1 mg of Firefly Luciferase mRNA (N¹-Me-Pseudo UTP) was taken, added to 8 ml of a 200 mmol/l calcium acetate solution with pH 5, and mixed until uniform to form an aqueous phase; and the organic phase at 4 ml/min and the aqueous phase at 18 ml/min were mixed in a Y-tube to collect intermediate I.

Intermediate I was adjusted to pH 7 and dialyzed in an ice bath with a Tris-containing buffer with pH 7.4 using a dialysis bag with a molecular weight cut-off of 8000D. The internal liquid was collected, filtered, and sterilized to obtain mRNA-CaLNP, denoted as 2-1. After measurement, the content was 39 μg/ml, the encapsulation efficiency was 95.12%, and the average particle size was 79.0 nm.

After ultrafiltration and concentration, the content was 400 μg/ml.

Firefly Luciferase mRNA (N¹-Me-Pseudo UTP) was a commercial mRNA (product number: DD4511-01) commercially available from Nanjing Vazyme. After entering cells, Firefly Luciferase mRNA (N¹-Me-Pseudo UTP) expressed firefly luciferase protein, which catalyzed the oxidation of D-fluorescein in an ATP-dependent manner, resulting in fluorescence at 560 nm wavelength.

### Example 3. Preparation of 200 Mmol/l Calcium Acetate DNA-CaLNP Formulation

65.0 mg of DLin-MC3-DMA, 31.0 mg of cholesterol, 16.5 mg of DSPC, and 8.0 mg of PEG2000-DMG were weighed and dissolved by adding 10 ml of ethanol to form an organic phase;

0.5 mg/0.5 ml of pGL3-control plasmid was taken, added to 4 ml of a 200 mmol/l calcium acetate solution with pH 5, and mixed until uniform to form an aqueous phase; and the organic phase at 4 ml/min and the aqueous phase at 18 ml/min were mixed in a Y-tube to collect an intermediate.

The intermediate at 20 ml/min and a 0.2 M Tris buffer with pH 8.5 at 10.5 ml/min were mixed in a Y-tube to collect a product, which was dialyzed in a Tris-containing buffer with pH 7.4 by a dialysis bag with a molecular weight cut-off of 8000D. The internal liquid was collected, filtered, and sterilized to obtain pGL3-control-CaLNP, denoted as 3-1. After measurement, the content was 35 μg/ml, the encapsulation efficiency was 91.01%, and the average particle size was 125.9 nm.

0.5 mg/0.5 ml of EGFP plasmid was taken, added to 4 ml of a 200 mmol/l calcium acetate solution with pH 5, and mixed until uniform to form an aqueous phase; and the organic phase at 4 ml/min and the aqueous phase at 18 ml/min were mixed in a T-tube to collect an intermediate. The intermediate was adjusted to pH 7 and dialyzed with a Tris buffer with pH 7.4 using a dialysis bag with a molecular weight cut-off of 8000D. The internal liquid was collected, filtered, and sterilized to obtain EGFP-CaLNPs, denoted as 3-2. After measurement, the content was 31 μg/ml, the encapsulation efficiency was 95.70%, and the average particle size was 117.4 nm.

After ultrafiltration and concentration, the content was 1 mg/ml.

The pGL3-control plasmid was a plasmid commercially available from Promega.

The sequence thereof was:

Seq No 2:
ggtaccgagctcttacgcgtgctagcccgggctcgagatctgcgatctgcatctcaattagtcagcaaccatagtcccgcccctaactccgcccatcccgcccct
aactccgcccagttccgcccattctccgccccatcgctgactaattttttttatttatgcagaggccgaggccgcctcggcctctgagctattccagaagtagtgag
gaggcttttttggaggcctaggcttttgcaaaaagcttggcattccggtactgttggtaaagccaccatggaagacgccaaaaacataaagaaaggcccggcgc
cattctatccgctggaagatggaaccgctggagagcaactgcataaggctatgaagagatacgccctggttcctggaacaattgcttttacagatgcacatatcg
aggtggacatcacttacgctgagtacttcgaaatgtccgttcggttggcagaagctatgaaacgatatgggctgaatacaaatcacagaatcgtcgtatgcagtg
aaaactctcttcaattctttatgccggtgttgggcgcgttatttatcggagttgcagttgcgcccgcgaacgacatttataatgaacgtgaattgctcaacagtatggg
catttcgcagcctaccgtggtgttcgtttccaaaaaggggttgcaaaaaattttgaacgtgcaaaaaaagctcccaatcatccaaaaaattattatcatggattctaa
aacggattaccagggatttcagtcgatgtacacgttcgtcacatctcatctacctcccggttttaatgaatacgattttgtgccagagtccttcgatagggacaagac
aattgcactgatcatgaactcctctggatctactggtctgcctaaaggtgtcgctctgcctcatagaactgcctgcgtgagattctcgcatgccagagatcctattttt
ggcaatcaaatcattccggatactgcgattttaagtgttgttccattccatcacggttttggaatgtttactacactcggatatttgatatgtggatttcgagtcgtcttaa
tgtatagatttgaagaagagctgtttctgaggagccttcaggattacaagattcaaagtgcgctgctggtgccaaccctattctccttcttcgccaaaagcactctga
ttgacaaatacgatttatctaatttacacgaaattgcttctggtggcgctcccctctctaaggaagtcggggaagcggttgccaagaggttccatctgccaggtatc
aggcaaggatatgggctcactgagactacatcagctattctgattacacccgagggggatgataaaccgggcgcggtcggtaaagttgttccattttttgaagcg
aaggttgtggatctggataccgggaaaacgctgggcgttaatcaaagaggcgaactgtgtgtgagaggtcctatgattatgtccggttatgtaaacaatccggaa
gcgaccaacgccttgattgacaaggatggatggctacattctggagacatagcttactgggacgaagacgaacacttcttcatcgttgaccgcctgaagtctctg
attaagtacaaaggctatcaggtggctcccgctgaattggaatccatcttgctccaacaccccaacatcttcgacgcaggtgtcgcaggtcttcccgacgatgac
gccggtgaacttcccgccgccgttgttgttttggagcacggaaagacgatgacggaaaaagagatcgtggattacgtcgccagtcaagtaacaaccgcgaaaa
agttgcgcggaggagttgtgtttgtggacgaagtaccgaaaggtcttaccggaaaactcgacgcaagaaaaatcagagagatcctcataaaggccaagaagg
gcggaaagatcgccgtgtaattctagagtcggggcggccggccgcttcgagcagacatgataagatacattgatgagtttggacaaaccacaactagaatgca
gtgaaaaaaatgctttatttgtgaaatttgtgatgctattgctttatttgtaaccattataagctgcaataaacaagttaacaacaacaattgcattcattttatgtttcaggt
tcagggggaggtgtgggaggttttttaaagcaagtaaaacctctacaaatgtggtaaaatcgataaggatctgaacgatggagcggagaatgggcggaactgg
gcggagttaggggcgggatgggcggagttaggggcgggactatggttgctgactaattgagatgcatgctttgcatacttctgcctgctggggagcctgggga
ctttccacacctggttgctgactaattgagatgcatgctttgcatacttctgcctgctggggagcctggggactttccacaccctaactgacacacattccacagcg
gatccgtcgaccgatgcccttgagagccttcaacccagtcagctccttccggtgggcgcggggcatgactatcgtcgccgcacttatgactgtcttctttatcatg
caactcgtaggacaggtgccggcagcgctcttccgcttcctcgctcactgactcgctgcgctcggtcgttcggctgcggcgagcggtatcagctcactcaaagg
cggtaatacggttatccacagaatcaggggataacgcaggaaagaacatgtgagcaaaaggccagcaaaaggccaggaaccgtaaaaaggccgcgttgct
ggcgtttttccataggctccgcccccctgacgagcatcacaaaaatcgacgctcaagtcagaggtggcgaaacccgacaggactataaagataccaggcgttt
ccccctggaagctccctcgtgcgctctcctgttccgaccctgccgcttaccggatacctgtccgcctttctcccttcgggaagcgtggcgctttctcaatgctcacg
ctgtaggtatctcagttcggtgtaggtcgttcgctccaagctgggctgtgtgcacgaaccccccgttcagcccgaccgctgcgccttatccggtaactatcgtctt
gagtccaacccggtaagacacgacttatcgccactggcagcagccactggtaacaggattagcagagcgaggtatgtaggcggtgctacagagttcttgaagt
ggtggcctaactacggctacactagaaggacagtatttggtatctgcgctctgctgaagccagttaccttcggaaaaagagttggtagctcttgatccggcaaac
aaaccaccgctggtagcggtggtttttttgtttgcaagcagcagattacgcgcagaaaaaaaggatctcaagaagatcctttgatcttttctacggggtctgacgct
cagtggaacgaaaactcacgttaagggattttggtcatgagattatcaaaaaggatcttcacctagatccttttaaattaaaaatgaagttttaaatcaatctaaagtat
atatgagtaaacttggtctgacagttaccaatgcttaatcagtgaggcacctatctcagcgatctgtctatttcgttcatccatagttgcctgactccccgtcgtgtaga
taactacgatacgggagggcttaccatctggccccagtgctgcaatgataccgcgagacccacgctcaccggctccagatttatcagcaataaaccagccagc
cggaagggccgagcgcagaagtggtcctgcaactttatccgcctccatccagtctattaattgttgccgggaagctagagtaagtagttcgccagttaatagtttg
cgcaacgttgttgccattgctacaggcatcgtggtgtcacgctcgtcgtttggtatggcttcattcagctccggttcccaacgatcaaggcgagttacatgatcccc
catgttgtgcaaaaaagcggttagctccttcggtcctccgatcgttgtcagaagtaagttggccgcagtgttatcactcatggttatggcagcactgcataattctctt
actgtcatgccatccgtaagatgcttttctgtgactggtgagtactcaaccaagtcattctgagaatagtgtatgcggcgaccgagttgctcttgcccggcgtcaat
acgggataataccgcgccacatagcagaactttaaaagtgctcatcattggaaaacgttcttcggggcgaaaactctcaaggatcttaccgctgttgagatccagt
tcgatgtaacccactcgtgcacccaactgatcttcagcatcttttactttcaccagcgtttctgggtgagcaaaaacaggaaggcaaaatgccgcaaaaaaggga
ataagggcgacacggaaatgttgaatactcatactcttcctttttcaatattattgaagcatttatcagggttattgtctcatgagcggatacatatttgaatgtatttaga
aaaataaacaaataggggttccgcgcacatttccccgaaaagtgccacctgacgcgccctgtagcggcgcattaagcgcggcgggtgtggtggttacgcgca
gcgtgaccgctacacttgccagcgccctagcgcccgctcctttcgctttcttcccttcctttctcgccacgttcgccggctttccccgtcaagctctaaatcggggg
ctccctttagggttccgatttagtgctttacggcacctcgaccccaaaaaacttgattagggtgatggttcacgtagtgggccatcgccctgatagacggtttttcgc
cctttgacgttggagtccacgttctttaatagtggactcttgttccaaactggaacaacactcaaccctatctcggtctattcttttgatttataagggattttgccgattt
cggcctattggttaaaaaatgagctgatttaacaaaaatttaacgcgaattttaacaaaatattaacgtttacaatttcccattcgccattcaggctgcgcaactgttgg
gaagggcgatcggtgcgggcctcttcgctattacgccagcccaagctaccatgataagtaagtaatattaaggtacgggaggtacttggagcggccgcaataa
aatatctttattttcattacatctgtgtgttggttttttgtgtgaatcgatagtactaacatacgctctccatcaaaacaaaacgaaacaaaacaaactagcaaaataggc
tgtccccagtgcaagtgcaggtgccagaacatttctctatcgata.

### Example 4. Preparation of mRNA-CaLNP Formulations by Different Calcium Acetate Addition Methods

A 400 mmol/l calcium acetate-acetic acid buffer with a pH of 5 was taken as an aqueous phase; 65.0 mg of DLin-MC3-DMA, 31.0 mg of cholesterol, 16.5 mg of DSPC, and 8.0 mg of PEG2000-DMG were weighed and dissolved by adding 10 ml of ethanol to form an organic phase; the organic phase at 4 ml/min and the aqueous phase at 18 ml/min were mixed in a microfluidic chip to collect intermediate I. Intermediate I was dialyzed with a 300 KDa tangential flow membrane, and the dialysate was a 25 mmol/l sodium acetate-acetic acid buffer with pH 4.0. When the volume of the external liquid of dialysis was 200 ml, dialysis was completed, and the volume was made up to the mark to obtain 10 mL of internal liquid as intermediate II. After 0.855 ml of intermediate II was taken, added to 0.200 ml of ethanol, and mixed until uniform, 0.045 ml of 1 mg/ml Firefly Luciferase mRNA (N¹-Me-Pseudo UTP) was added and mixed until uniform, and a 100 mmol/l disodium hydrogen phosphate solution was added to adjust the pH to 7.4 and dialyzed in an ice bath with a PBS buffer with pH 7.4 using a dialysis bag with a molecular weight cut-off of 8000D. The internal liquid was collected, filtered, and sterilized to obtain mRNA-CaLNPs, denoted as 4-1. After measurement, the content was 17 μg/ml, the encapsulation efficiency was 44.30%, and the average particle size was 169.6 nm.

0.4 mg of Firefly Luciferase mRNA (N¹-Me-Pseudo UTP) was taken, and 3.2 ml of a 28 mmol/l sodium acetate-acetic acid buffer with a pH of 4 was added to form aqueous phase 1; 65.0 mg of DLin-MC3-DMA, 31.0 mg of cholesterol, 16.5 mg of DSPC, and 8.0 mg of PEG2000-DMG were weighed and dissolved by adding 10 ml of ethanol to form an organic phase; a 400 mmol/l calcium acetate solution with pH 5 was aqueous phase 2; the organic phase at 4 ml/min and aqueous phase 1 at 18 ml/min were mixed in a microfluidic chip to collect intermediate I. The organic phase at 4 ml/min and aqueous phase 2 at 18 ml/min were mixed in a microfluidic chip to collect intermediate A. 1 ml of intermediate I was taken, added to 1 ml of A, and mixed until uniform to form intermediate II. Intermediate II at 20 ml/min and a 0.4 mol/l Tris buffer with pH 9 at 8 ml/min were mixed using a microfluidic chip, and intermediate III was collected and dialyzed in an ice bath with a Tris buffer with pH 7.4 using a dialysis bag with a molecular weight cut-off of 8000D. The internal phase dialysate was collected, filtered, and sterilized to finally obtain mRNA-CaLNPs, denoted as 4-2. After measurement, the content was 17 μg/ml, the encapsulation efficiency was 97.64%, and the average particle size was 90.8 nm.

0.4 ml of 1 mg/ml Firefly Luciferase mRNA (N¹-Me-Pseudo UTP) was taken, and 3.2 ml of a 28 mmol/l sodium acetate-acetic acid buffer with a pH of 4 was added to form aqueous phase 1; 65.0 mg of DLin-MC3-DMA, 31.0 mg of cholesterol, 16.5 mg of DSPC, and 8.0 mg of PEG2000-DMG were weighed and dissolved by adding 10 ml of ethanol to form an organic phase; a 800 mmol/l calcium acetate-acetic acid solution was aqueous phase 2; and the organic phase at 4 ml/min and aqueous phase 1 at 18 ml/min were mixed in a microfluidic chip to collect intermediate I. Intermediate I at 5 ml/min and aqueous phase 2 at 20 ml/min were mixed using a microfluidic chip, and a finished product was collected and dialyzed in an ice bath with a Tris buffer with pH 7.4 using a dialysis bag with a molecular weight cut-off of 8000D. The internal phase dialysate was collected, filtered, and sterilized to finally obtain calcium-containing mRNA-CaLNPs, denoted as 4-3. After measurement, the content was 9 μg/ml, the encapsulation efficiency was 100%, and the average particle size was 207.4 nm.

0.4 mg of Firefly Luciferase mRNA (N¹-Me-Pseudo UTP) was taken, and 3.2 ml of a 28 mmol/l sodium acetate-acetic acid buffer with a pH of 4 were added to form aqueous phase 1; 65.0 mg of DLin-MC3-DMA, 31.0 mg of cholesterol, 16.5 mg of DSPC, and 8.0 mg of PEG2000-DMG were weighed and dissolved by adding 10 ml of ethanol to form an organic phase; a 400 mmol/l calcium acetate solution with pH 8 was aqueous phase 2; and the organic phase at 4 ml/min and aqueous phase 1 at 18 ml/min were mixed in a microfluidic chip to collect intermediate I. The organic phase at 4 ml/min and aqueous phase 2 at 18 ml/min were mixed in a microfluidic chip to collect intermediate A. 1 ml of intermediate I was taken and mixed with 8 ml of A until uniform to form intermediate II. It was dialyzed in an ice bath with a Tris buffer with pH 7.4 using a dialysis bag with a molecular weight cut-off of 8000D. The internal phase dialysate was collected, filtered, and sterilized to finally obtain mRNA-CaLNPs, denoted as 4-4. After measurement, the content was 19 μg/ml, and the encapsulation efficiency was 60.49%.

### Example 5. Preparation of siRNA-CaLNP Formulations by Different Calcium Acetate Addition Methods

65.0 mg of DLin-MC3-DMA, 31.0 mg of cholesterol, 16.5 mg of DSPC, and 8.0 mg of PEG2000-DMG were weighed and dissolved by adding 10 ml of ethanol to form an organic phase.

1 mg of GAPDH siRNA (Seq No. 1) was taken, and 1 ml of water and 8 ml of a 28 mmol/l sodium acetate-acetic acid buffer with a pH of 4 were added to form aqueous phase 1; a 400 mmol/l calcium acetate-acetic acid solution with pH 5 was aqueous phase 2; and the organic phase at 4 ml/min and aqueous phase 1 at 18 ml/min were mixed in a microfluidic chip to collect intermediate I. The organic phase at 4 ml/min and aqueous phase 2 at 18 ml/min were mixed in a microfluidic chip to collect intermediate A. 1 ml of intermediate I was taken, added to 1 ml of A, and mixed until uniform to form intermediate II, which was dialyzed with a Tris buffer with pH 7.4 using a dialysis bag with a molecular weight cut-off of 8000D. The internal phase dialysate was collected, filtered, and sterilized to finally obtain siRNA-CaLNPs, denoted as 5-1. After measurement, the content was 34 μg/ml, the encapsulation efficiency was 81.61%, and the average particle size was 121.4 nm.

1 mg of siRNA (Seq No. 1) was taken, and 1 ml of water and 8 ml of a 28 mmol/l sodium acetate-acetic acid buffer with a pH of 4 were added to form aqueous phase 1; and the organic phase at 4 ml/min and aqueous phase 1 at 18 ml/min were mixed in a microfluidic chip to collect intermediate I. 30.0 mg of cholesterol and 30 mg of EPC were weighed and dissolved by adding 10 mg of ethanol (10 ml) to form intermediate A, a 400 mmol/l calcium acetate solution was intermediate B, A at 4 ml/min and B at 18 ml/min were mixed in a microfluidic chip, and intermediate II was collected. Intermediate II at 20 ml/min and intermediate I at 2.5 ml/min were mixed using a microfluidic chip, and intermediate III was collected and dialyzed with a Tris buffer with pH 7.4 using a dialysis bag with a molecular weight cut-off of 8000D. The internal phase dialysate was collected, filtered, and sterilized to finally obtain siRNA-CaLNPs, denoted as 5-2. After measurement, the content was 9 μg/ml, the encapsulation efficiency was 83.72%, and the average particle size was 111.8 nm.

1 mg of siRNA (Seq No. 1) was taken, and 1 ml of water and 8 ml of a 28 mmol/l sodium acetate-acetic acid buffer with a pH of 4 were added to form aqueous phase 1; and a 400 mmol/l acetic acid solution was aqueous phase 2; and the organic phase at 4 ml/min and aqueous phase 1 at 18 ml/min were mixed in a microfluidic chip to collect intermediate I. Intermediate I at 2.5 ml/min and aqueous phase 2 at 16.3 ml/min were mixed using a microfluidic chip, and a finished product was collected and dialyzed in an ice bath with a Tris buffer with pH 7.4 using a dialysis bag with a molecular weight cut-off of 8000D. The internal phase dialysate was collected, filtered, and sterilized to finally obtain siRNA-CaLNPs, denoted as 5-3. After measurement, the content was 31 μg/ml, the encapsulation efficiency was 91.16%, and the average particle size was 88.7 nm.

1 mg of siRNA (Seq No. 1) was taken, and 1 ml of water and 8 ml of a 28 mmol/l sodium acetate-acetic acid buffer with a pH of 4 were added to form aqueous phase 1; a 800 mmol/l calcium acetate solution was aqueous phase 2; and the organic phase at 4 ml/min and aqueous phase 1 at 18 ml/min were mixed in a microfluidic chip to collect intermediate I. Intermediate I at 2.5 ml/min and aqueous phase 2 at 10 ml/min were mixed using a microfluidic chip, and a finished product was collected and dialyzed in an ice bath with a Tris buffer with pH 7.4 using a dialysis bag with a molecular weight cut-off of 8000D. The internal phase dialysate was collected, filtered, and sterilized to finally obtain siRNA-CaLNPs, denoted as 5-4. After measurement, the content was 37.11 μg/ml, the encapsulation efficiency was 97.56%, and the average particle size was 76.4 nm.

TABLE 4
Detection data of cholesterol and DSPC
in each formulation in Example 5

| | Cholesterol | DSPC concentration, |
|---|---|---|
| Formulation code | concentration, μg/ml | μg/ml |
| 5-1 | 277.99 | 165.62 |
| 5-2 | 363.351 | 48.41 |
| 5-3 | 106.63 | 57.73 |
| 5-4 | 114.56 | 67.14 |

### Example 6. To Prepare Calcium-Free siRNA-LNP Formulation as Compared with Calcium-Containing Formulation

65.0 mg of DLin-MC3-DMA, 31.0 mg of cholesterol, 16.5 mg of DSPC, and 8.0 mg of PEG2000-DMG were weighed and dissolved by adding 10 ml of ethanol to form an organic phase; 0.1 mg of GAPDH siRNA (Seq No. 1) was taken, added to 0.9 ml of a 28 mmol/l sodium acetate-acetic acid solution with pH 4, and mixed until uniform to form an aqueous phase; and 0.9 ml of the aqueous phase and 0.2 ml of the organic phase were taken and mixed manually until uniform to obtain intermediate I, which was dialyzed in a Tris-PBS buffer with pH 7.4 by a dialysis bag with a molecular weight cut-off of 8000D. The internal phase dialysate was collected, filtered, and sterilized to finally obtain calcium-free siRNA-LNPs, denoted as 6-1. After measurement, the content was 57 μg/ml, the encapsulation efficiency was 97.07%, and the average particle size was 102.6 nm.

TABLE 5
Detection data of cholesterol and DSPC
in each formulation in Example 6

| | Cholesterol | DSPC concentration, |
|---|---|---|
| Formulation code | concentration, μg/ml | μg/ml |
| 6-1 | 258.04 | 140.61 |

### Example 7. To Prepare Calcium-Free mRNA-LNP Formulation as Compared with Calcium-Containing Formulation

0.4 ml of 1 mg/ml Firefly Luciferase mRNA (N¹-Me-Pseudo UTP) was taken, and 3.2 ml of a 28 mmol/l sodium acetate-acetic acid buffer with a pH of 4 was added to form an aqueous phase; 65.0 mg of DLin-MC3-DMA, 31.0 mg of cholesterol, 16.5 mg of DSPC, and 8.0 mg of PEG2000-DMG were weighed and dissolved by adding 10 ml of ethanol to form an organic phase; the organic phase at 4 ml/min and an aqueous phase at 18 ml/min were mixed in a microfluidic chip to collect intermediate I. It was dialyzed in an ice bath with a PBS buffer with pH 7.4 using a dialysis bag with a molecular weight cut-off of 8000D. The internal phase dialysate was collected, filtered, and sterilized to finally obtain calcium-free mRNA-LNPs, denoted as 7-1. After measurement, the content was 26 μg/ml, the encapsulation efficiency was 96.82%, and the average particle size was 96.9 nm.

### Example 8. To Prepare Calcium-Free DNA-LNP Formulation as Compared with Calcium-Containing Formulation

65.0 mg of DLin-MC3-DMA, 31.0 mg of cholesterol, 16.5 mg of DSPC, and 8.0 mg of PEG2000-DMG were weighed and dissolved by adding 10 ml of ethanol to form an organic phase; 1.0 mg/ml pGL3-control plasmid was taken, added to 8 ml of a 28 mmol/l sodium acetate-acetic acid buffer with pH 4.0, and mixed until uniform to form an aqueous phase; and the organic phase at 4 ml/min and the aqueous phase at 18 ml/min were mixed in a microfluidic chip to collect intermediate I. It was dialyzed in an ice bath with a PBS buffer with pH 7.4 using a dialysis bag with a molecular weight cut-off of 8000D. The internal phase dialysate was collected, filtered, and sterilized to finally obtain calcium-free DNA-LNPs, denoted as 8-1. After measurement, the content was 54 μg/ml, the encapsulation efficiency was 97.22%, and the average particle size was 106.2 nm.

### Example 9. Preparation of siRNA-CaLNPs with Different Ionizable Cationic Materials and Neutral Lipid Materials

65.0 mg of DLinDMA, 31.0 mg of cholesterol, 16.5 mg of DSPC, and 8.0 mg of PEG2000-DMG were weighed and dissolved by adding 10 ml of ethanol to form an organic phase; 0.1 mg of GAPDH siRNA (Seq. No. 1) was taken, added to 0.9 ml of a 200 mmol/l calcium acetate solution with pH 5, and mixed until uniform to form an aqueous phase; and 0.9 ml of the aqueous phase and 0.2 ml of the organic phase were taken and mixed manually until uniform to obtain an intermediate, which was dialyzed in a Tris buffer with pH 7.4 by a dialysis bag with a molecular weight cut-off of 8000D. The internal phase dialysate was collected, filtered, and sterilized to finally obtain siRNA-CaLNPs, denoted as 9-1. After measurement, the content was 17 μg/ml, the encapsulation efficiency was 77.54%, and the average particle size was 268.7 nm. 0.1 mg of GAPDH siRNA was taken, added to 0.9 ml of a 25 mmol/l sodium acetate solution with pH 4, and mixed until uniform to form an aqueous phase; 0.9 ml of the aqueous phase and 0.2 ml of the organic phase were taken and mixed manually until uniform to obtain an intermediate, which was dialyzed in a Tris buffer with pH 7.4 by a dialysis bag with a molecular weight cut-off of 8000D. The internal phase dialysate was collected, filtered, and sterilized to finally obtain siRNA-LNPs, denoted as 9-2. After measurement, the content was 36 μg/ml, the encapsulation efficiency was 92.64%, and the average particle size was 91.94 nm.

65.0 mg of DODMA, 31.0 mg of cholesterol, 16.5 mg of DSPC, and 8.0 mg of PEG2000-DMG were dissolved by adding 10 ml of ethanol to form an organic phase; 0.1 mg of GAPDH siRNA (Seq. No. 1) was taken, added to 0.9 ml of a 200 mmol/l calcium acetate solution with pH 5, and mixed until uniform to form an aqueous phase; and 0.9 ml of the aqueous phase and 0.2 ml of the organic phase were taken and mixed manually until uniform to obtain an intermediate, which was dialyzed in a Tris buffer with pH 7.4 by a dialysis bag with a molecular weight cut-off of 8000D. The internal phase dialysate was collected, filtered, and sterilized to finally obtain siRNA-CaLNPs, denoted as 9-3. After measurement, the content was 33 μg/ml, the encapsulation efficiency was 83.93%, and the average particle size was 139.7 nm. 0.1 mg of GAPDH siRNA (Seq. No. 1) was taken, added to 0.9 ml of a 25 mmol/l sodium acetate solution with pH 4, and mixed until uniform to form an aqueous phase; and 0.9 ml of the aqueous phase and 0.2 ml of the organic phase were taken and mixed manually until uniform to obtain an intermediate, which was dialyzed in a Tris buffer with pH 7.4 by a dialysis bag with a molecular weight cut-off of 8000D. The internal phase dialysate was collected, filtered, and sterilized to finally obtain siRNA-LNPs, denoted as 9-4. After measurement, the content was 36 μg/ml, the encapsulation efficiency was 88.88%, and the average particle size was 91.91 nm.

65.0 mg of DLin-MC3-DMA, 31.0 mg of cholesterol, 16.5 mg of DOPE, and 8.0 mg of PEG2000-DMG were weighed and dissolved by adding 10 ml of ethanol to form an organic phase; 0.1 mg of GAPDH siRNA (Seq. No. 1) was taken, added to 0.9 ml of a 200 mmol/l calcium acetate solution with pH 5, and mixed until uniform to form an aqueous phase; and 0.9 ml of the aqueous phase and 0.2 ml of the organic phase were taken and mixed manually until uniform to obtain an intermediate, which was dialyzed in a Tris buffer with pH 7.4 by a dialysis bag with a molecular weight cut-off of 8000D. The internal phase dialysate was collected, filtered, and sterilized to finally obtain siRNA-CaLNPs, denoted as 9-5. After measurement, the content was 32 μg/ml, the encapsulation efficiency was 95.83%, and the average particle size was 105.4 nm. 0.1 mg of GAPDH siRNA was taken, added to 0.9 ml of a 25 mmol/l sodium acetate solution with pH 4, and mixed until uniform to form an aqueous phase; 0.9 ml of the aqueous phase and 0.2 ml of the organic phase were taken and mixed manually until uniform to obtain an intermediate, which was dialyzed in a Tris buffer with pH 7.4 by a dialysis bag with a molecular weight cut-off of 8000D. The internal phase dialysate was collected, filtered, and sterilized to finally obtain siRNA-LNPs, denoted as 9-6. After measurement, the content was 32 μg/ml, the encapsulation efficiency was 93.37%, and the average particle size was 93.38 nm.

65.0 mg of DLin-MC3-DMA, 31.0 mg of cholesterol, 16.5 mg of DOPC, and 8.0 mg of PEG2000-DMG were weighed and dissolved by adding 10 ml of ethanol to form an organic phase; 0.1 mg of GAPDH siRNA (Seq. No. 1) was taken, added to 0.9 ml of a 200 mmol/l calcium acetate solution with pH 5, and mixed until uniform to form an aqueous phase; and 0.9 ml of the aqueous phase and 0.2 ml of the organic phase were taken and mixed manually until uniform to obtain an intermediate, which was dialyzed in a Tris buffer with pH 7.4 by a dialysis bag with a molecular weight cut-off of 8000D. The internal phase dialysate was collected, filtered, and sterilized to finally obtain siRNA-CaLNPs, denoted as 9-7. After measurement, the content was 33 μg/ml, the encapsulation efficiency was 94.83%, and the average particle size was 87.43 nm. 0.1 mg of GAPDH siRNA was taken, added to 0.9 ml of a 25 mmol/l sodium acetate solution with pH 4, and mixed until uniform to form an aqueous phase; 0.9 ml of the aqueous phase and 0.2 ml of the organic phase were taken and mixed manually until uniform to obtain an intermediate, which was dialyzed in a Tris buffer with pH 7.4 by a dialysis bag with a molecular weight cut-off of 8000D. The internal phase dialysate was collected, filtered, and sterilized to finally obtain siRNA-LNPs, denoted as 9-8. After measurement, the content was 36 μg/ml, the encapsulation efficiency was 94.75%, and the average particle size was 74.86 nm.

### Example 10. Preparation of siRNA-CaLNP Formulations with Different Concentrations of Calcium Acetate

32.5 mg of DLin-MC3-DMA, 31.0 mg of cholesterol, 16.5 mg of DSPC, and 8.0 mg of PEG-DMG were weighed and dissolved by adding 10 ml of ethanol to form an organic phase; 0.2 mg of siRNA (human GAPDH) was taken separately and diluted with 1.8 mL of calcium acetate solutions with different concentrations to form aqueous phases, wherein the pH value of the calcium acetate solutions with different concentrations was adjusted to 5 with acetic acid; and 1.8 mL of the aqueous phase and 0.4 ml of the organic phase were taken and mixed into a tee pipe at a total flow rate of 22 mL/min to obtain intermediate I, which was dialyzed in a Tris-containing buffer with pH 7.2 by a dialysis bag with a molecular weight cut-off of 14000D. The internal phase dialysate was collected, filtered, and sterilized to finally obtain siRNA-CaLNPs. The different concentrations of the calcium acetate solutions in Table 6 were 25 mmol/L, 50 mmol/L, 100 mmol/L, 200 mmol/L, and 400 mmol/L.

TABLE 6
Properties of siRNA-CaL NP formulations in Example 10

| | Ca(Ac) | | | | | | | | | | | |
|---|---|---|---|---|---|---|---|---|---|---|---|---|
| | concentration | | | Particle | | | | Free Ca²⁺ | Total Ca²⁺ | ΔCa²⁺ | | |
| Formulation | in aqueous | | | size | | Content | Encapsulation | concentration, | concentration, | concentration, | Chol | ΔCa²⁺/ |
| code | phase system | | N/P | nm | PDI | μg/ml | efficiency, % | μM | μM | μM | μM | Chol |
| 10-1 | 25 | mM | | 56 | 0.192 | 77.00 | 98.2 | 46.5 | 68.6 | 22.1 | 745.0 | 0.030 |
| 10-2 | 50 | mM | | 64 | 0.236 | 73.00 | 98.1 | 51.7 | 902 | 38.6 | 683.5 | 0.056 |
| 10-3 | 100 | mM | | 70 | 0.149 | 69.00 | 98.3 | 68.9 | 133.5 | 64.5 | 578.6 | 0.111 |
| 10-4 | 200 | mM | 3 | 113 | 0.119 | 77.00 | 97.7 | 80.0 | 208.5 | 128.5 | 505.4 | 0.254 |
| 10-5 | 400 | mM | 3 | 146 | 0.093 | 57.00 | 88.4 | 159 | 286.1 | 127.1 | 446.8 | 0.284 |

### Example 11. Preparation of siRNA-CaLNP Formulations from Calcium Acetate Solutions with Different pH Values

32.5 mg of DLin-MC3-DMA, 31.0 mg of cholesterol, 16.5 mg of DSPC, and 8.0 mg of PEG-DMG were weighed and dissolved by adding 10 ml of ethanol to form an organic phase; 0.2 mg of siRNA (human GAPDH gene) was taken separately and diluted with 1.8 ml of a 200 mmol/l calcium acetate solution to form an aqueous phase, wherein the calcium acetate solution was adjusted to different pH values with acetic acid; and 1.8 ml of the aqueous phase and 0.4 ml of the organic phase were taken and mixed into a tee pipe at a total flow rate of 22 ml/min to obtain intermediate I, which was dialyzed in a Tris-containing buffer with pH 7.2 by a dialysis bag with a molecular weight cut-off of 14000D. The internal phase dialysate was collected, filtered, and sterilized to finally obtain siRNA-CaLNPs. The different pH values of the 200 mmol/l calcium acetate solutions in Table 7 were pH 4.5, pH 4.8, pH 5.0, pH 5.2, and pH 5.5.

TABLE 7
Properties of siRNA-CaLNP formulations in this study

| Formulation | pH | | Particle | | Content, | Encapsulation |
|---|---|---|---|---|---|---|
| code | value | N/P | size, nm | PDI | μg/ml | efficiency |
| 11-1 | 4.5 | 3 | 119.5 | 0.072 | 54.29 | 94.89% |
| 11-2 | 4.8 | 3 | 122.7 | 0.062 | 55.90 | 95.63% |
| 11-3 | 5.0 | 3 | 107.7 | 0.065 | 55.50 | 95.49% |
| 11-4 | 5.2 | 3 | 100.9 | 0.058 | 56.71 | 94.43% |
| 11-5 | 5.5 | 3 | 108.1 | 0.077 | 50.27 | 93.78% |

By means of preparation under different pH value conditions, it could be known that the applicable pH value range for the CaLNPs was wide, and good preparation results could be obtained even under acidic conditions (e.g., pH 4.5-5.5).

### Example 12. Preparation of siRNA-CaLNP Formulations with Different N/P Ratios

32.5 mg of DLin-MC3-DMA, 31.0 mg of cholesterol, 16.5 mg of DSPC, and 8.0 mg of PEG-DMG were weighed and dissolved by adding 10 ml of ethanol to form an organic phase; different amounts of siRNA (human GAPDH gene) were separately taken and diluted with 1.8 mL of a 200 mmol/L calcium acetate solution (pH 5.0) and a 400 mmol/L calcium acetate solution (pH 5.0) to form aqueous phases, wherein the pH value of the calcium acetate solution was adjusted with acetic acid. 1.8 mL of the aqueous phase and 0.4 ml of the organic phase were taken and mixed into a tee pipe at a total flow rate of 22 mL/min to obtain intermediate I, which was dialyzed in a Tris-containing buffer with pH 7.2 by a dialysis bag with a molecular weight cut-off of 14000D. The internal phase dialysate was collected, filtered, and sterilized to finally obtain siRNA-CaLNPs. The amounts of siRNA in Table 8 were 0.1 mg, 0.2 mg, and 0.4 mg, respectively.

TABLE 8
LNPs prepared with the same calcium acetate ion concentration and different N/P ratios

| | Ca(Ac)₂ | | | | | | | | | | |
|---|---|---|---|---|---|---|---|---|---|---|---|
| Formula- | concentration | | Particle | | | | Free Ca²⁺ | Total Ca²⁺ | ΔCa²⁺ | | |
| tion | in aqueous | | size, | | Content | Encapsulation | concentration, | concentration, | concentration, | | |
| code | phase system | N/P | nm | PDI | μM/ml | efficiency | μM | μM | μM | Chol | ΔCa²⁺/Chol |
| 12-1 | 200 mM | 1.5 | 138 | 0.105 | 137.00 | 90.6% | 170.8 | 320.5 | 149.8 | 424.4 | 0.353 |
| 12-2 | 200 mM | 3 | 113 | 0.119 | 77.00 | 97.7% | 80.0 | 208.5 | 128.5 | 505.4 | 0.254 |
| 12-3 | 200 mM | 6 | 83 | 0.175 | 32.25 | 97.8% | 149.8 | 232.7 | 82.9 | 584.7 | 0.142 |
| 12-4 | 400 mM | 3 | 146 | 0.093 | 57.00 | 88.4% | 159.0 | 286.1 | 127.1 | 446.8 | 0.284 |
| 12-5 | 400 mM | 6 | 111 | 0.058 | 33.25 | 93.2% | 178.5 | 353.2 | 174.7 | 456.2 | 0.383 |

### Example 13. Preparation of siRNA-CaLNP Formulations with Different Types of Cationic Lipids

Different types of cationic lipids, 31.0 mg of cholesterol, 16.5 mg of DSPC, and 8.0 mg of (human GAPDH gene) was taken separately and diluted with 1.8 ml of a 200 mmol/l calcium acetate solution (pH 5.0) to form an aqueous phase, wherein the pH value of the calcium acetate solution was adjusted with acetic acid; and 1.8 ml of the aqueous phase and 0.4 ml of the organic phase were taken and mixed into a tee pipe at a total flow rate of 22 ml/min to obtain intermediate I, which was dialyzed in a Tris-containing buffer with pH 7.2 by a dialysis bag with a molecular weight cut-off of 14000D. The internal phase dialysate was collected, filtered, and sterilized to finally obtain siRNA-CaLNPs. The types and amounts of the cations in Table 9 were Dlin-MC3-DMA 32.5 mg, Dlin-DMA 32.5 mg, SM102 35.9 mg, and DOTAP 35.0 mg, respectively.

TABLE 9
Preparation of siRNA-CaLNP formulations with different types of cationic lipids

| Formula- | | | | | | Free Ca²⁺ | Total Ca²⁺ | ΔCa²⁺ | Chol | |
|---|---|---|---|---|---|---|---|---|---|---|
| tion | Lipid | Particle | | Content, | Encapsulation | concentration. | concentration, | concentration, | concentration, | |
| code | type | size, nm | PDI | μg/ml | efficiency | μM | μM | μM | μM | ΔCa²⁺/Chol |
| 13-1 | MC3 | 113 | 0.119 | 77.00 | 97.7% | 80.0 | 208.5 | 128.5 | 505.4 | 0.254 |
| 13-2 | DlinDMA | 110 | 0.083 | 44.66 | 98.7% | 98.8 | 143.7 | 44.9 | 727.0 | 0.062 |
| 13-3 | SM102 | 151 | 0.532 | 47.39 | 95.3% | 221.3 | 253.8 | 32.6 | 366.1 | 0.089 |
| 13-4 | DOTAP | 102 | 0.339 | 35.18 | 97.6% | 248.1 | 334.0 | 85.9 | 674.7 | 0.127 |

### Example 14. Preparation of siRNA-CaLNP Formulations with Different Amounts of Cationic Lipids

Different amounts of Dlin-MC3-DMA, 31.0 mg of cholesterol, 16.5 mg of DSPC, and 8.0 mg of PEG-DMG were weighed and dissolved by adding 10 ml of ethanol to form organic phases; Different amounts of siRNA (human GAPDH gene) were taken separately and diluted with 1.8 ml of a 200 mmol/l calcium acetate solution (pH 5.0) to form aqueous phases, wherein the pH value of the calcium acetate solution was adjusted with acetic acid; and 1.8 ml of the aqueous phase and 0.4 ml of the organic phase were taken and mixed into a tee pipe at a total flow rate of 22 ml/min to obtain intermediate I, which was dialyzed in a Tris-containing buffer with pH 7.2 by a dialysis bag with a molecular weight cut-off of 14000D. The internal phase dialysate was collected, filtered, and sterilized to finally obtain siRNA-CaLNPs. The amounts of Dlin-MC3-DMA and siRNA in Table 10 were Dlin-MC3-DMA 65 mg and siRNA 0.4 mg, Dlin-MC3-DMA 32.5 mg and siRNA 0.2 mg, and Dlin-MC3-DMA 16.3 mg and siRNA 0.1 mg, respectively.

TABLE 10
Preparation of siRNA-CaLNP formulations with different amounts of cationic lipids

| | Amounts of | | | | | Free Ca²⁺ | Total Ca²⁺ | ΔCa²⁺ | |
|---|---|---|---|---|---|---|---|---|---|
| Formulation | MC3 and | Particle | | Content, | Encapsulation | concentration, | concentration, | concentration, | Chol |
| code | siRNA | size, nm | PDI | μg/ml | efficiency | μM | μM | μM | μM |
| 14-1 | MC3.65 mg | 156 | 0.357 | 93.27 | 88.7% | 89.1 | 139.1 | 49.9 | 627.0 |
| | siRNA 0.4 mg | | | | | | | | |
| 14-2 | MC3 32.5 mg | 113 | 0.119 | 77.00 | 97.7% | 80.0 | 208.5 | 128.5 | 505.0 |
| | siRNA 0.2 mg | | | | | | | | |
| 14-3 | MC3 16.3 mg | 167 | 0.053 | 24.93 | 81.7% | — | — | — | — |
| | siRNA 0.1 mg | | | | | | | | |

### Example 15. Preparation of siRNA-CaLNP Formulations with Different Amounts of Neutral Lipids

32.5 mg of DLin-MC3-DMA, 31.0 mg of cholesterol, different amounts of DSPC, and 8.0 mg of (human GAPDH gene) was taken separately and diluted with 1.8 ml of a 200 mmol/l calcium acetate solution (pH 5.0) to form an aqueous phase, wherein the pH value of the calcium acetate was adjusted to 5 with acetic acid; and 1.8 ml of the aqueous phase and 0.4 ml of the organic phase were taken and mixed into a tee pipe at a total flow rate of 22 ml/min to obtain intermediate I, which was dialyzed in a Tris-containing buffer with pH 7.2 by a dialysis bag with a molecular weight cut-off of 14000D. The internal phase dialysate was collected, filtered, and sterilized to finally obtain siRNA-CaLNPs. The different amounts of DSPC in Table 11 were 33.0 mg, 16.5 mg, 8.3 mg, and 0 mg, respectively.

TABLE 11
Preparation of siRNA-CaLNP formulations with different amounts of neutral lipids

| | Amount | | | | | Free Ca²⁺ | Total Ca²⁺ | ΔCa²⁺ | |
|---|---|---|---|---|---|---|---|---|---|
| Formulation | of | Particle | | Content, | Encapsulation | concentration, | concentration, | concentration, | Chol |
| code | DSPC | size, nm | PDI | μg/ml | efficiency | μM | μM | μM | μM |
| 15-1 | 33.0 mg | 74 | 0.178 | 45.98 | 68.9% | 77.9 | 93.8 | 16.0 | 163.5 |
| 15-2 | 16.5 mg | 113 | 0.119 | 77.00 | 97.7% | 80.0 | 208.5 | 128.5 | 505.4 |
| 15-3 | 8.3 mg | 180 | 0.179 | 54.84 | 95.6% | 74.6 | 158.0 | 83.4 | 812.2 |
| 15-4 | 0 | 259 | 0.202 | 49.92 | 61.2% | 87.5 | 113.2 | 25.6 | 727.0 |

### Example 16. Preparation of siRNA-CaLNP Formulations with Different Amounts of Cholesterol

32.5 mg of DLin-MC3-DMA, different amounts of cholesterol, 16.5 mg of DSPC, and 8.0 mg of (human GAPDH gene) was taken separately and diluted with 1.8 ml of a 200 mmol/l calcium acetate solution (pH 5.0) to form an aqueous phase, wherein the pH value of the calcium acetate was adjusted to 5 with acetic acid; and 1.8 ml of the aqueous phase and 0.4 ml of the organic phase were taken and mixed into a tee pipe at a total flow rate of 22 ml/min to obtain intermediate I, which was dialyzed in a Tris-containing buffer with pH 7.2 by a dialysis bag with a molecular weight cut-off of 14000D. The internal phase dialysate was collected, filtered, and sterilized to finally obtain siRNA-CaLNPs. The different amounts of Chol in Table 12 were 62.0 mg, 31.0 mg, 15.5 mg, and 0 mg, respectively.

TABLE 12
Prepation of siRNA-CaLNP formulations with different amounts of cholesterol

| | | | | | | Free Ca²⁺ | Total Ca²⁺ | ΔCa²⁺ | |
|---|---|---|---|---|---|---|---|---|---|
| Formulation | Amount | Particle | | Content, | Encapsulation | concentration, | concentration, | concentration, | Chol |
| code | of Chol | size, nm | PDI | μg/ml | efficiency | μM | μM | μM | μM |
| 16-1 | 62.0 mg | 323 | 0.295 | 47.39 | 93.7% | 278.3 | 406.2 | 127.9 | 1134.4 |
| 16-2 | 31.0 mg | 113 | 0.119 | 77.00 | 97.7% | 80.0 | 208.5 | 128.5 | 505.4 |
| 16-3 | 15.5 mg | 93 | 0.335 | 40.30 | 94.4% | 235.5 | 266.4 | 30.8 | 375.8 |
| 16-4 | 0 | — | — | 64.47 | 8.9% | — | — | — | — |

### Example 17. Preparation of siRNA-CaLNP Formulations with Different Amounts of PEG Lipids

32.5 mg of DLin-MC3-DMA, 31.0 mg of cholesterol, 16.5 mg of DSPC, and different amounts of (human GAPDH gene) was taken separately and diluted with 1.8 ml of a 200 mmol/l calcium acetate solution (pH 5.0) to form an aqueous phase, wherein the pH value of the calcium acetate was adjusted to 5 with acetic acid; and 1.8 ml of the aqueous phase and 0.4 ml of the organic phase were taken and mixed into a tee pipe at a total flow rate of 22 ml/min to obtain intermediate I, which was dialyzed in a Tris-containing buffer with pH 7.2 by a dialysis bag with a molecular weight cut-off of 14000D. The internal phase dialysate was collected, filtered, and sterilized to finally obtain siRNA-CaLNPs. The different amounts of PEG-DMG in Table 13 were 16.0 mg, 8.0 mg, 4.0 mg, and 0 mg, respectively.

TABLE 13
Preparation of siRNA-CaLNP formulations with different amounts of PEG lipids

| | | | | | | Free Ca²⁺ | Total Ca²⁺ | ΔCa²⁺ | |
|---|---|---|---|---|---|---|---|---|---|
| Formulation | | Particle | | Content, | Encapsulation | concentration, | concentration, | concentration, | Chol |
| code | PEG-DMG | size, nm | PDI | μg/ml | efficiency | μM | μM | μM | μM |
| 17-1 | 16.0 mg | 75 | 0.169 | 56.16 | 93.1% | 95.9 | 166.5 | 70.6 | 705.4 |
| 17-2 | 8.0 mg | 113 | 0.119 | 77.00 | 97.7% | 80.0 | 208.5 | 128.5 | 505.4 |
| 17-3 | 4.0 mg | 71 | 0.086 | 51.23 | 94.9% | 102.4 | 194.8 | 92.4 | 555.4 |
| 17-4 | non | 122 | 0.228 | 35.80 | 94.2% | 155.2 | 197.5 | 42.3 | 502.7 |

### Example 18. Preparation of siRNA-LNP Formulations with Different Types of Cationic Lipids

Different types of cationic lipids, 31.0 mg of cholesterol, 16.5 mg of DSPC, and 8.0 mg of PEG-DMG were weighed and dissolved by adding 10 ml of ethanol to form organic phases; different amounts of siRNA (human GAPDH gene) were taken separately and diluted with 1.8 ml of a 25 mmol/L acetic acid-sodium acetate solution (pH 4.0) to form aqueous phases, wherein the pH value of the 25 mmol/L acetic acid-sodium acetate solution was adjusted to 4.0 with acetic acid; and 1.8 ml of the aqueous phase and 0.4 ml of the organic phase were taken and mixed into a tee pipe at a total flow rate of 22 ml/min to obtain intermediate I, which was dialyzed in a PBS-containing buffer with pH 7.2 by a dialysis bag with a molecular weight cut-off of 14000D. The internal phase dialysate was collected, filtered, and sterilized to finally obtain the siRNA-LNP. The different types of cationic lipids and different amounts of siRNA in Table 14 were Dlin-MC3-DMA 65 mg and 0.4 mg, Dlin-MC3-DMA 32.5 mg and 0.2 mg, Dlin-DMA 65 mg and 0.4 mg, Dlin-DMA 32.5 mg and 0.2 mg, SM102 71.8 mg and 0.4 mg, SM102 35.9 mg and 0.2 mg, DOTAP 70.0 mg and 0.4 mg, and DOTAP 35.0 mg and 0.2 mg, respectively.

TABLE 14
Preparation of siRNA-LNP formulations with different types of cationic lipids

| Formulation | | | Particle | | Content, | Encapsulation |
|---|---|---|---|---|---|---|
| code | Lipid | siRNA | size, nm | PDI | μg/ml | efficiency |
| 18-1 | Dlin-MC3-DMA | 0.4 mg | 94 | 0.097 | 113.30 | 96.3% |
| | 65 mg | | | | | |
| 18-2 | Dlin-MC3-DMA | 0.2 mg | 93 | 0.394 | 57.80 | 95.9% |
| | 32.5 mg | | | | | |
| 18-3 | Dlin-DMA | 0.4 mg | — | — | 115.59 | 94.0% |
| | 65 mg | | | | | |
| 18-4 | Dlin-DMA | 0.2 mg | 82 | 0.201 | 67.75 | 92.1% |
| | 32.5 mg | | | | | |
| 18-5 | SM102 | 0.4 mg | 92 | 0.073 | 96.46 | 97.0% |
| | 71.8 mg | | | | | |
| 18-6 | SM102 | 0.2 mg | 93 | 0.265 | 48.61 | 95.1% |
| | 35.9 mg | | | | | |
| 18-7 | DOTAP | 0.4 mg | 99 | 0.186 | 87.27 | 98.6% |
| | 70.0 mg | | | | | |
| 18-8 | DOTAP | 0.2 mg | 102 | 0.339 | 35.18 | 97.6% |
| | 35.0 mg | | | | | |

Formulations with Formulation codes 10-4, 12-2, 13-1, 14-2, 15-2, 16-2, and 17-2 were the same batch of formulations, which were denoted with different codes in different examples.

### Experimental Example 1. Experiment of Transfection of Cells with GAPDH siRNA

### Cell Culturing:

HepG2, a human hepatocarcinoma cell line, was left to stand and cultured in 90% DMEM medium+10% fetal bovine serum culture+1% Penicillin-Streptomycin medium at 5% CO₂ at 37° C. and observed under an inverted microscope, and the cells in the logarithmic growth phase were selected, digested with 0.25% trypsin, counted, and plated. (1) The medium was discarded, and after washing once with 2 ml of a PBS solution, the PBS was discarded. (2) 1 ml of 0.25% trypsin was added, spread evenly up and down and left and right, digested at 37° C. for 10 min, and observed at any time, and when the cells flowed down in a muddy state, the digestion was complete. (3) 1 ml of a medium was added, the digestion was terminated, the digested cells were repeatedly bubbled until they were detached and dispersed to form a cell suspension, which was centrifuged, and the supernatant was discarded. (4) The cells were re-suspended with the medium and counted on a counting plate. Each 6-well plate with 2 ml of the medium was inoculated at 300,000 cells per well, and after culturing for 24 hours until the cells had grown to 70-80%, dosage was started.

### Dosage and Cell Treatment:

When the cells had grown to 70-80%, dosage was carried out. Positive control (100 nM-siRNA, Seq. No. 1): 5.4 μl of 1 mg/ml siRNA+8 μl of lipofectamin 3000 solution was made up to 200 μl by adding a serum-free DMEM culture solution and serially diluted into different concentrations; Negative control (directly adding PBS); each agent was separately prepared with serum-free DMEM to the dosage concentration; and after the agent was prepared, 100 μl was added to the corresponding 6-well plate. Culturing was continued for 24 hours. The cells in the 6-well plate were digested, collected, and detected by RT-qPCR.

### RT-qPCR:

RNA was extracted by Beyotime RNAeasy™ animal RNA extraction kit (centrifugal column-type), and BeyoFast™ SYBR Green One-step qRT-PCR Kit was used for one-step reverse transcription real-time fluorescence quantitative PCR.

(1) RNA extraction: The collected cells were centrifuged at 1500 g, the supernatant was discarded, 1 ml of PBS was added, the mixture was centrifuged at 1500 g, the supernatant was discarded, 0.3 ml of a lysis liquid was added, the mixture was vortexed, 0.3 ml of a binding solution was added, and the mixture was vortexed.

The mixture was transferred to a purification column and centrifuged at 12000 g for 30 s, and the liquid in the tube was discarded;
600 μl of washing liquid 1 was added, and after centrifugation at 12000 g for 30 s, the liquid in the tube was discarded;
600 μl of washing liquid 2 was added, and after centrifugation at 12000 g for 30 s, the liquid in the tube was discarded, and this operation was repeated once;
centrifugation was carried out at the highest speed of 14000 g for 2 minutes to remove the residual liquid; and
the lower centrifuge tube was discarded, and an RNA elution tube was used as replacement, 50 μl of an eluent (added to a groove as much as possible) was added, and it was left at room temperature for 2 minutes and centrifuged at 14000 g for 2 minutes. The obtained solution was purified RNA.

(2) Template preparation: The extracted RNA was diluted 50 times with RNase-free water.

(3) primer preparation: A: the dosages of each sample (20 μl) were respectively: 10 μl of SYBR Green One-Step Reaction Buffer (2×); 2 μl of SYBR Green One-Step Enzyme Mix (10×); 0.4 μl of Low ROX (50×); 3.6 μl of RNase-free water; 2 μl of a template sample; and 2 μl of primers containing GAPDH-F and GAPDH-R (GAPDH-F sequence: CTTCTTTTGCGTCGCCAGCC, and GAPDH-R sequence: GTTCTCAGCCTTGACGGTGC) each at 3 nmol/ml.

B: The dosages of each sample (20 μl) were respectively 10 μl of SYBR Green One-Step Reaction Buffer (2×); 2 μl of SYBR Green One-Step Enzyme Mix (10×); 0.4 μl of Low ROX (50×); 3.6 μl of RNase-free water; 2 μl of a template sample; and 2 μl of primers containing β-actin-F and β-actin-R (β-actin-F sequence: CCTGGCACCCAGCACAAT, and β-actin-R sequence: GGGCCGGACTCGTCATAC) each at 3 nmol/ml.

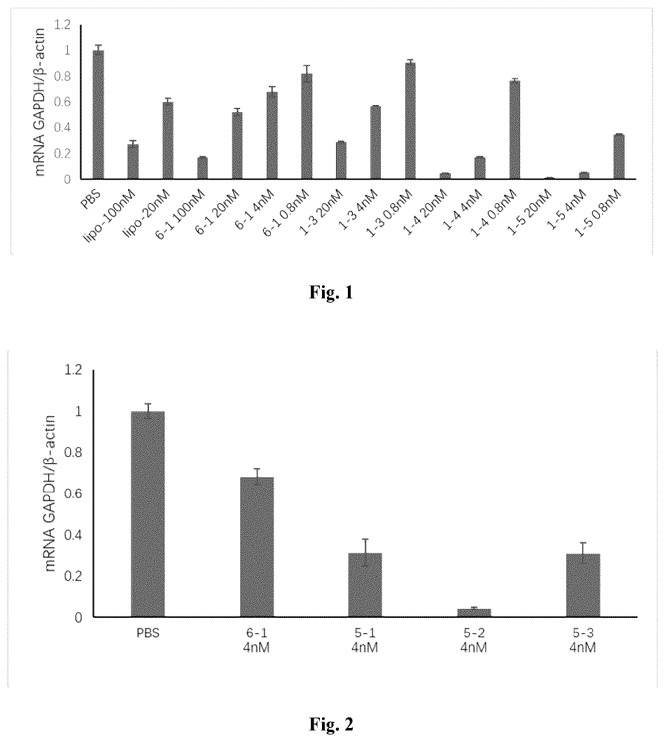

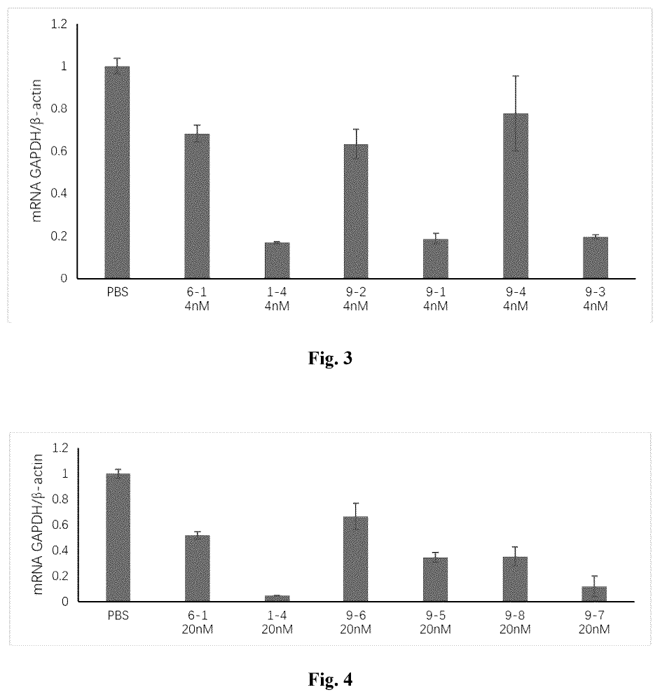

The samples were added to a 96-well plate or 8 consecutive tubes corresponding to RT-qPCR and detected by real-time fluorescence quantitative PCR instrument according to the instructions of the kit. The relative expression of GAPDH mRNA was calculated by ΔΔCt method with β-actin as a reference gene. The comparison results were shown in FIGS. 1-4.

### Experimental Example 2. Exploration on the Mechanism of CaLNPs Enhancing Gene Expression Compared with LNPs

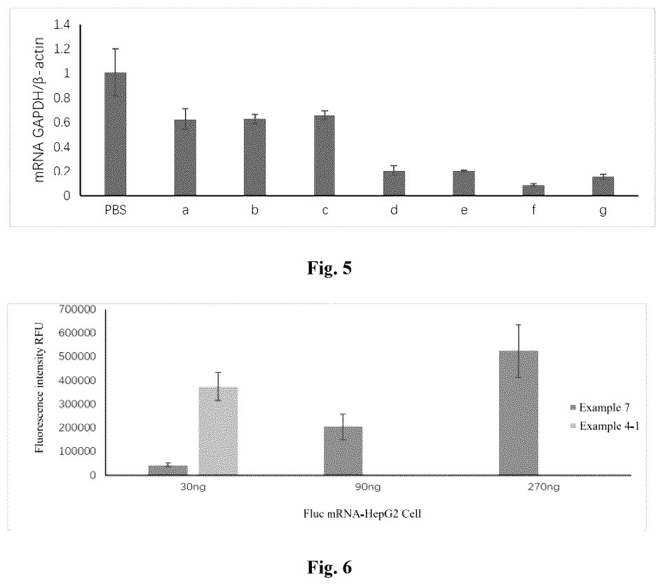

Cells were transfected with CaLNPs and LNPs with GAPDH siRNA as in the above experimental example. However, calcium acetate or calcium acetate encapsulated by liposome (A) was added to the LNP group (Example 6-1) to see if it could improve the gene knock-down efficiency at the cellular level. The results were shown in FIG. 5. It could be seen that adding the maximum amount of free calcium acetate corresponding to Example 5-2 to the control LNPs of Example 6-1 could not improve the transfection efficiency of the LNPs; whereas, adding calcium acetate encapsulated by liposome was helpful, but the effect was not as good as that of the CaLNPs generated after mixing in Examples 1-4. It was speculated that the lipid nanoparticles with gene encapsulated therein in the same endosome as calcium contributed to endosomal escape.

The agent preparation was as follows:

| | | Detection content * | | | | |
|---|---|---|---|---|---|---|
| | | encapsulation | Sampling | | 400 mM | |
| Experimental | Example | efficiency | amount of stock | DMEM | calcium acetate | Liposome |
| example No. | No. | (mg/ml) | solution (μl) | (μl) | solution (μl) | A (μl) |
| a | 6-1 | 0.0560 | 8 | 392 | — | — |
| b | 6-1 | 0.0560 | 8 | 376 | 16 | — |
| c | 6-1 | 0.0560 | 8 | 384 | 8 | — |
| d | 6-1 | 0.0560 | 8 | 277 | — | 115 |
| e | 6-1 | 0.0560 | 8 | 335 | — | 57.5 |
| f | 5-2 | 0.0033 | 129 | 271 | — | — |
| g | 1-4 | 0.0274 | 16 | 384 | — | — |

100 μl was dosed to each 6-well plate.

### Liposome A was Prepared as Follows:

30.0 mg of cholesterol and 30 mg of EPC were weighed and dissolved by adding 10 mg of ethanol (10 ml) to form intermediate A, a 400 mmol/l calcium acetate solution was intermediate B, A at 4 ml/min and B at 18 ml/min were mixed in a microfluidic chip, an intermediate was collected, and after dialysis by an 8 kDa dialysis membrane, an internal phase was collected, filtered, and sterilized.

### Experimental Example 5. Experiment of Cell Transfection with Firefly Luciferase mRNA

### Cell Culturing:

HepG2, a human hepatocarcinoma cell line, was left to stand and cultured in 90% DMEM medium+10% fetal bovine serum culture+1% Penicillin-Streptomycin medium at 5% CO₂ at 37° C. and observed under an inverted microscope, and the cells in the logarithmic growth phase were selected, digested with 0.25% trypsin, counted, and plated. (1) The medium was discarded, and after washing once with 2 ml of a PBS solution, the PBS was discarded. (2) 1 ml of 0.25% trypsin was added, spread evenly up and down and left and right, digested at 37° C. for 10 min, and observed at any time, and when the cells flowed down in a muddy state, the digestion was complete. (3) 1 ml of a medium was added, the digestion was terminated, the digested cells were repeatedly bubbled until they were detached and dispersed to form a cell suspension, which was centrifuged, and the supernatant was discarded. (4) The cells were re-suspended with the medium and counted on a counting plate. Each white opaque 96-well plate with 0.1 ml of medium was inoculated with 10,000 cells per well, and dosage was started after 24 hours of culturing.

### Dosage and Detection:

When the cells had grown to 70-80%, dosage was carried out. Positive control (mRNA): 4 μl of 1 mg/ml mRNA+6 μl of lipofectamin 3000 solution was made up to 150 μl by adding a serum-free DMEM culture solution; in addition, after the addition was completed, the materials were mixed until uniform, and the mixture was incubated at room temperature for 10-15 min and diluted to different concentrations in series; each agent was separately prepared with serum-free DMEM to the dosage concentration; and after the agent was prepared, 10 μl was added to the corresponding 96-well plate. Culturing was continued for 24 hours. The cell culture plate was equilibrated at room temperature for 10 min, 100 μl of Bright-Glo™ was added to each well, and the plate was incubated at room temperature for 5 min and detected for luminescence by a multi-functional fluorescence microplate reader with the detection wavelength being 562 nm.

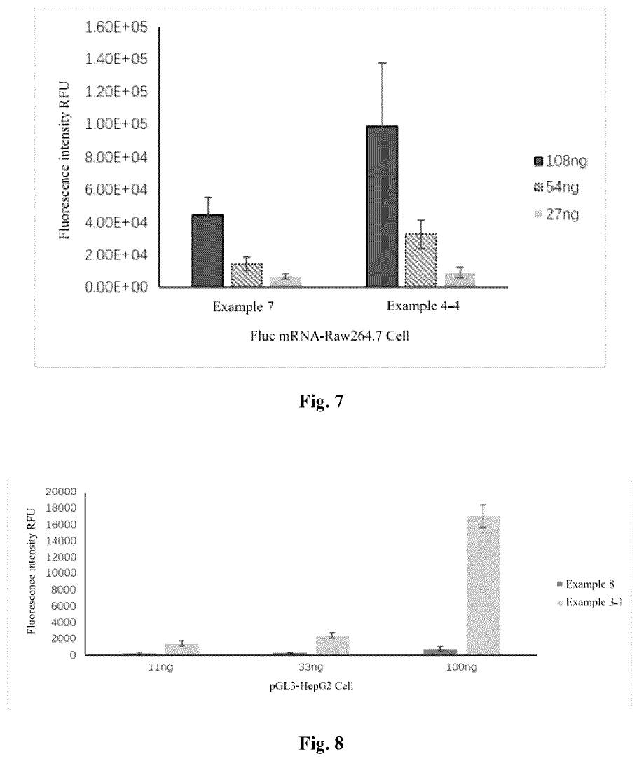

The results were shown in FIGS. 6 and 7.

### Experimental Example 6. Experiment of Cell Transfection with Luciferase Plasmid pGL3-Control

### Cell Culturing:

HepG2, a human hepatocarcinoma cell line, was left to stand and cultured in 90% DMEM medium+10% fetal bovine serum culture+1% Penicillin-Streptomycin medium at 5% CO₂ at 37° C. and observed under an inverted microscope, and the cells in the logarithmic growth phase were selected, digested with 0.25% trypsin, counted, and plated. (1) The medium was discarded, and after washing once with 2 ml of a PBS solution, the PBS was discarded. (2) 1 ml of 0.25% trypsin was added, spread evenly up and down and left and right, digested at 37° C. for 10 min, and observed at any time, and when the cells flowed down in a muddy state, the digestion was complete. (3) 1 ml of a medium was added, the digestion was terminated, the digested cells were repeatedly bubbled until they were detached and dispersed to form a cell suspension, which was centrifuged, and the supernatant was discarded. (4) The cells were re-suspended with the medium and counted on a counting plate. Each white opaque 96-well plate with 0.1 ml of the medium was inoculated at 10,000 cells per well, and after culturing for 24 hours until the cells had grown to 70-80%, dosage was started.

### Dosage and Detection:

When the cells had grown to 70-80%, dosage was carried out. Positive control (pGL3): 75 μl of a serum-free DMEM culture solution was added to 1.5 μl of 1 mg/ml pGL3+3 μl of P3000 solution, after which these materials were mixed until uniform to form A; 2.3 μl of lipofectamin 3000±75 μl of the serum-free DMEM culture solution was mixed until uniform to form B; and A was mixed with B, incubated at room temperature for 10-15 min, and diluted into different concentrations in series; each agent was separately prepared with serum-free DMEM to the dosage concentration; and after the agent was prepared, 10 μl was added to the corresponding 96-well plate. Culturing was continued for 24 hours. The cell culture plate was equilibrated at room temperature for 10 min, 100 μl of Bright-Glo™ was added to each well, and the plate was incubated at room temperature for 5 min and detected for luminescence by a multi-functional fluorescence microplate reader with the detection wavelength being 562 nm. The results were shown in FIG. 8.

### Experimental Example 7. Efficiency of Transfection with Luciferase mRNA and Plasmid DNA In Vivo-Living Imaging Experiment

C57BL/6 mice, aged 8-10 weeks, male, 25-30 g were selected. 100 μl was administered via the tail vein, and in vivo living imaging was carried out 24 hours later.

Drug concentration:

| | Animal | Example | Drug encapsulation | |
|---|---|---|---|---|
| | No. | No. | content, mg/ml | Preparation method |
| DNA | 1 | 8-1 | 0.0520 | 0.2 ml was taken, diluted by |
| | | | | adding 0.125 ml of PBS, mixed |
| | 2 | | | upside down, and administered at |
| | | | | 0.1 ml/animal. |
| | 3 | 3-1 | 0.0320 | Direct administration with 0.1 |
| | 4 | | | ml/animal |
| mRNA | 5 | 7-1 | 0.0687 | Direct administration with 0.1 |
| | | | | ml/animal |
| | 6 | 4-2 | 0.0160 | Direct administration with 0.1 |
| | | | | ml/animal |
| | 7 | 7-1 | 0.0687 | 0.1 ml was taken, diluted by |
| | | | | adding 0.2 ml of PBS, and |
| | | | | administered at 0.1 ml/animal. |

Living imaging operation: 24 hours after administration to mice, (1) 75 mg of potassium fluorescein was weighed into a 15 ml centrifuge tube, and 5 ml of D-PBS was added for full dissolution, with the concentration being 15 mg/ml; (2) the solution was filtered through a 0.22 μm sterile filter into another 15 ml centrifuge tube for later use (prepared immediately before use); (3) the anesthetized and depilated mice were intraperitoneally injected with a concentration of 10 μl/g body weight; (4) 10-20 min after injection into the body, imaging analysis was performed using an appropriate instrument; and (5) the mice were re-injected with a fluorescein potassium solution and dissected 4 minutes later, and all organs (heart, liver, spleen, lung, and kidney) were taken for imaging detection.

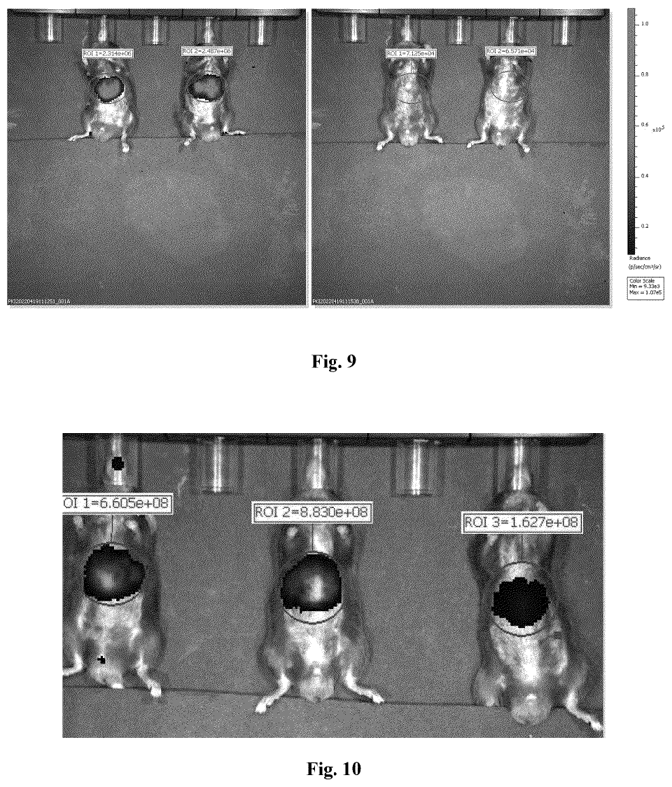

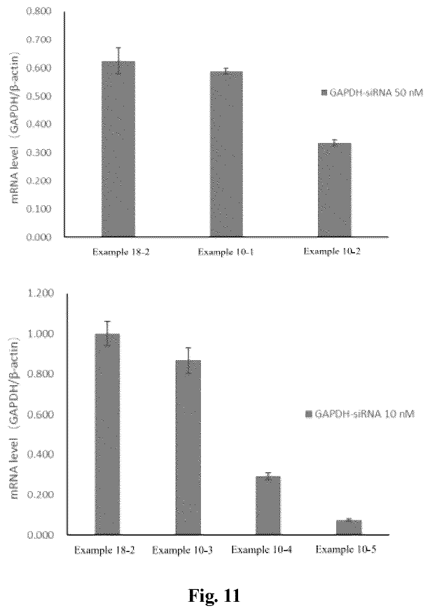

The results were shown in FIGS. 9 and 10.

### Experimental Example 8. GAPDH siRNA-Based Silencing Efficiency of siRNA-CaLNPs and siRNA-LNPs In Vitro

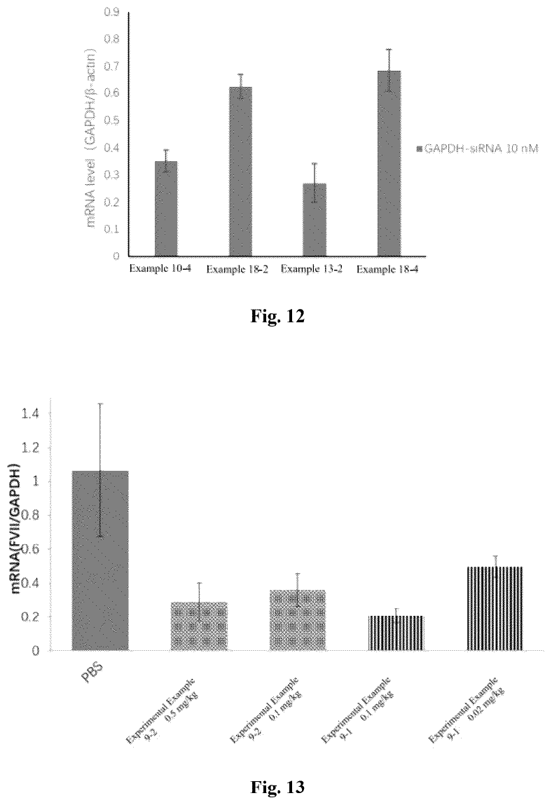

HepG2 cells in the logarithmic growth phase were inoculated into a 24-well plate at 1×10⁵/0.5 mL/well, and incubated at 37° C. and 5% CO₂ for 24 h for cell adherence. After incubation, the preparation sample was added to each well such that the final concentration of GAPDH siRNA in each well was about 10 nM. Cells treated with PBS were used as a negative control group. After further culturing in a cell incubator at 5% CO₂ and 37° C. for 24 h, the medium was discarded, and the cells were rinsed with PBS once to extract the total RNA from the cells. The inhibitory efficiency of GAPDH-siRNA on GAPDH mRNA expression in HepG2 cells was detected by fluorescence quantitative PCR, in which β-actin gene was used as an internal reference gene. The results were shown in FIGS. 11 and 12.

### Experimental Example 9. FVII-siRNA- and TTR-siRNA-Based In Vivo Silencing Activity of siRNA-CaLNPs and siRNA-LNPs

Preparation of siRNA-CaLNPs

32.5 mg of DLin-MC3-DMA, 31.0 mg of cholesterol, 16.5 mg of DSPC, and 8.0 mg of PEG-DMG were weighed and dissolved by adding 10 ml of ethanol to form an organic phase; 1.0 mg of siRNA (mouse FVII gene or mouse TTR gene) was taken separately and diluted with 9 ml of a 200 mmol/l calcium acetate solution (pH 5.0) to form an aqueous phase, wherein the pH value of the calcium acetate was adjusted to 5 with acetic acid; and 9 ml of the aqueous phase and 1 ml of the organic phase were taken and mixed into a tee pipe at a total flow rate of 22 ml/min to obtain intermediate I, which was dialyzed in a Tris-containing buffer with pH 7.2 by a dialysis bag with a molecular weight cut-off of 14000D. The internal phase dialysate was collected, filtered, and sterilized to finally obtain siRNA-CaLNPs.

Preparation of siRNA-LNPs

65 mg of DLin-MC3-DMA, 31.0 mg of cholesterol, 16.5 mg of DSPC, and 8.0 mg of PEG-DMG were weighed and dissolved by adding 10 ml of ethanol to form an organic phase; 1.0 mg of siRNA (mouse FVII gene or mouse TTR gene) was taken separately and diluted with 1.8 ml of a 25 mmol/L acetic acid-sodium acetate solution (pH 4.0) to form an aqueous phase, wherein the pH value of the 25 mmol/L acetic acid-sodium acetate solution was adjusted to 4.0 with acetic acid; and 9 ml of the aqueous phase and 1 ml of the organic phase were taken and mixed into a tee pipe at a total flow rate of 22 ml/min to obtain intermediate I, which was dialyzed in a Tris-containing buffer with pH 7.2 by a dialysis bag with a molecular weight cut-off of 14000D. The internal phase dialysate was collected, filtered, and sterilized to finally obtain siRNA-LNP.

FVII-siRNA and TTR-siRNA sequences

| | | |
|---|---|---|
| si-FVII | Sense | GAAUGAGCAGCUGAUCUGUGCAAAU |
| | Antisense | AUUUGCACAGAUCAGCUGCUCAUUC |
| si-TTR | Sense | 5′-AaCaGuGuUcUuGcUcUaUaA-3′ |
| | Antisense | 5′-uUaUaGaGcAaGaAcAcUgUu*U*u-3′ |

Lowercase letters 2′-O-methyl modification
Uppercase letters 2′-Deoxy-2′-fluoro modification
*Thiophosphate bond

Properties of Formulations in this Study

| | | | | | | | Encapsula- | ΔCa²⁺ | |
|---|---|---|---|---|---|---|---|---|---|
| | | Formula- | Nucleic | | | | tion | concen- | |
| | Composition of formulation | tion | acid | Particle | | Content | effi- | tration, | Chol |
| | Lipid name with mole % | type | type | size | PDI | μg/ml | ciency | μM | μM |
| Experimental | Dlin-MC3-DMA:Cholesterol:DSPC:PEG(2000)- | CaLNP | FVII- | 109 | 0.031 | 55.10 | 95.1% | 137.6 | 611.2 |
| example 9-1 | DMG = 25:38.5:10:1.5 | | siRNA | | | | | | |
| Experimental | Dlin-MC3-DMA:Cholesterol:DSPC:PEG(2000)- | LNP | FVII- | 77 | 0.027 | 91.29 | 96.6% | / | / |
| example 9-2 | DMG = 50:38:5:10:1.5 | | siRNA | | | | | | |
| Experimental | Dlin-MC3-DMA:Cholesterol:DSPC:PEG(2000)- | CaLNP | TTR- | 100 | 0.095 | 45.81 | 96.9% | 121.3 | 587.9 |
| example 9-3 | DMG = 25:38.5:10:1.5 | | siRNA | | | | | | |
| Experimental | Dlin-MC3-DMA:Cholesterol:DSPC:PEG(2000)- | LNP | TTR- | 68 | 0.08 | 74.12 | 95.2% | / | / |
| example 9-4 | DMG = 50:38:5:10:1.5 | | siRNA | | | | | | |

C57BL/6N male mice, aged 6-8 weeks and weighing 18-20 g, were provided by Beijing Vital River Laboratory Animal Technology Co., Ltd.

(1) The C57BL/6N male mice were randomly divided into 5 groups, with 4 mice in each group. After a week of adaptive feeding, administration was started, and the following 5 groups were set up: (1) PBS; (2) FVII siRNA-CaLNP 0.5 mg/kg (the nucleic acid concentration here); (3) FVII siRNA-CaLNP 0.1 mg/kg (the nucleic acid concentration here); (4) FVII siRNA-LNP 0.1 mg/kg (the nucleic acid concentration here); and (5) FVII siRNA-LNP 0.02 mg/kg (the nucleic acid concentration here). Each group was administered 0.1 mL/mouse by tail vein injection. 48 h after administration, the mice were sacrificed by cervical dislocation, and livers were taken.

(2) The C57BL/6N male mice were randomly divided into 6 groups, with 4 mice in each group. After a week of adaptive feeding, administration was started, and the following 6 groups were set up: (1) PBS; (2) TTR siRNA-CaLNP 0.2 mg/kg (the nucleic acid concentration here); (3) TTR siRNA-CaLNP 0.05 mg/kg (the nucleic acid concentration here); (4) TTR siRNA-CALNP 0.0125 mg/kg (the nucleic acid concentration here); (5) TTR siRNA-LNP 0.2 mg/kg (the nucleic acid concentration here); and (6) TTR siRNA-LNP 0.05 mg/kg (the nucleic acid concentration here). Each group was administered 0.1 mL/mouse by tail vein injection. 48 h after administration, the mice were sacrificed by cervical dislocation, and livers were taken.

The livers from each group were ground into a liver homogenate. 20-30 mg of the liver homogenate was taken from each group, and the total RNA was extracted therefrom. The inhibitory efficiency of FVII-siRNA and TTR-siRNA on the expression of FVII and TTR mRNA in mice was then detected by fluorescence quantitative PCR, in which GAPDH gene was used as an internal reference gene.

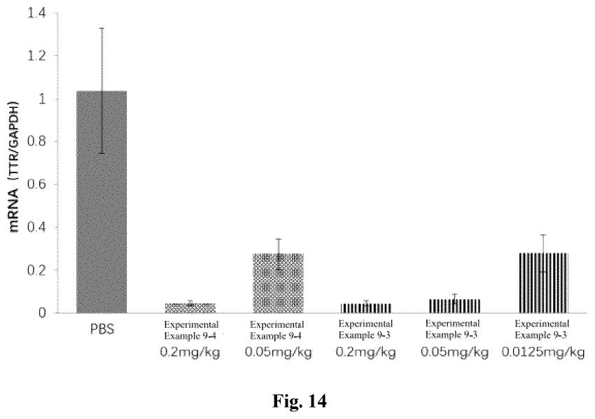

The results were shown in FIGS. 13 and 14.

1. A nucleic acid-loaded calcium-containing cationic lipid nanoparticle, wherein
the cationic lipid nanoparticle comprises cationic lipid, neutral lipid, PEGylated lipid, and cholesterol and/or cholesterol ester;
the cationic lipid nanoparticle contains calcium ions, and anions corresponding to the calcium ions are not phosphate radicals, hydrogen phosphate radicals, and dihydrogen phosphate radicals; and
the cationic lipid nanoparticle is free of non-PEG-group-modified negatively charged lipid.

2. The calcium-containing cationic lipid nanoparticle according to claim 1, wherein the cationic lipid nanoparticle contains calcium ions in a non-precipitated state.

3. The calcium-containing cationic lipid nanoparticle according to claim 1, wherein the concentration of calcium in the whole formulation is 0.01-150 mmol/L; preferably is, 0.01-0.1 mmol/L or 0.1-150 mmol/L, 0.01-0.1 mmol/L, 0.1-1 mmol/L, 1-10 mmol/L, 10-100 mmol/L, 100-150 mmol/L, 0.01-0.1 mmol/L, 0.1-1 mmol/L, 1-10 mmol/L, 10-30 mmol/L, 30-50 mmol/L, 50-70 mmol/L, 70-90 mmol/L, 90-110 mmol/L, 110-130 mmol/L, 130-150 mmol/L,
0.01-1 mmol/L, 0.02-0.8 mmol/L, 0.03-0.5 mmol/L or 0.1-0.5 mmol/L.

4. The calcium-containing cationic lipid nanoparticle according to claim 1, wherein the concentration of calcium in the cationic lipid nanoparticle relative to a total volume is 10-300 μM, 15-250 μM, 20-180 μM, 20-180 μM or 60-150 μM.

5. The calcium-containing cationic lipid nanoparticle according to claim 1, wherein the molar ratio of calcium to lipid in the cationic lipid nanoparticle is 1:(0.01-20), 1:(0.1-10), 1:(1-10), 1:(0.1-1), 1:(2-18), 1:(5-15) or 1:(7-13).

6. The calcium-containing cationic lipid nanoparticle according to claim 1, wherein the molar ratio of the calcium in the cationic lipid nanoparticle to the total amount of the cholesterol and cholesterol ester in the formulation is (0.01:1)-(0.8:1), (0.02:1)-(0.6:1), (0.03:1)-(0.4:1) or (0.08:1)-(0.3:1).

7. The calcium-containing cationic lipid nanoparticle according to claim 1, wherein the calcium ions are derived from calcium salts; the calcium ions are derived from a calcium salt solution with a concentration of 50-1000 mmol/L, 50-150 mmol/L, 150-300 mmol/L, 300-500 mmol/L or 500-800 mmol/L.

8. The calcium-containing cationic lipid nanoparticle according to claim 1, wherein the substance to be delivered by the calcium-containing cationic lipid nanoparticle is plasmid DNA, single-stranded DNA, double-stranded DNA, siRNA, shRNA, aiRNA, miRNA, mRNA, circular RNA, tRNA, rRNA, vRNA, gRNA, aptamer, ribozyme, oligonucleotide, or any combination thereof.

9. The calcium-containing cationic lipid nanoparticle according to any one of the claim 1, wherein the molar number of phosphate radicals in the nucleic acid:the molar number of positive charges in the cationic lipid is 1:(0.5-20), 1:(1-10), 1:(1.5-6), 1:(1.5-3) or 1:(3-6).

10. The calcium-containing cationic lipid nanoparticle according to claim 1, wherein the mass ratio of the nucleic acid to the lipid is 1:(1-100), 1:(5-90), 1:(10-70), further preferably is or 1:(10-30).

11. The calcium-containing cationic lipid nanoparticle according to claim 1, wherein the length of the substance to be delivered by the calcium-containing cationic lipid nanoparticle is about 15-30000, 15-60, 60-120, 120-250, 250-500, 500-1000, 1000-2000, 2000-4000, 4000-8000, 8000-15000, 15000-20000, 20000-25000, 25000-30000, 15-60, 15-50, 15-40, 15-30, 15-25, 19-25, 20-30, 20-50, 20-80, 30-50, 30-80, 30-120, 50-100, 50-150, 50-250, 100-200, 100-300, 100-500, 200-500, 200-1000, 300-800, 300-1500, 1000-3000, 1000-5000, 1000-8000, 5000-10000, 5000-15000, 5000-20000, 10000-25000, or 10000-30000 bases or base pairs.

12. The calcium-containing cationic lipid nanoparticle according to claim 1, wherein the amount of the loaded nucleic acid is 5 μg/ml-10 mg/ml, 5-10 μg/ml, 10-20 μg/ml, 20-40 μg/ml, 40-80 μg/ml, 80-150 μg/ml, 150-300 μg/ml, 300-400 μg/ml, 400-800 μg/ml, 800 μg/ml-1 mg/ml, 1-1.5 mg/ml, 1.5-2 mg/ml, 2-4 mg/ml, 4-6 mg/ml, 6-8 mg/ml, 8-10 mg/ml, 50±50 μg/ml, 100±50 μg/ml, 200±50 μg/ml, 300±50 μg/ml, 400±50 μg/ml, 500±50 μg/ml, 600±50 μg/ml, 700±50 μg/ml, 800±50 μg/ml, 900±50 μg/ml, 1000±50 μg/ml, 1500±50 μg/ml, 2000±50 μg/ml, 2500±50 μg/ml, 3000±50 μg/ml, 4000±50 μg/ml, 5000±50 μg/ml, 6000±50 μg/ml, 7000±50 μg/ml, 8000±50 μg/ml, 9000±50 μg/ml, or 10000±50 μg/ml.

13. The calcium-containing cationic lipid nanoparticle according to claim 1, wherein the lipids constituting the cationic lipid nanoparticle comprise one of or a combination of cationic lipid, cholesterol and/or cholesterol ester, neutral lipid, and PEGylated lipid, the lipids constituting the cationic lipid nanoparticle comprise the following lipids:
(1) cationic lipid selected from ionizable cationic lipids;
(2) cholesterol lipid selected from cholesterol and/or cholesterol esters;
(3) neutral lipid selected from phospholipids, fatty acid glycerides, or glycolipids, or any combination thereof; and
optionally, (4) PEGylated lipid; or
(1) cationic lipid selected from ionizable cationic lipids;
(2) cholesterol lipid selected from cholesterol;
(3) neutral lipid selected from phospholipids; and
optionally, (4) PEGylated lipid.

14. The calcium-containing cationic lipid nanoparticle according to claim 1, wherein
the lipids constituting the cationic lipid nanoparticle comprise 1-90% by mole of cationic lipid and/or 1-90% by mole of cholesterol lipid, 10-60% by mole of cationic lipid and/or 25-75% by mole of cholesterol lipid, or 20-40% by mole of cationic lipid and/or 40-60% by mole of cholesterol lipid.

15. The calcium-containing cationic lipid nanoparticle according to claim 1, wherein
the lipids constituting the cationic lipid nanoparticle comprise a combination of the following components in % mole
(a)(1) cationic lipid: 1-90%,
(2) cholesterol lipid: 1-90%,
(3) neutral lipid: 1-90%, and
(4) PEGylated lipid: 0.1-20%;
(b)(1) cationic lipid: 10-60%,
(2) cholesterol lipid: 25-75%,
(3) neutral lipid: 1-30%, and
(4) PEGylated lipid: 0.5-10%;
or
(c)(1) cationic lipid: 20-40%,
(2) cholesterol lipid: 40-60%,
(3) neutral lipid: 5-25%, and
(4) PEGylated lipid: 1-3%.

16. The calcium-containing cationic lipid nanoparticle according to claim 1, wherein the cationic lipid is selected from ionizable cationic lipids, the ionizable cationic lipid is selected from DSDMA, DLinDMA, DLenDMA, DODMA, A6, OF-02, A18-Iso5-2DC18, 98N₁₂-5, 9A1P9, C12-200, cKK-E12, 7C1, G0-C14, L319, 304O₁₃, OF-Deg-Lin, 306-O12B, 306Oᵢ₁₀, FTT5, SM102, ALC-0315, A9, Lipid 2,2(8,8)4CCH3, CL1, LP01, DLin-MC3-DMA, or analogs and combinations thereof; and/or
the neutral phospholipid is selected from one or more of egg yolk lecithin, soybean lecithin, hydrogenated soybean lecithin, phosphatidylcholine, phosphatidylethanolamine, phosphatidylinositol, distearoylphosphatidylcholine, dimyristoylphosphatidylcholine, dipalmitoylphosphatidylcholine, dioleoylphosphatidylcholine, distearoylphosphatidylethanolamine, dimyristoylphosphatidylethanolamine, dipalmitoylphosphatidylethanolamine, dioleoylphosphatidylethanolamine, distearoylphosphatidylinositol, dimyristoylphosphatidylinositol, dipalmitoylphosphatidylinositol, dioleoylphosphatidylinositol, 9A1P9, and 10A1P10;
the PEGylated lipid is selected from methoxy polyethylene glycol-distearoylphosphatidylethanolamine (mPEG-DSPE), methoxy polyethylene glycol-dioleoylphosphatidylethanolamine (mPEG-DOPE), methoxy polyethylene glycol-dipalmitoylphosphatidylethanolamine (mPEG-DPPE), polyethylene glycol-dilauroylglycerol (PEG-DAG), polyethylene glycol-dimyristoylglycerol (PEG-DMG), polyethylene glycol-dipalmitoylglycerol (PEG-DPG), polyethylene glycol-distearoylglycerol (PEG-DSG), polyethylene glycol-dioleoylglycerol (PEG-DOG), polyethylene glycol-dilinoleylglycerol (PEG-DLinG), polyethylene glycol-dilauroylpropylamine (PEG-DAA), polyethylene glycol-dimyristoylpropylamine (PEG-DMA), polyethylene glycol-dipalmitoylpropylamine (PEG-DPA), polyethylene glycol-dioleoylpropylamine (PEG-DOA), polyethylene glycol-dilinoleylpropylamine (PEG-DLinA), polyethylene glycol-ceramide (PEG-ceramide), stearoyl polyethylene glycol ester, vitamin E polyethylene glycol succinate (TPGS), and any combination thereof;
wherein the PEG is a PEG group with a degree of polymerization selected from 3-100, 3-50, 50-100, 3-10, 10-20, 20-30, 30-40, 40-50, 50-60, 60-70, 70-80, 80-90, or 90-100.

17. The calcium-containing cationic lipid nanoparticle according to claim 1, wherein the particle size of the calcium-containing cationic lipid nanoparticle is 25-1000 nm, 25-500 nm, 500-1000 nm, 25-75 nm, 75-125 nm, 125-175 nm, 175-225 nm, 225-275 nm, 275-350 nm, 350-500 nm, 500-800 nm, or 800-1000 nm.

18. The calcium-containing cationic lipid nanoparticle according to claim 1, wherein the calcium-containing cationic lipid nanoparticle is selected from the following nanoparticle formulations:
siRNA-loaded calcium-containing cationic lipid nanoparticle, with cationic lipid (preferably DLin-MC3-DMA), cholesterol, neutral phospholipid (preferably DSPC), and PEGylated lipid (preferably PEG2000-DMG) as lipids and calcium acetate as calcium ion source;
mRNA-loaded calcium-containing cationic lipid nanoparticle, with cationic lipid (preferably DLin-MC3-DMA), cholesterol, neutral phospholipid (preferably DSPC), and PEGylated lipid (preferably PEG2000-DMG) as lipids and calcium acetate as calcium ion source; and
DNA-loaded calcium-containing cationic lipid nanoparticle, with cationic lipid (preferably DLin-MC3-DMA), cholesterol, neutral phospholipid (preferably DSPC), and PEGylated lipid (preferably PEG2000-DMG) as lipids and calcium acetate as calcium ion source.

19. The calcium-containing cationic lipid nanoparticle according to claim 1, wherein the calcium-containing cationic lipid nanoparticle has targeting effects on liver, lung, or spleen.

20. A calcium-containing cationic lipid nanoparticle composition, wherein the calcium-containing cationic lipid nanoparticle composition is prepared by mixing calcium-containing cationic lipid nanoparticles with nucleic acid-loaded cationic lipid nanoparticles, and
optionally after mixing, adjusting the pH value to neutrality.

21. Use of the nucleic acid-loaded calcium-containing cationic lipid nanoparticle according to any one of claims 1-19 or the calcium-containing cationic lipid nanoparticle composition according to claim 20 for gene transfection into cultured cells in vitro-, cell modification in vitro, local injection into the body to achieve gene transfection, local or systemic injection into the body to achieve vaccine immunization.

22-26. (canceled)
